SEQUENCE LISTING (47 sequences)

SEQ ID NO: 1 - DNA, 1932 bp, Homo sapiens
  CDS (1)..(1932)
atggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgttcctcctggttttctcctgcctcgtgctgtctgtgttttccaccatcaaggagtatgagaagagctcggagggggccctctacatcctggaaatcgtgactatcgtggtgtttggcgtggagtacttcgtgcggatctgggccgcaggctgctgctgccggtaccgtggctggagggggcggctcaagtttgcccggaaaccgttctgtgtgattgacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattggctacggggacaagtacccccagacctggaacggcaggctccttgcggcaaccttcaccctcatcggtgtctccttcttcgcgctgcctgcaggcatcttggggtctgggtttgccctgaaggttcaggagcagcacaggcagaagcactttgagaagaggcggaacccggcagcaggcctgatccagtcggcctggagattctacgccaccaacctctcgcgcacagacctgcactccacgtggcagtactacgagcgaacggtcaccgtgcccatgtacagttcgcaaactcaaacctacggggcctccagacttatccccccgctgaaccagctggagctgctgaggaacctcaagagtaaatctggactcgctttcaggaaggaccccccgccggagccgtctccaagtaaaggcagcccgtgcagagggcccctgtgtggatgctgccccggacgctctagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaagaagcaagcctccccggagaggacattgtggatgacaagagctgcccctgcgagtttgtgaccgaggacctgaccccgggcctcaaagtcagcatcagagccgtgtgtgtcatgcggttcctggtgtccaagcggaagttcaaggagagcctgcggccctacgacgtgatggacgtcatcgagcagtactcagccggccacctggacatgctgtcccgaattaagagcctgcagtccaggcaagagccccgcctgcctgtccagcaggggacaagaacggggtgggcttctgggacaaagcccactgtggcccatggtgggagtgcagggggtgtgtgggcggggcctcctccccacccacgtcggcctctgtcagcttctgttgtgtcttcacaaagtctgttttaa

SEQ ID NO: 2 - protein, 643 aa, Homo sapiens
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFYATNLSRTDLHSTWQYYERTVTVPMYSSQTQTYGASRLIPPLNQLELLRNLKSKSGLAFRKDPPPEPSPSKGSPCRGPLCGCCPGRSSQKVSLKDRVFSSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRQEPRLPVQQGTRTGWASGTKPTVAHGGSAGGVWAGPPPHPRRPLSASVVSSQSLF

SEQ ID NO: 3 - DNA, 1878 bp, Homo sapiens
  CDS (1)..(1878)
atggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgttcctcctggttttctcctgcctcgtgctgtctgtgttttccaccatcaaggagtatgagaagagctcggagggggccctctacatcctggaaatcgtgactatcgtggtgtttggcgtggagtacttcgtgcggatctgggccgcaggctgctgctgccggtaccgtggctggagggggcggctcaagtttgcccggaaaccgttctgtgtgattgacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattggctacggggacaagtacccccagacctggaacggcaggctccttgcggcaaccttcaccctcatcggtgtctccttcttcgcgctgcctgcaggcatcttggggtctgggtttgccctgaaggttcaggagcagcacaggcagaagcactttgagaagaggcggaacccggcagcaggcctgatccagtcggcctggagattctacgccaccaacctctcgcgcacagacctgcactccacgtggcagtactacgagcgaacggtcaccgtgcccatgtacagttcgcaaactcaaacctacggggcctccagacttatccccccgctgaaccagctggagctgctgaggaacctcaagagtaaatctggactcgctttcaggaaggaccccccgccggagccgtctccaagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaagaagcaagcctccccggagaggacattgtggatgacaagagctgcccctgcgagtttgtgaccgaggacctgaccccgggcctcaaagtcagcatcagagccgtgtgtgtcatgcggttcctggtgtccaagcggaagttcaaggagagcctgcggccctacgacgtgatggacgtcatcgagcagtactcagccggccacctggacatgctgtcccgaattaagagcctgcagtccaggcaagagccccgcctgcctgtccagcaggggacaagaacggggtgggcttctgggacaaagcccactgtggcccatggtgggagtgcagggggtgtgtgggcggggcctcctccccacccacgtcggcctctgtcagcttctgttgtgtcttcacaaagtctgttttaa

SEQ ID NO: 4 - protein, 625 aa, Homo sapiens
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFYATNLSRTDLHSTWQYYERTVTVPMYSSQTQTYGASRLIPPLNQLELLRNLKSKSGLAFRKDPPPEPSPSQKVSLKDRVFSSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRQEPRLPVQQGTRTGWASGTKPTVAHGGSAGGVWAGPPPHPRRPLSASVVSSQSLF

SEQ ID NO: 5 - DNA, 1848 bp, Homo sapiens
  CDS (1)..(1848)
atggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgttcctcctggttttctcctgcctcgtgctgtctgtgttttccaccatcaaggagtatgagaagagctcggagggggccctctacatcctggaaatcgtgactatcgtggtgtttggcgtggagtacttcgtgcggatctgggccgcaggctgctgctgccggtaccgtggctggagggggcggctcaagtttgcccggaaaccgttctgtgtgattgacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattggctacggggacaagtacccccagacctggaacggcaggctccttgcggcaaccttcaccctcatcggtgtctccttcttcgcgctgcctgcaggcatcttggggtctgggtttgccctgaaggttcaggagcagcacaggcagaagcactttgagaagaggcggaacccggcagcaggcctgatccagtcggcctggagattctacgccaccaacctctcgcgcacagacctgcactccacgtggcagtactacgagcgaacggtcaccgtgcccatgtacagacttatccccccgctgaaccagctggagctgctgaggaacctcaagagtaaatctggactcgctttcaggaaggaccccccgccggagccgtctccaagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaagaagcaagcctccccggagaggacattgtggatgacaagagctgcccctgcgagtttgtgaccgaggacctgaccccgggcctcaaagtcagcatcagagccgtgtgtgtcatgcggttcctggtgtccaagcggaagttcaaggagagcctgcggccctacgacgtgatggacgtcatcgagcagtactcagccggccacctggacatgctgtcccgaattaagagcctgcagtccaggcaagagccccgcctgcctgtccagcaggggacaagaacggggtgggcttctgggacaaagcccactgtggcccatggtgggagtgcagggggtgtgtgggcggggcctcctccccacccacgtcggcctctgtcagcttctgttgtgtcttcacaaagtctgttttaa

SEQ ID NO: 6 - protein, 615 aa, Homo sapiens
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFYATNLSRTDLHSTWQYYERTVTVPMYRLIPPLNQLELLRNLKSKSGLAFRKDPPPEPSPSQKVSLKDRVFSSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRQEPRLPVQQGTRTGWASGTKPTVAHGGSAGGVWAGPPPHPRRPLSASVVSSQSLF

SEQ ID NO: 7 - protein, 872 aa, Homo sapiens [sequence omitted]

SEQ ID NO: 8 - DNA, 27 bp, Artificial
  oligonucleotide
acctctgcggattgcatcggtgtgtgg

SEQ ID NO: 9 - DNA, 25 bp, Artificial
  oligonucleotide
ggatgacttgcatgaggctgggtgg

SEQ ID NO: 10 - DNA, 35 bp, Artificial
  oligonucleotide
agcgaattctcaatgggcgaggacacggacacgcg

SEQ ID NO: 11 - DNA, 34 bp, Artificial
  oligonucleotide
tccggatcctcctgtgtccacacactgccacctc

SEQ ID NO: 12 - DNA, 35 bp, Artificial
  oligonucleotide
aatattaaaacagactttgtgaagacacaacagaa

SEQ ID NO: 13 - DNA, 33 bp, Artificial
  oligonucleotide
atcagaattcacatggtgcagaagtcgcgcaac

SEQ ID NO: 14 - DNA, 42 bp, Artificial
  oligonucleotide
tgacagatcttaaaacagactttgtgaagacacaacagaagc

SEQ ID NO: 15 - DNA, 17 bp, Artificial
  oligonucleotide
gtgtggatgctgccccg

SEQ ID NO: 16 - DNA, 18 bp, Artificial
  oligonucleotide
tcccgcctcaaaacctcg

SEQ ID NO: 17 - DNA, 35 bp, Artificial
  oligonucleotide
actagaattcagccagaaggtcagtttgaaagatc

SEQ ID NO: 18 - DNA, 27 bp, Artificial
  oligonucleotide
atcaggatccgcgccgcctcacttcct

SEQ ID NO: 19 - DNA, 35 bp, Artificial; identical to SEQ ID NO: 17
  oligonucleotide

SEQ ID NO: 20 - DNA, 36 bp, Artificial
  oligonucleotide
actaggatccctactggactgcaggctcttaattcg

SEQ ID NO: 21 - DNA, 31 bp, Artificial
  oligonucleotide
aactagaattcgtggaccagatcgtggggcg

SEQ ID NO: 22 - DNA, 27 bp, Artificial
  oligonucleotide
atcaggatccgcgccgcctcacttcct

SEQ ID NO: 23 - DNA, 28 bp, Artificial
  oligonucleotide
aatcagaattccaagagccccgcctgcc

SEQ ID NO: 24 - DNA, 22 bp, Artificial
  oligonucleotide
gcacgatgcacagttgaagtga

SEQ ID NO: 25 - DNA, 29 bp, Artificial
  oligonucleotide
tactgaattcttcctggtgtccaagcgga

SEQ ID NO: 26 - DNA, 37 bp, Artificial
  oligonucleotide
acatggatcctcacctggactgcaggctcttaattcg

SEQ ID NO: 27 - DNA, 37 bp, Artificial
  oligonucleotide
acatgaattccagaaggtcagtttgaaagatcgtgtc

SEQ ID NO: 28 - DNA, 31 bp, Artificial
  oligonucleotide
tgatggatcctcaccgcatgacacacacggc

SEQ ID NO: 29 - DNA, 30 bp, Artificial
  oligonucleotide
cacggatccagcagccagaaggtcagtttg

SEQ ID NO: 30 - DNA, 31 bp, Artificial
  oligonucleotide
cacgaattctggacggaccaaactgcgtata

SEQ ID NO: 31 - DNA, 32 bp, Artificial
  oligonucleotide
agcggatccatgggcgaggacacggacacgcg

SEQ ID NO: 32 - DNA, 34 bp, Artificial
  oligonucleotide
tccgaattctcctgtgtccacacactgccacctc

SEQ ID NO: 33 - DNA, 25 bp, Artificial
  oligonucleotide
gagcctcgaggacagccccagcaag

SEQ ID NO: 34 - DNA, 35 bp, Artificial
  oligonucleotide
aagaattctgtaaaaggtcactgccaggagccccc

SEQ ID NO: 35 - DNA, 31 bp, Artificial
  oligonucleotide
cggaattcccatggtgcagaagtcgcgcaac

SEQ ID NO: 36 - DNA, 32 bp, Artificial
  oligonucleotide
ccagatcttgtaaaaggtcactgccaggagcc

SEQ ID NO: 37 - DNA, 151830 bp, Homo sapiens
  misc_feature (10)..(10): n = a or c or g or t
attgcatcgntgtgtggcggcggggcatgcccagagcaccgggcacggccttcaatgggcgaggacacggacacgcggaaaattaaccacagcttcctgcgggaccacagctatgtgactgaaggtaacgtacgtgttgtctgagacccctccggccggccgcggcgtggggatgccgtcgcaccgaatgccctccgaaggtttggaccgcgcgatgtgtgtcgtgtccccccgccccaccccaccccaccccatcccaccccaccccaccccatcccatcccaccctgcccggggcccaggagggagggagcccgagggtaccggcctccgctgcccagcgccggcacagggcagcgccctcctccgcgccgccctccgggaggcagctttcctctcccaagccaggtggcatcctgattcggccctgaccataattttttaaaaggccacggctgtggctaatctggtgaagaaatctcggggaaatttaatggtttaaatcctggatttgccatttcagccctgcccaaagcccgcagaattttctaggctgccctctccctggagaagaagagggacccggggggaaaaaaacataatccattgccagatcctcctgggaggcccgcctgcccgggcccctccctgtcctccagaggcagggtccctgagtgggagggagaaggcggctggtttggggctggcctttttattcctggtgagttatattgagacaggagcagctgggctaactgtcgggattttccaaaaaagtgggacatgccatcccaaacaggccctgtttaaaatcccctaagttggccctacaagcccaacccccacccccaccctacccccgaggctggtgggtcagcgcccctctcttacaggcctggaacttccgggggccccctggtctgcctcgctaggggaacagtggggacagcttccgtgcgcaggcagggcccgcggagtgaccccgatggaggatggggaccgggaggtctgggctcggggccgcctgtgctggagccctgcccgagtgcggggactgtcagccgctaacccacgggctggcggcccggccgcagaagtgtgcgcggattccccgggtgggtgccccagtgggaggggccctgggcacgggtcccgcggggcaggtgcgcgtgggtgcctgtgtcagggagaccgagtgggccaggcggcggagaccgggctcctgcggctgggtccgcctcctcgaagcctggctctgccggaaatgaggccgagcggagccggagccccgcggaggcccggggagcgcagcgcgagcgcgggcgcgggcgggctcggggcgccctggcccgggaggcagaggctgggcgtgggacctgggcggagggaggctgcgggggccgcgcccgctcccggcgggagaaccgctgagtcacgcacgcttcgccgccgggtgtgtgcgagagaggggcagggctgcccgggcctccgggccggtggggctctgacccggccgcggctttgggaggcccggggagctgagagcggtcctttgtcgcctgcttcggcgaggctgagtcgggatcagcgtgggtccgggatgtggtttctgctcgcagcctgcagcgacagagggttggaggaagccgccgggttgctggcccgtgcctcggtggcctggctcgggccgagagcggatcttggcaggttgccccgctgctcccaggctcgctgtggtctggcttcctggagcaagcctctgtttgctcatctctacgttggggatgacggtgggatggggcgcagggctcgttgtgggaccaggcgtaaaaagcgccatggtggacattttcaaacattaattccctctgcaccctgcccgtccccttttctttcataaattcaccagccctcagcagggcacaggaacggggacggcacagttctggaggtcagagcagtgctatccaatccttcatccagacacgagatatttaccgagcacctgctgcatgccgggcactgttttatccctggggactggacaaagactccctgcccttgtgcggcctgaattctatcagggagctatatgaccttgaggtctgtcttctcaccagggctcagtctccccatctgtaaaatgggagtgaatcctgcctacagggtctggaggtttctgtgaggagcaggtgcaacggtgttgaaggagccgttcagagctatgcattgctaacgtgcagccaaaggatgctgagcacctgctgtgtgctgggacctcatacgaaggctcacctacgccttatccccaccttgagatgaacaaaccaaggcctggccttgtgcctggtccagaccgagtgccagtgtgtgcagggcatagggctcaggtgctgcggatgcagagagcaggatcggaacaagaaacaataataatcattataataggtagaattctggcctccctaatgaaaaacacactttcacactgttgcatcctgactatatttcatgctcagagcacagggaaggtcacagatttgaatgtcagaatgtcagacttgaatcatgttaaagtcctgcctctgactcctgactgctgtgcagccttggacaagttactacaccttcctgagcttcactttccctttgtaaagggagaaataataacgacctttcatacagggttgctgggatgatcagtgattttgctaatatcaaaagtgcccagcacagtgcttgggttgttggaggcattgaacacacggcattgttattatttatatgccttgtaactggaagagcctgtgggcaaacagtggatgctaaaattcagtttgtggaagaaccaggtgcacaaactcctgttctacctgtggttgagtctacactcccccaccacaccccagctgctctgatctacctcctgttccttgagcaggccattttctttcttgcttcagggcatttgcaatggctgttccctggaatacccacctcctgcctttctcaccactgactctttctcatcctttgggccccatgtccaatgtcatctttgcccgtgggagccctgcttgggttcctgagtctcttgtaaaatctcaaacatcttaggaagagtttaggttttgttagtcattcacatcttggtgtgaattcgtcagagcaggttggattttttttttttttttttttttttttggtggcagggctgggtggcttactcttggctgggctcaactgcattgagggttatggtgatgattaggtgtacctgcaggccacctgggggcacagagaactttgcatgaatggggtcagggtgtggggagagcagtgtagctgatagggcactgggccctggaccctggaccctggagggtgctaggaagtctccatctagttagacatttctcaagagctggatatggttccaggaaggactcttcctggctctgttctttctctttttgcggcaggcctcagtacttagctttagagtgagttgaataagcacttccagactagactgaacttacaaaacccatccatccttcctttgcttagcaccatttgccaagcacatcctgccctgggcagcagtgatgaccaagacacaggagctcagccaaggggaggtagttcaggctgaccaggtagatcctggcagtgactgggtctttctggggagcaacagtcactctcatgtgcctgacaggtaaggcacttccggagcacacttttcagctgaggccagaggcacagcttccccttgtttgagcatctcagccatcacacactgttggacactcatgctttttgcatctgcataaaggatgtacccatgttttcctaactcccatccctggatgtcctgtgtgatttcagagtagtctgtttattcccacagagttgtcctgattttatagttgaggaagtgaagggtgcttgggaattgctaaggtcatcatggggtcctgaggctgtctgaacgcagcaggcaggcaggttttcctgcatggaattgtctgcaggttttgaactggttgtgccctggataccatctactttctgcccaggaaaccacactgaagaggggatgctgcttgtgggagactcagcattcagtgaaatctgcacccttatggtggacgttgcagctgatctccacaacagttagtctctcttcttccaaggagaacacatatggttcctagtaagagatctcaggccatagctggatgtgatcagtgcaaggatgatctcattccccttgcagtgactggttccagaatggatgtgggagtcagttctggaggttgagtggaaagtagggagaaccacaggtagttggtccctctcttgtgatgggaacagggaaaccttgtggtggctggcagccatctgacaccatgaggggagcaagtcttaggccaccaccgttgatggcagggcagagagtgagtgagatccaggacatagaggcctgatgtccaggccgcctctgaacttccagttacatgagctgttacttttccttttcgttgtgcactttcaagtctccgttacttgcagcccatagcaccccaactcgtacagccctgactgcttatcaagaaaaccaggaggctgatctgtaacccacagctaagactcgagatacataaaacccagagggatttttgtttgctttctgttttgagaaccctctttcatttcactcaagtcaaggttgccaatttctggttttccctcctcagtcctattgaggaggtatttttggtcagataagagagcctgagatctggcatcgggagatctgggttcctggtggggtctgaccctgggtctgtaatattgggcaggtcacacggtctctctgagcctcagtttattcttaatagatcagtaagtgatgatgtctattctttaggggccagagttagactagcccttagaactgtcatcagactggctagcctaacttccaccccaacctatggggaaactgaggctaagaggggggacattagtggtggcacagagggagattagggagagtcagcctttgacttttgaaaatcaagatgtggactgttcagattctgtgttcctttcgcttctgtttggaaacgccacttacataagcttcccttgggcttcacagcgagtgtgggctccatttcagtgctggggtgccctccatcaaatcacatcactttctgagcctcagtttccctgcactgcctggtgccctgggttgtcatgaggctgtaaggagccaagggcatgtggactctgaagttctacacgtgtaaaaggcgactgctgtcaatttccccaaattgtagtgtaggccanctcccacgcccctgctgtaagcaatgtgctcctttctttttaatcaacccgaacttaaagcttggcgcagtcacagagcacggtttttgtcattttctttctggaagatgaaaatcaaattctaataaaatttttccactccatctcctgactgttgtcatttgctacactttttagtgtttatccttaaagcttgcagagctaagggatgtttggtaagaagtgtttaggccttgagactggaagagccgctgtatgaagcgctaggctccatggagagtgtgatggggagtaagagaaggagagtgaactcctgtgagtccaccaagcagagggacatactctcagcaactggggtatttccttccagtctttttttaatgcccatgtctgtttttaatgaaaactgtaatctgtctgtatcaacaattttgaaggctacttttctagtttggcatgagattataggaattttccagggctttgctccagggctggcttcatggatatgcaacctgtgtggtcatctaggtccccacactcagaaggacctgtgcttgggttcatgttctgctgttactatcttgaaatttttaagaatttcactttggatcctgtgttttgtaagcgaaatctgatgggacgggggagcctgggaatgagcagaggggtacgtgcggcaggcgagtctgtggttacacacattggctcccacagcacgctaccctgtgttcacttgagcgtctgagccccacgcacagtggacagctcatgcaccttctcagtgcgtctgtagtttcacaggcaggggccatgctttcaactgatgctccaaggcattgatgttctcatgcagtgacttctaagaaacatgaatgacgcaggaaccctatggtgtcctttcttaacgtgtgttacgtgcccatatttgccaatcgctgacactgaaaatgattacacagaagggaagggaaaaagagggcacctatagttccttttcctcctagtccttccttgtttattattgaaccaagggtagagggtgttggaagaatgtacacatatccagaaatgaaataagaacagttaagttagtttcctctgctctggtaagaacaaaatccatatgcgaaatataacttgtgcattttggtgagtctacattccagttacgtgctcttatgtttgcatttaaaattggaggtgcacactagaatggtgagtgataaaatgcacgctgagagtttaagcttttttttttttaaatttgaattgacattaaatagcaaataacaccatgacaaaatatggaaggcatgaaaaggctttgtatcttagcacctttaatgatgcttttcctttgctttttgaataagaggctctgcattttcatttttccctgtgccccacgaattatagggttggccctgctttgctggaactctgggtagggtgaactccttggccctgccttctacctccttggctttcgtgattggtggaaatggtgagagcctgccatccattgggcagctcttccctatggggaggggttacaatattaaatgtccgtgctcctctctcacccattgggtccatgggcctctgacttccaggttgctctacaggggagaggccaacagttatcttattttacaagctagaagttagacctacctctccccgcaggaccaaggggtggggaggtgaagtaggggtccatagctcttttttttcctacaggtttaccattaataaagcagatttttttattcctgtcttgcggccagtcttttctgtgtaatcccaagtcccagcaaggaagaggggtgactggttaccttggccccgcccccaagtcacacagtacacgtattgacaaccatcatttcattggctgcatactattcctgtgatacatgtgtcatcattcagtagttcaattaatcatttccttatggctggatatttagttccctcccttccctccctccctcccttcctcccttccttccttctctctctctctgtctctcccccacacactttcttattttcataactaacactgcaggaatgaacaccatttgtgtgtgtgaagaccccctactcccatcgttcccccttaaccttggaatgatgctcttcagaaggaattccacacgtgctatttcatataaagatcactagcatttttaatgttgttgacacacattggcagattattctccagaaagtttttctctttaaccacatgctgatcaacattaggtagtctaatttgtttgacctttgcaaatatcaaagatgaaaaatatttgttgtaattcacgtctgcataccaatgaggttgaacattttttccttttgttcagcgggtattggctgatccctgttatgtgccagggtgacattagattctagacatacaaagttgaagagcaaggctgtgtctgccttgaaatgtggactctcctctcccagtgtcttgggtgccactgcgggacctagctacttctcaggagagagtctgaagctgctggaggagtctgtacatccctcagcaccctggggagtcctgttagttacacagggccttgcaaagacaggcatctcaccattaagcccttgagcagaatgatccaagtggactttaggatgaaataattacaaaacaaacacaatagccattattatgattcaccatttattggctntgtactataatactatatgctggacatgaggctgcgtgcatctcattggtgatgccccagacctgatgttgtgtatattatttccccattttacagaggagaaaactgaggctgaggtgcttgttcaatgtcacatggtttgtaagggacagagctgagttttgaatgcagcctgccaaacccacgctcctgactgctacattaattggctgggacccacagggcagggttaggtggcctgttcagttctgattatgcaccacacttaggaggttttgggatttgaactagactacctggcttggtgctcttgctatctgtgttgcgatgaaaggcaatcgagaggggtcagcaggaggagagacccccttcacggcacaggtagatggcagtggcagaggccacaagtggacacgtggtccagggactgaagggcaggtggcatttccaggttggggggtgatgatgtgctgggacatggtattctagcgcattctgggcaggaggaacggtgtgtgcaaagtggcataacacttgtcccctgacagtgtccggctcctctgcttgagaccaggaggcagttagttagttgccagccggcccgggagcaggacacacctgggggcccctgtgctgcccaggacaggcactgccctccttgcacagtgggggccattgtcctccagaacccagggctgaatgtcccattgaggcagaacaaaggctgctcagaggttcccagctggggtgtgggctgccccaagcagggatgggatctccaactgcaggccaagggcctcctccaggctggcctggcctcctgcagccccacccccacccctcctcaggaccaggactgttgctgctggaggctggacctgggcttgggctcccaggcctgtgctctgggtctggcacagctgccgtgcccttggcagcttcttcatcccctgggcaatttccccatctatgaagcagagagagagcgagctcccaaccagcaaggctttcaggcagaattgaatgaaatagtgcacactctgtaggttaatctcaataaaagggagctctttcatgatcatgattagcttctgaatgtttttttacaatttcaaaaaagttttgatgcaaactttcaaatttgtgccacttctgggccaaagtgtttaagaagggagtgcaccttcccccctccctctgtcccagagaagggagagatgccgctccctggagcccctcatcactctgtggaagggactgctggccaactgttacaagaggagaaacttctttgcatttgtgagaaaatagtctattgaactgcttccaatctatcaagatcttgctgtacttccttcatttactctcccctgcttttggctgaagaatttttaggcaaatccaagactcctgtcgtttccccgttccatctgcaggcatctctgagtgttgagggcatttcttgtagcccagtgctgttatcccacctcacaaaacatatcctgattctttgttacctaaattctggtccattttatgaacgtccccagttgtctgagaaatgtctcttatggttgggtggtttggcccaggatccaaagtcctgcacctgcgtttggctgttctgtctcttgtcttttctgacctagactcagcctccatccccttttcaggctaccaacttgttgaagacatttgttattattttattttattttttttaagacggagtttcactctgtcgcccaggctggagtgcaatggcgtgatcttggctcactgaaacccccgcctcccgggttcaagtgattctcctgcctcagcatcctgagcagctgggactacatacaggtgcccaccaccacgcccgactaatttttgtatttttagtagagacggggtttcaccatgttggtcaggctggtcttgaactcctgacctcagataatctgccttcattattattttagagattacagtgtcactcccactgtccctaatttgtgcctggattccatttgccctgtgggtctggaaggctgagaggtggttgctgggacctgggcatcggccttggggctgcccctctctcctccaggacccctttctgcagagtggtgccctcgccactccctggctgagtgatcttgggcaagttgtccagccaggccgtgcctgggtgaccacatctgagctgggggtgagcgtggctgcancatcctctctgggatgtggtgggtgttgaatgagatggtgcatgccacgtgctccgtgggcctggtgcctgtgggtccctgtcttacccccatgatggggatgtggcaggaactggggtagccaccgcctgcccacacagtgctcactttctgtaggggagacacccctcagctggtcactacatacagcaggaccagcactttctgagggaagagggatgttctcttgggaagtctggatgctgaagacagtttgttactctgattaataccagttacaaagaaatccccacattccaggggttgatgtcatagaagtttatccctttgtaacagttcattgtggatgatcccagttggcccaggagtctcttccacagagtgatgcggggctccagacccttctcatctgtcagctcccatgtcctctccattctccggggaagagggtatggggaaggtgcactccctccttaaacactttggcccagacatggcacttgtgacttccctcacattccattggccagagctagtcacatggccccacctaatgcaaggggctctaggaaatgtagtccctggctgggcagcccagtggctactctgcagtgggaagaacctgcatcttggtggatgtcttgccatctttgccacatgaccccacaaaacaaacctttacattctcagtccaaaaaaccctactaagaatcctgtgctggagacaccctcactcaacccctgaccctcccctctccctgcttcagtgtccacacgtgcacggtgctgtgagatgcagagtccagagtcatgcggtggctaggaggtcagggacgccttcctgggagaggcgatgtctgagctgagtctgcaaagccaaataggtggtgcccaggtggatcaggtaggagagggattccgggcttcggctgcagcaggggtaaaggctggtgtcttgggagagggcatcctgtgtagagaggggtctgtgggccactgagatttagaggatgtgtgtggggtggggtggagtgggagaggagctggagcgggatgggaagtgggaggcaggactgtttgtgaaaggcttcaaatgccgagataaggagtttggattgtatcctattgacattgtggaaccagatggagatggggcatttccctttgtttgaaagtattttgatttctattggctgtcttactacaaaaaacatatgtagtcatagcaaaaagttcagaaaatttagaaagagaaaaggaggaaaagaaaatcctaccactgaaaatattttggtatatgtgtttttgcctatgggtatacatactatctaggtatatatatattcctacatttttttattcactgaaagatggtttttgagcatctactgtgtgcgtatcctattttgtaatctttaaaattttctcttaatgatatgggagctttctagcttagaaaataacacagcccattttcttagcttgctgggacttctataacacagtgccacataccgggcagttgaaacaacagagacttaccgcctcacagtgctagagcttgggagtccaagatcaaggtgttggcagggttgattccttctgaggcccctctccttggcttatagatgaacatctccctactgtgtcttcacatggtctttcctgtgtgtgtctgtgtccttagagggacaccaggcatattgagttagggctcatccatatgactgcattttaccttcgtcaccttgttaaaggtcctttttgtcaaacacagtcacattttgaggttctaggggttaggacttcaacaaaggaatcttggggagggggcacatttcagcccaaagcacccgtcatcattggtaatatcagtcacatgcttcttctatgtattttaaagagttccatgctggtgggtatcagaagttatggttggtttaggatcatgagcagtgttgtgagggcatctttgcacctttttgcgtgcatatttggttatttccctagggtgtggaatggctgcttctgagagtttggtctttctgaagtatcttcatctaatgtcaacttttcctgatgaccaaacttggtactaacaactccctttccccagagcattgctagtggcgactagatccaacatatttaatatttgccaatattatggtaaaaattgctgtttttttttgtgtgtgtgacagaggcttgctctgttgctcaggctggagtgcagtgtcgtgatcttggctcactgcaacctctgcctcccaggctcaagcaattcttgtctctcagtctcccaagaagctggcattataggctgtgttcttttaaagcatttttcagtgagattgaacttcctttattatatttattgaccatggcatatattcaattgtaagtttttttgtgcaccgttcttggcctgtttctctactgtaattgtcatctctcttgcaaacactgacccttggtttgttgtttctggtagcattgattttatgtggttagatgtcatgactcttgtcctgctgagcatggatctcccattccacaatcatgagaagaaccaacaatatggaggaggtgacaccgtgtagctttggaggctaagtaaaaaataggtcatgtgcttctgccttggagccagccaccatattgtgaggaagcccaagcagtttgtggagaggcacatgcagagaggaaccaaggtcctggttgagctgcctgcctaatatccagcaccaccttgccagcaggtgagtgagccatcttacaggtagagcctccagccctcaggcaagccatccatatggaacagagatgagccattcccaccaagatctacccaaactgcagattcatgaaccaaataaatggtttctgcttgaagccattaagttttgaagtgctttgttacacagcagtgggtaactggaagagtcatggattcctgacattgaattcctggtcctcctccttctcaggctacttgtctagatgttctgttctctccatgattctgtggatccctcagagccttccggtaacttccttctttgcttgtgttagcctgggtcaatctctgttgcttataactgacagacatgggaaaccagccccagcaatgagaggtgacccagctcagatcatgagacaggacaggaatccaggcctttctgaaacatagcccagggtcccatcccacaacgtgtcagtagacaccatgcctgctgggtcatgcctgcttccgctgcaccctgcacccagctcagcacctgctatcttccaaaggccattgctgattgcttgtacacacctgttagttcatgcacagacagcaaagcacgtagttgtgctgcctccttgccttcctgctatgatctgaatgtttaagtaccccctccaaattcacaggttgaaatccagacccccaatgtgagggtatttaaaaggtggggactttgggagtgatgaggttgtgagggtggagccctcatgggtgggattagtgcccttataaaagaccttagagagctcccttgcccttctgtcacgtgaggacgcagcgagaaggcactgtttgtgagtcaggaagttggacctcagtggacaccaaatctgctgtgccttgatcttggacttccagcctccagaattgtgaggaataaatgcttgttgtttataagccacccggtctatgatattttgttatagcagcctgaacagactaagccactcccagtgatgagcctgcatgatgttttacacaaacagatcactgaaagaaggaattggccagcaaagatgatgctcagcagagatgtgaaagatgttaatgctggaagtgaaatttaaattggaggtaaatggagtcatagaagaaatccatgatcttgggaagctgaagctacccttcaagaagctcttatatgcagccagaggagttgagtgaaggtgaacacactgatgtaaaccaggaaaggagttgtgccccaaagcatggagatgtcccagaggaagcgaggctgggaaaaacgttaaaggaactcttgaagatatttcacagtgttgaaagtgcaaaggataaaatcttggaagctggtctggagaaagataattctgcaaagcatagaaagggtgcttttttggtatcgtaaggtatacaataacagtagcgagcactgtgcaaactctctccatatgtcttttacaaagaaataaagcanttgacatctcaatgtttctaatgctttaaattacattgtaccaaataaatattagttgtactattttaaaaaaactttcccggttgggcattgtggctcacacctgtaatccaagcactttgggaggctgaggtgggaggatcgcttgagcccaggagttcgagaccagcctgggcaatatagtgagaccctgtctcttcaaaaaataaaaaaaaattagccaaggatggtggcatgtacctgcagtcccagctactcaggaggctgaggctggaggatcacttgaacccaggaggttgaggctgcagtgagctatgattgcaccactgtactccagcctgggtgacagagccagaccctgtctcaaaaagaaaaaaaaattccctgtgcattccctatggacatttgtaactgtccataaaagactttttaatgtcttgacaaaaaattttaaaggccacagaagaattgtaatttcctcattgattattaggatggctttaaatggttttagctttcatgctctatttttttttttttttttttgagatggagtctcgctctgtcgcccaggtcggggtacagtggtgtcatgtcagctcactgcaacttctgtctcctggttcaagcaattctcctgcctcagcctcctgagtagctgggattacaggtgcctgccacggcatctggctaatttttgtatttttagtagagacggaatttcaaccatgttggtcaagctgatcttgaactcctgacctcaggtgatctgcccgccttagcctcccaaagtgctggaattataggngcaagccaccgcacctggctcatgctcatttttatggatccacaccacccgtacagcaaggactgcctgcactcattccaagtggtcagagtggtcaccgcatgggccctccacgtggccnggccacagtgatgtttcaaaccctggctgggggattgcattcaatatccccttattaaaggcggcagctcaagaatattaaatcatgggaattcctcactgtggaagtgggaaggcagcccgtggttcaccgtgaggggcacccagagctccccctccactgcgttcagtgtgcagccctccagccagcctgtctgcttcgggagcaatccatcatggaatgaactgaccgaaggagcgaggggctgaatgatgtggttcctacgccgactttcaatgtgaaaggtgataaaaacagccctgaatattttatggccccaaggagaggtaaggctctttattgaagctgtgaaaatataatccatcatgataatgtgtcccatatcgtcagactctgcgagctaagttgtgtgtatgttaaggtgctctttttgagaagatcttaattttatttctctattttatttcattacagaaacgtttgaacgtgtagacagtagaataaagggggggggggaaataacatccttcatttcactactccagaaacactgctcacattttgttgcatttccttctaactaccccctgcagattgtactatgttttgtgtcttttaaaatattaaatgcactgtcaacattttcccagatcatcctgcactctgagttaacaatttttattggctgcataatacttcacgatgtaggtattattattcattcaataactatttatggattcattgatccgattagtgttgattgtctataacaggtgtgtgggtggggtagggggattcaggaagaaggaatagggcgcagctcccccnccccaggatttctggaannagggagacntaacagatggctgtgatccggtgtgaggggcagggatgcagtgggcccggttgcagcctgggagcaggtggtcagagatgnaaggctgtgggcagcagtggttggtggtccacagcagtaggcagcagggggagggggcgacattcatggcaggagggacattatgggcacagcacggcgtggtgtgttcagggttggtggagcgttcacttacagtcttgcagatcctggcaggtgtggtggtgatctgtccaggcaggggagggagagggaactgagagtcatcaaaagtctctgggagtttggaaaggagagtagggggctcagagggagtgtgagcacttccagcagaggtgagaaagcccccagtcagttgcccagggtgggcagtggaagggaagtggaggtgaacgttgtggggtggagagggttttcaggcaggctgggagctgcccagtgtgctggaggaaggctgggtctccttgaatggtgtttggtcaatgcaagaccacaggagtgtgactagaaggctgggggtgcagatggtggcaggtggaggatggagagagctgctcccactgctgaacaacgactgccccaactttatgggatgagcattcttatgaatgcccattgtcctgtattccagattattcttgtgtctgtccaggtagggatgcaatttcttgatgtaaggctataggtgtttttaagggcttgcataaagatttgaatatgatgttgtctagtagagtaaaaatcaaattgggcaaaacatttttgtttgggtgatttttggaagagtaagtccacgaatgcaacgcagctctggagtcatctgtagattacagcaagcccatcagtctctatgtctcttgcttacaacaaaggattgatttcagctccagcactaggtgacttgtgctgtgttcattatctcttgataggtgtctgacaggagatggggcttgggctgtgccagggaggagccgtgtggtgcaccacctatctccgcaggcataactattttgtcttcatggcaaaataatagcgatgatggtgatgaggagggaagctaccatttcttgactgctcctgtgtaatgacatgttggtgatcacattagggctttatgtccactctgggaggtggtgagaatgacatcgcgtttgcacatcaggacgctgagcctcagagaggttgagtcccaggacgaaggccacacagtgagtgccacaggtaccattaccacctaacaaatgactctggagctcagtgatgtgatgagaaccattttattctgtctcctggattctgtgagtcaggaatttgggcagagcttggctgggcaaaccttctgctctaaatggccacccatgagtcttccaagctggtgggtggactgatctggagggactgagatggtctcactcacacattttctgcggggtggggagagttggaaggcggggctcccctccccagcgtgcagtctcagggcagttggactcctttgatggcagctggctttcccgaatcaagtatccccagatacccaggtggaagctgcttggcccttcctgacccctgggacatctcagtgtcaattgtgtcatcttcatttagttcaagcaagtcacaggccggcccaggttgaagggcagttggactcaacctttgcatgtgggaagggccaggttacatggtagaaaagcagatgggataggagaccgtgctctggccctccctgggaaacacggtgtgccacagacgccctgggcagagccaagacccccactgggctctgtctgaccttggagccactgccccgctcctgagcaacaccctcttgtccccctgaacagtcacaggaagaacgggtccctctctccatgccattttcctgttaaaaaatgcaaaaacatcccatacttttgctcatttaaacacagaggaaaggaggtgagtgaaagctttctttaggggtagattagatgtgaggcagaccggtggccctgggtgtgcaccgggtggaaattattcttacaaacagggccgggtgggggtgcagcctgccaccgcccctctggccgtctgcctccacaggaggcttgcaggtgcccacatcagccaacgtggccctcggtggggctgtgcttgccttcttgccagggccactgcagtagggaggagtgcagagcagaaacaggtgagctgggctgaattttctgcttggctaattcagtgtggcttgactccaagaaggacacaccgacctccccatcatcttgtttgttcagccttgcagaagcagttttatgagaaaccattacagccccggtggtctgggcccagacccggtgcacaccacgtgcccgcactggtgcggggggaccattctcggtgaatatgatggatgcacaggaagccgccctgccattcagtgagagctcaccatgtgtctgcccctggggtggggtggggtgggcagtttccagcctttgcccatgggatagagctgctggaagtctccccgagctgaggaggcagagctggggtggctggggctgggggttgccatggatacttcctgcaagtcctgacgccgctccttcctctttggggatctgtctcccatactgtccctgctgcctttacatcttcagggtggagagggactctggccatcctggggccaaccatcctgtgtcactgctggtgtttgtgacctgcggtgggccctccctcaactccgtatctccagctctaagccagagacaagaatatcctctgtgggggggtcccttcaagggtggatggagatgaggcgttaggtgtatccgatgctcaggaacggccccgcacctgctcatctttatgatgagcagtgggactgcgggcagagggagccacacgcatccatcctggctctcagcatcccagggaaagatgttctgctctatcctgatcagcctcgccctttaaccaaccacaggctgcctgcagtgtggccgtggggagctggagtcaggcatggtggcagcccttctagacagtaggcagtaggtaagcctgctgatcacggagccgagattctctgtgggacagagctggtccccagcatccctgtggcctttgggccaagaactcagtcggctactttgcttccaggcttgggaacactcagggtaggctgggagtcccctggtctcaccctgtgagccccacataagcctgtggatagcaccagctcagcaggtgaccccctcatcaaaaccccaaactgggatgcttctctggctacataggcatggccacatggggacagtgggaggacatgtgataatttggggcagcggctgaaagcctaggggttagggctactgtgtccttctaaggtggtgcagggcgcacagccctctgggcctcagtttcctcttctgtgaaatggggactctatcttggggccgcaaatgccagtgtcttctctggaagaaagggcgactgctgagggaagcagcacacaggtgtgagggtccaggccccagacgggatcccacaaagacctaggacagtgagccaagagtggagagaggggacgagggtggactggggtgggccccaggagctggaaagtgaggaaaatccagctgtgtcctgagggttagactccactgccagtgttcacaggatctggagctgatggggacctgcggtgtcaccctgaagggacagatggcccccaggctagcaggaggtggcagtgtccgtttggcagcaacatttgacaagcagaaggcagttggtccctcctgcttcctgtccaggctcttggggctgggaccccactcccagccctgtcctccccaacctccccacacttacacaggccactctggggcagaggaggggtgctgtgatttgtgggtttgggagaagttggaagcataatgggtcaggcctgcagctcggtccacactgcctgtgccaggtggagcaggtgagggcatccctggctctggggtggtgtcactgttcacactttgtcctatagccaggcccttcttgggggtgagggttccgtggagccctccatctgcctggctctgccgatccaactcttttctctctcttgggggtttcaaacttagacaggaataggggtgtcatttattgggccccagacaacctgaccaggtccctcagagcactgaggccgggaggaggagggtggaaggagatgggaagagtttcctttgtcctctctccctggccatccccaaacctccacacaaacctggggtggctgagcattcattatgctttgtctttgtaaataggcagctataaaaacctatcagcttgcagcaccttctccataacacaggctggatggatttataacccaggtcccctccccgagagaagctggcaaagcagaccccagcccgcgctggctgccatcaccctccctgcccctgccccacctcatgcaagaaacagaagggaaagcacattgagttgtaatatgttttcgatggaatttgtcacaataagaaactggattttgttggggctcatgggatgtttaggaaagagccagagagtggtgcaagctgtgggccctgccgagaagcctgggctacaggagggcaggggctggagtgttggcagggtcgcacagtggctcatctggacagtccacagcggatccagcccacactgtgtcaggcactttgctgggactgggggatgtggctgtgggtacgattgacaaggtctgtgtcctgaggagcccgcagagcagatgagatggacatgtggtcagtgatggtaccgtgtcgggtggaagagacaataggctgagctgcccagagcatcgcctgaccagcttgggtggtggcacgtccaggagggcttcctggaagaagtgaatttattcaacacatgttcactagagccagtgatgcttaggcactgagagtgttgccagggatacaggagagaatgggagagtccctgagtcattccagactgtggggctgaagtgtccgccgatggaggtgtgggaagggcacagcggcttcccacgtgagcaaggagctatgcaatgtggcaggtggcagggccaggcggaggtgcctccacctgtctccagaccccaccccctacccaggtatggaattgttgtctccagttggcagagaaggaaactgaaatgggggtttcacctctcaggaatgggtaggccaggattttaacccaggcctgcagacaccaaatctatccctcgctcaggcctgcactgacctccgtgcacctctggggctccaggcagctgcctgggtgggtgctgtgtctggggtctctcctggcgttccttaggcccctcccctatcaccgtccttcattattcacttggatgccttgatggtcggggctggaaccccccgagctgacccaccatgcggctcatcttccttctccttccagtgcttggtgatcttgagagtgaggctgaaccgttgcttgatttttctgtgacccagatgaagagctgggtaaccatttgctcaataaagtgagagaccccatgttctggttaaagtggaggcactgaggaccagcgaggggaaggcagtacttgtatttgtcagcctggaggagacgccagataccagccagagcaccccagcctgtatctcgaccaccacctgcagttggtgctgaaccccccactccaccccatagatgagacaattgaagcccagagaggccaggcttcttgccgagggctgcacagccggcagggatgctggaattgggatttggccccagccttgtctgactccaaagccaatgctatttccaccatacccagtgtctcccagagctaattttgcggctggaactgcaacccgcaaagctatctaggacaggcaactcgatgaaagagaattaggagggaatcctagaaaaatggggctcggcagctcccggggaagcctggagaggaggtggcgccgaagcctctgccagcagattggggtggggctgttttcagtcctctctggcgaggtgttttgaagcctcctctgggaaccgtgtgcctctgtccaggactggctgtctctctggaaatcataccctggcagcatttggctttgggtgaaaggagaagagaagattctggccattcagagcaggcccttgtgcgggatggaacccattttccagaactcttgggacagggaccagggtggcaggcaggggcccgtggactgcctgggggacctggtgcttggggacttagagatttgttttcctgctgaatatattgctttctcgtgcctgctttgtgcaaccacgtgaggatgtgggggtgaggatggccgacaggacacgggagtccctcccgacaggggccaggcggcggcgggggtccgcatgtctcacgtcagcatggctctgtgttttcactcctctccagcacatatttagtggaaatgaactcattttattattaaaaattaaagtcatgcattcatagggtaaacaagattgagagcatgtggaggtgcactgtgaaagtgcagttctctcggaatgggcacttagagacgcgcctgttctctgcagctgccgcaggggtctcatcttgttgggacagaacacggttgattcatgcaattggctgttgatcaacatcaggttgtgtctagttttgtttttttcccgtttcgcacggtgctgcgaattcacagctgtgccagtgtatctgaaggtaaatcccacgagtgggccttggagagtcagaggatggggccttctacgtggacttggtgtggttgggtgtgtgatgcctgcatggggctatgtgtttttagcccttccttctgacaggttctggaggcctcctctgtgcctgccagccatgcagccgctgagccgagcatcacccaaggcttgcctgaacctggcctgggtcccaaaggaacactgctctggggcatggaggttggctggttgagaactaaagccacatcagcaggggcactgcccccacctgctggggtcagcccccgcccggagttcagcaggacctccgtgagccttcgtgcaggtggctcattgcagcacgtccccttggggtggtggccattggcttgtggttcctttgctcactgcggggaggaggacagccaggcacaggtgaaaggggcttgcgggtgacgattctagtccttggcccggggaatgtccctgggcttctgaggcctcacctccctgggtagtcaggagggttacgagggtggggcctggccctggggactccagggtgtggcagcggtgggagtgaggaaacagccctgagacggagggagagaagggcgatccagatggcggtggcctcctcacccctcggccagtgatgcatggtagtggttttgacgggctgacctcgagggtctgcctgggagccgcttggaactctctggaggtggggccggccctggtgggggcaggaaggtcccagagcagcttgttaagtgggctgaggacaagtgtcaggagacctgggtctggatcccgctccaatacccctctccgtgtgacctccaagggatccacctgccttggcctctcaaagtgctgggattacaggtgtgagccactgtgcccagcctgtgcccagtgtacccatcagtaaagcagggatcaaacagttcttaaatcctgcagcggtggtgagagccacctgaggaaacgatgcaaagggctttgacggagtctggcacagagaacgcacccaataaatgactgccgtgacgatctttcttctcgccctcaggtggtctctggaagctcctctgtgcggggttttctcatttgccagctgtgcatcccccggtcgtagtgcggctcccacgggggtgtaccaggagcctctgctcctcctatgcttcctgaaaaagggcccagagaatatttccatcaggaataactgagtgaatcccagaaacttcctatcacatttagggtgattaggcagatgcatacgattctcactgtgggaaaggagctggcgacctcgatgggttgtggttcccgcagggatgtgcttgcgctgctgttactccagccgtagctgaggcacgggagaaaacggagaccccaagaagttcagaggcttgtgcaggtcacgctgttcctacgaggtagaccctgactttgaccccaggctgtgtccctgccaagcttggagcctctttctcaaaagggcgacggaaggatgttgttacagatgtggttgccagtttcctccttttcattaaatcaccagggaaatggtctcttgcaacccccctaaagcagggggaaggaggaggacaaaggtcaggtcaccatctttgctggcatgtgagtggggtgggggtgggggtggggagctaggagacctggcgctgggcccttgaaatatccacatttccacaacattctgggtgtcagtgagcccctgccttcctccctcacatttatccggagctcttcctccgcagggaagaacaacagcccgagatggggttatttcaaggggatttccatggaaacgggagggtgggaggttcctcccagcacttgtataatgggagttggctgaggtggcagcgtgtccccacggaagggtgcgagggaccttctctgcaccgcaggcctcctcagagtgggaggcaccgacccgagagtggctggctcccctttcatgctcccaccctctctacccagctcaagaccccggggctccttggtgtgagtgagagccaggccagctccccagggacccccaaggcctggtccttcccatggtctcttttctctagcaggtctttgtcttgggctgctgccagccacagcttcctggcaggacctcctggcaggacctctgtgctttgagccgctgttgctctgccaagaccttgccccgcaccgtggtctgaatcagcccagcaccccttcgcctctgttgcagtgctcacatttatccctcactcctccatccagcatgttttgttttttttttttttacaagcagacactttgctttataaaagaattctgctgtgagctgccgtatcctctctgagcctcccttttgtcatctgctgaatggtaacagcagcgcctgccatgcctgcttggtgaggattccatcaagcagggagacagtgggccgttggcggggagtctgagcaggtgtaccagtatttccagtcagctgatggctgatggacatgttcttggaggcagggaactcggaggcctgcagacgtgccccaggatgacaagattcatcagttcctacaagccctgcctgggcctcatgcttttcagtgtgtcctgggctttcccgtgtgaaatcttacctgatttttatgccaccttgagaagagtgatattcatctccgttgtacagatgaggaaactgaggctcagggaggcaacgtgatctttgcaaggatccgtctgttccccccgtggcctggctgcccctcctggcagtgcaggtggagttaaaaccatacaggagttaaaatgagcctcgatgggggtgggaagctacgactggaaaacgtccgatgctctcccaagtcaaattgtgcttggtgtctgtgggtgtgtcggtgtgggggagggaagctcagcccttttgaaaagtggggggtggtttgacgacgctgcaggggcagctccgagttctagagtctcagaacgtggttctaggcggttcatgtggatcaagtgctgttctgagcactttaatatccagcgtgaccataaggataagtgccactgttactggctttttcacagatgaggaaactgaggcacagagggattaggtaaagtgactggagtcactcagccaggatgtgaatcacagcccacacccatgtgcaccaggaagccttggctttcagggtccttggagggtgtgccgggcagtcgcctaagctgggaaaccttgggcttgtctccaggccatgtggccatgtgagataggagtcctcctgtgttatgttctgcgacactgtgggcagagggctgaggaccccagccctcccttagaacatcatgttggtgtgagacatttagagccaggcctccctgcttagaaagcacctcttgggtcgcttgcattagtgaagttatacatttgaaactccatttatttatttatttatttagagacagggtctctccttgtcacccagactagagtaccgtggtgccatcatagctcactgcagcctcaaactcctagactcaagtgatccccctgccggggcctcccagagtgctggcattacaggcatgggccacagcacctggctgaaactcccttttcatgaaaagaaacagcttcaactttgcaatctcatctgtctgtctatgaggctgtgcctttgtgtgagatgagagcagtcactgtcacttgctctttgaatatttgattaacaggtaaacagcctgaaatccatttgacatcttatccttttgcaaacttggctaaattctcttaaattggttccagttggattaattaaatgcatggttgcttatacatgtgtgtggaaatgattctggcaggtcatgtcttagctagatagtgaacataagcgtctagaatattctcagctgttgcagagactgccaggaatgaccttgaaaaagtttgggagagggtttttttttttgtttttattttgcttttgttgagacagggtctcactctgtcacctaggctggagtgcagtgatgtgatctcagctcactgtaacctctgcctcccaggctcaagcggtcctcctgcctcagcctcccgagtagctgggactacaggcaggcaccaacatacccggctaatttttgtattttttttttgtagagttggggtctcaccatgttgcccagactgatctggaacccctaggctgaagggatccacctgccttggcctctcaaagtgctgggattataggcgtgagccactgtgcccaggctggagaggtttttggatgcactgggccatggatgtgaaggtgaacacatggaaacgatccctgccacctgcttgtgtgtccagtggacatgtctctgatctatccagattgttacactgtcaaagtgaaaactgctgagagtagagccatctgcctggccaggcatcgcttggaagcgtgaagacactttgcctttttgtctcatgattctctctccatgtgcagcttcgttggcttaaaagaaattaagaaactgggcccccgcttaggacctgctgaagtgcagagttactgtctttgaagtggtggggtagggaaaaataggaaataaggggtctgatcattttgagaaacctcagggagatttacacctgggctgtgcgaggaccccggagagtggcagagtgtatttggaatttccagtagtcctcattcctcccttaatatccaggggatctggggcctcagtcttcttatctgttaaatgggacaagtaacgactaggctttggggttgtcaggaagactgaataagaaaatgggtatgaaaacagtggtcacggtgcctggccctccatccctgtctccaccaggcctacctgtctggcccaggcctccctgatctccgcgggagcagacctcctgtaatggtgtcaaaggacccttgttctatttatccatctgatttccattttcggggccactgcctctagccatgttaggcacatggtgagtgtctgtcccatcaatccttgtcgattctgtggtcctgggtgggccatagcgtttctaacctgtccactctctcctaatcaggcatttggacctgtttgggttcccaaactctgtcacgggcagagggctgcaggaggctactcacgggccagggttgtttggacctggttgggtttccaagctctctcgcagtcagggggctgcaagaggctacacatgggccagggttgggctgctgggctgctgggctgctgctgtgttggagctgcctagcacttgcttcgttgctgcacctgagaggctgtgtgggctgagacagccagaaaagatgcaccgggagccatctgtttgcagcccttggaccagatgctctgcaaggactccgggggggcggtggggtggggagggaatacatttgctgagcacccagcatctttcagagcctcagcacagccctacaagctgggcattgccatcatgtttatacggaccaggaacacgaggctcagagtgactgagtcactggtgacagtcacccagccaggaagtggcataggtggggcttgaacccagggcttcctgagtcccaggctagtgttctttgcctcaggctgctgaggttccagctgaatgttgcggcagagtgacttctgagaagtaccacggaagggggtgactcacgccggggtggtctggcttctctgcccagtgcctgaggacacccaggtccctctgcggccttggggctttacccagcgtctgcatggcatcccgcagcaccctgcctctggagcgcaccctgtgtgtatcctaaagtgcgctttgcctagaaaacctttctaatgaaactggtggaagatggagaagccaaattcagttttcagagatgacactaatcctattaaggttgatggggccagagcatgtgtggaattagtcctgccaggcggcggccgggcacctgcctggaaggctggaggggatcctaacaagagtggtgccgaggagagagagggaaggggcctcatctctcccagaggtttaaaaaaactgaggccactgtagagcttggttctcccaggttcctggggtggaaaacagggcttcccacagcagacggaaatgggaggtgggcagtagtcaccgagcagggaggctgtgaagtagtcatttaggtcgggaaagtcacgcaagatctccaagcttcagtctccttagctgtgatggggggaatgggtgccccccttgggcgacttgtgggacccagaactcatcacgcagagcgtcccactgctgtgcagccactagctgctcggtgctggggctgtggtatgagcatggactctggagtcagaattcctgccttcaaagccccactcctcaccttgctagctgtgtggcctggggtgagttgcgtaacctctccatgcctcagttccttcatctatgaaatggggagactgaaactgtaccccacaccctaggggtgttggaacttaagtgagttaattcatataaaacacggagtgagtgcctggtacgcaggaagtgctcagttcctcttggtgcctgtgattattcccataatcatcactggtgtcaccttgtcgcctctcccagcccttggggccatgctatttgtggtaggaaatggggcctgaaaccatcaaactaaactggactgaatgatcagtccaccgaccaccacagtcacggcttggtggccccacggactgaagcacgtggccaagggcagttttcccttctctgtgtgggttccactgggtccaagtacattgttcctaagcccaggcctctggccactcacccacctcctgctgagagcgggcagagctgatgcccctctctgggcagaatcagcccacggctgggaggggaggccaggcctgctgctgggggtgcagatagtggggagctgcaggccagccactggaaacctggcctgtgtgctgagacagcacattggacacagtctggtggccttcccatagatcaggccacagagtcctcatcctgggtccacccaaggcactggccatttccagatcaaagagcaggtggattccagggtgagacggtcctctctgctggctgccttcccccacccacggacaccgtttggctttgatggggctgtgtcctaggcttgacccaggtggtcaggagccttccactatgcagtgggatatgctgcaggagtagggcggagaaggaagggaatggccggtggtgaacagctgccatgccccaggcatttaattttgaccgcagtgctggggggccatacccagtaatacagatgaaggactgagactcaagggtcagtgcttgctcagagacccacagtgaagagtggggccatcgtgttgcatgtgtgtggctgggccccatcccctcctttctacagcaccacccctctattccttatcaagttcacctttaaggctcctttgcccctcactggggcagtcatgaggggagcagaggcctcggatctgggcatggatgagggtagagccctggctgtgtcctgcaggtgacacgtacagagcagaaggtgatgctggaaccatccgccagggaagggctgtccaggaagaggtcacagcctgaacagaggcgaggtggagagagagtgctcgtgtgtggggactggtggcagctcaggagggcaagactgtgacttgcaggctgggaaccagggaggctgcaggtgatggaggccccatgggtctcatggggcgggctgaggagcgtggctcctcccctgcagctggaggggccaggaagggctttaagccatggagtgagggaccagagttgggtttacaagggtcactgtggtgtcggtgtggaggggcttgaagggaggagtttggagggaggggctgtagcaatgttgagattccctaatcactttagtgttttcatgcatgagggccccgaggtgcttttttccagagcctgcactgaggttccctgttggcggcttccctcaggctgctggaacccccttttcctgtgcacctgcagagctggaggttactggaaactcatgtcctctgccagagtcagccctcacccagtcactgacaggtgcagtggtataaataccccagctccctccctcctgatcaggacactttgagatgggacctacactgtccccagagctcccatgggactgagctcaagtcgcactccttgagatttttcctgttatcacaccccacttggcctccttcatgtcctggccccacttccctacacccttcctggctttcccagcaacacttcctaatcacttccagaaaaatgttggcctcagggtctgcttctggagaacccagcctagcagagggcagcaaaggtgttgaggcactaggagaaacatggtgagatacagactcaggcggcgtccatcagctatgccaggaggctctgagacgtgccagtcagggagggaggtgctgaggccacagcaaactgggggccagaagtaagcaccacagacagcaccaaggctgccagggtccatcatcttgggtctcccaattggttgggtccagagaccagcctgctggggtttacagagccagcatgttacatctctctgtgtccccactgctgaaagcctctggtcagtgccatggtgctgagggagcttgggcctcccttagaggttgctaagagccccccacacctgccctgtgagttgttggccccaggggactctgagtttccctgtttctggttttcctgctcatgggattgggagtctgacctgagcttgctgagacagataactgatcattcagatacaaaactctaaaggttagaactcttttctgataacttaaatagaaaatgaattcatgcaacatgtactcatctgtccatctgcgcatgcatccaacatgtatgtatctcagttgatggttgtttccatgccaggccttggggatctagagatggcttggtccctatggtcatacccatgtgaagggagatagaggtggacagaaagacaaccataatgtcattttgacaaattctgggaaaagatgggccacccagcctgtgagacctgtatgtagtgatggagggatggggaacggtggaggtgtcagggaagctccccaggggagctgttccttgaggtggttttaagagcttagcaagagttttctacgcagtgaagtgaagacgtagggcaggtggagtggagggttggcacctaaaggtcctgaaaaggaatggtgttgcaagaggcagcaaatagcatggggtgggctaagctccagctggttgagtttgggtcagaagtgtggacttggttggtaaggactcctgagagtgtcatgtccaggcacttggacttgatgctgaaaatacagccattcaacagagaagtatgtatgtctgagtgtgatgtggttagatctttagtttaggaagatctctctgttcatggtggggagagtggatgagatgggggtggactggaggcagaagagcagaggagaagagcagactttctaggagacgaagcgggaggggctgctcccagggcctggctgtgagtggtactgaccagagccatccatcagcccagatcatgtctccctaatttagaagaaaatgagaggaaagagccttttcatctctgcctcttctgggatggaaatgagctattttgaatcacgctaaagttagatttctagttttccttttaatccgtgtctcatttagagcattagaaactggaaaagcacccccgtatgaatgtagtgtcataaatcagtgcacgtcacaaactgcagaaggagtcatttctacaagagtgtgcagccttgcatttatgataaatctaatgctttatctttttgggcttcagactagagtaattagtcatttctgctgatactggcaaaccattgcatgtgcagtgttaatggagagcaaatgtatttgaactgcttttgcaattgcttgtgagcattgctgtgtatctcttctacctgtttctagaggggaaacatcctatatatttcattcttgcctattctaggaataggtgtggtaatagtatcaggtaacacttactgggtttcttccacatctctggcactgttcagagattcaataagaaaggatcctttgtccaaatggaagctgccgtgtgtgtgttgaggtccttgggtcaatctgatcatgctctgtctcctgctctgtgcccgcactgtgtcagggaccacctggataccactcatgtattattcacctaataggtttctttaaatgttggccctctaccataaatggaaaaccagcaaatcaggccaaaaatagaagtaaccaaagacaagcacaggtgtttctgctctaatgaaatagagacatcctgagaaacttctcattctgcaaaatcccaaacaaatgacatcagagctgatggacagaatggggttgggacagatgactggaagtctatgcagtgtgtcaccagacacccataaaaatattaagttttagaaaaatctggcacaatgaaagaaaaacatattaattcctgttaaagaaacattccaaccattatacaatcttatcttgaaggaaaaagtgaaggtcatttaatggaaggggctgaaagggagggagctgaattatggctgggacatggcctgggtacagagggagacccgtccaccaaactcttccaaactgagctaagaggtaggtgcgcagggaatgagtctattctcttgggccacattcctttgaggcttgatgttcagccattagtgtattttgcattcagttgcttcctctggctggcacaaaacattaacacactgagaaaggctgcgtttgaaccaacctaacttttacattatactgacatcattcccctatgtacaaattgcatacgacctactttgcaaaggcaatggtcatagtaactgtacgatggtgagtggggttgttaaatgcagaccagatactgctgtaatagggctgtgactcaggactatcattccagttgcctgatatgaaaaaggacatgaaggagaaggttttatgagaactctggcaaagacccatgaaaccactcttctttgccctccagaaaacctggtaaaactgaaagtaagccagcagccccacgggtttgggattgatccagaagactgaaggaataaaaacaagctaatatatttctatcccctcccttctaaggcctgtagttggttccattatgatgagacagcagccgatgcacgtaaaaacagaggccatgcatctacacaggggtgttgaggattgaatgagattaggtctgaaagaacacacagtggagacagtgttgtcattgtggggacaaagatttcttttcacaatttgagtgacacttgtcatttctataggcagttaagagacaataactgcatactgttggccgctgtacctttcctgtgcactgagtccctgagcatattcgtgagtctttaaaagtacattctagtcaatgtagaaaacagatagtaagggacatgactctacctagattaagctcagttttgaagcagggcctagaggatttagaagtgacagagtcctggattaggtgacatttgataagagctctatccctaacttcctcttaattctggacaagccactccccgctctggctgtagttcccccatctttaaaatgaaacagttgaatcagacaactacaaaaggacttgccaggccttctgtctcagatcctgtgttgttgaagggatgtctgaggccagagatgcagtttacttctgagcaattacagttgtatttcttgtgttcagcctagccctttaccaagctttgttgaagaaatggagcaagatccttgtcctttgcttcggtggtttttaaaacttggatgcctaaagaaaccaggaaggtcatatcagtgagcaaagtacaaaatgtataagcaagacacagtgcaccatgacgctgagagtggcaaagtaacagtccaaccatgcatcagggagtggtggggactgtggcgtgctggcaagcatagccttgtgggattctgcattggacagctcttacaatttctggagagaaatcagaaattcaggtctacatgttgaatcttttaatattgaaatgttcacttggcaatgtgtaaacatcatttgggtcaaagcccatctgtgaaccaagttagtcaaagacaacttgggcatttggacctcagctttatagtctatataagggttggtaaactatggcccacatgctaatctgacccacctcctgtttttgtgaataaagtttaaatgaagatgcagtcacacctatgtgtttaggtattgcctgtggctactttcacactacaacagcagggttgagtagttgtgacggtagttgagacagtgtggccctgtatttattctctggacctttatggaaaaaggttgctgacccttggttcatgtcaatgactcatatggagcagcaggaaacattctgaatttggagctgaatgaccagggtttgacgcctggctcagggaactctagtaacagagtagagagtgtttctcttttctccttttggaaatgatcctgaagcaacaaatggaacaaatggaaatgcaaatgccatctttgatggaaccatagcttctggaacctcatatgtggggaagtgcagtaaaggtcaaaaatgggtgcaggatgtctccaaagggaaataaaatgcccatggctgtaaagctcagacagagcaaacaaagcagaattttctacaccttctgtggtgggctcagatggcaagacctatagttcctctcatggagcaataagaacaagtgcattggctggtgtcttagtctgttcatgctgctatagcagaataccatagattgggtggcttgtaagcagcatacttttatctctcacagttctggaggctgggaagtctgatacccaggtgtaacagatttggtgtctgctgaggacctgttttcttgttcatagatggtgccttcttgctgtgtgcttgcgtggcagaaggggctagggagctctctgagatctcttttataagggtactaatcccattcatgagcactccaccctctaagacctaatcagttctgaaagacgttacttcctaagaccatcacattgggggctaagatttctatatatgaatttcggggggacacacattcagaccataacaactggtgtgggagccttcccagatctagttttgcacaaagtgaaatcagggaagtctggggtgaacaaggtctcacatggacaagctatattttcaaaaagcagtctactagagaggcaaagtggagaaaaggggctgtgttatgtgccagcctgtggcatctgatcttaacaagaaagaagtaaaggccaaaagaactgactctcactcacaagcctgttttctaagaactagattactcttttggacactctggtgcacgaatcttgtataaatagatggttcaggaagaactcatcacattcagagatgagtaataattagtaaggaccagtgcaagctctatctcaagatcctccaaaaatgaagaaagtaacagatgaaaaatatgcgcaagaaaacatttgccataaatcagatgaaaatgtcaacagtacataatgccttggactaaggaaaaaaataattgagcaatcacacctctaaacaagagtataaagttgagatacaagaactcaggagaggtggtaacatgacagaagagagtgaaacctgatatgatgagctccacaaagtagatagattaacaaataaacccatctaaaaagtgaagtatgcaatggaatgattatgacagggaacacagaaaacacagtaagagatatggaagatgagattgggaaaagcaagataaattaatgtaaagagagagagagaaatattagaacatgatacagaggacagataaggggatccaacatacacataattggtgtccctggagataagaatgaaaataattaaataaaaaaaatttaaggcacaattccagaactttctaaaaataagatttgaatttatggactgaaaggaagtacagtgagtggctgagaaaatattgatatggtgaggtcaacagaaagacatagagaagtactaatttcctcataaatcaagaacccccaaaaaaattcaatagaagaatggataaaggatgtgatgaggagatggtttacataaaaggaaatagaaatatcttttaaacacataaaaactcaacctcactcataataagaaaactccaagttaagatactgttttttatctatcgtataggcaaggactaaaatagctgatattaccatgatggtgcaggtatgagggaaacaagaactctcagaaacatggctatggatttcacttctaatccttgtctgatggaagtacacaagtaagaggccaggtgtggtggctcatgcctgtaatcccagcactttgggaggccgagattggcagatcacttgaggtgaggagtttgacaccagactggccaacatcacgaaaccccatctctactaaaactacaaaaattagccaggcgtggtggtgcatgcctgtaattttaggtacttgggaggctgaggcacgagaatcgcttgaacccaggaggtggaagttgcagtgagccgagatcacgccactgcactctagcctgggtgacagtgtgagactctgtctcaaaaaaaaaaaaaaaaaaaagaaaagaaaaaagaaatacgcaggtgagaaatgacatttatccacatttgtcatagcattgttggtaatgggaaagaatggaaatatctgtaatggccatcacaggggactgtttacataaattattccagagcgtgatagtggatagcaatgaaaagaatgtgacagctcagggtgcgctgatgtagaatgatgtttaagattctttaggtgaaatgagcaaggtgcaaaatagtggatgatccatgacatgctttatgattaaaacatggtatcttatgactgcttgtgtgtagactccttctggtaggatgatgcacctgaaagtgctcatagtggttgccactagacaggagagctgagtgagtgggagaagggcctaagggagacttattttcactgttagcttttttgtgccctttgcattttatactcatttcttaaccaaaggctgcacagcttaggttgtgaccataagctctagagtcagctgttcgagttcgaaccctgactgggctgcctactcgctgtgtgaccttggacaagttacttcacctctgtgggtctcagagttgtcagatggatgtaacaatggtgcctatttcataagttgttgaggatgaactgagtcaattcaagggaaagaatgaggacagaacctggcacaaaaaaatacagtcaaattagctattatagtgactgcatgtatagttgaacccagggtctggatttggaacccgtatgcttattggcccaatgggggggatgacttggccacagaacagccacagccaggcagtaaagggcacagggagggggccttagcctggggcttcagggaagctgcccaggggaagtgatgctgctgggtcttacagaatgatgggaaaacttggcggcaccgtttcaggcgacgctacttagatctgcaaaactgttgatgtcttacaataccaagtgtagggcggggctggagaaatggaaactcatgccaccactggaaatggatatgaaagcaaaccatttgcaaagtcatttgccaatatccagatatgtccaagcagcccctctgttccccttctaggtaaatagcagggaagccttcagctgtgcacagagagccattcagcaacagagcagaccctcgaacaacacggggttgggggagccaaccccctgcacagtcaaaaacccacatacaacttttgactccccaaaaacttaactaatagcctcctcttgattggacttccaaaaacatgaacagttgataaacatacattttgtatgttatatgtattatatactgtattctaataaagtaagctagaggaaagaaaacatcattaagaaaatcataaggaagaggaaatatattttctgttcattaagtggaaggggatcactataatctttatcctcgttgtcttcatgttgagtaggctgaggagaaggaagaggacgggttggtcttgcagtctcaggggtagcagaggcgaaagaaaatcatgtataagtgaccctcacagctcagctcatgtttgagggtcaactgtgttctaaaatccaagaggctctgaaagcctaaagattttttcctaacttatttggcagtgaaacccaacctaaattgatgtgaaaccatttatagcccttctctatccctgtagtgtgaagtttcatatgttatgtacagaaatagaaatgcgtatgcgtgttgtcccagaagcccttgggggtgacagatgtcctaggtgacctgtgcaccatattactgtacagtccaaatcctctatcctgaaatactgggcccaccctttgggatttgggagatgacagtgaagctggtgtgtgttcagggaattccaggttgttgaaggtgctggagtagaggggagagggggaaacgaggcagcaggcgctgggtcccgacggtccttggacacctactgaggatgtgggtacaacacttctttcatccctggggctcagagatgtctgctgaatcagtcgaggggccttcagtccactgtgagccccagaaggcagagctgtggcctcctccgacttgtttctcagaaaggtcacttgggcagcaggtgaacatggctcaggagaggcaagggagggtggggacaggtaggaagcagaagccacttactgggcaaagaccatagccatgaggatggggagggactgagtagccagagaggcgctcatcaaagcctactgctgtcgtctgaaggtctggtttctcccaaattcgtatattgaaattaagccttaagatgatgtgttaagaggtggggcctttgggaggtggtcaggtcatgaagatggagccctcatttatgggatcagtggctttataaagggaccccacagtgagaagatggccatctatgaactatgaagaggccctccccagacactgaagcttctacacctcgatcttggacttcccagcctctagaactgtgagatgtaaatggctgttgttataagccccccgagtctgtagtattctgtgggagcagccccaaatgactaagtccacggcccatcctccacccactgaggccataccactgggaagggtatcagaggcagggttggggtcttagaggtccagggtctcttgtccaggctccccagaggcaggccccgaggtgtggactagagtataagtggtttatcaaggaagtgtacccaggggaggtccagatacaagtaggaggggctcaagcacagaggagccagggaagggggggctctggaccgaaaccatgtcccctccacggctttggacttagccccttggggacacagacgtggctggggatgggtcacaaagtaaccagggtgactgccagcctgaactctgggctctccagacttctggggaccgtagtctcagcctgagcagctttctccaaggagggtcctggaaacctggggcctgcccctgattttgtcctgggagcagggcccctccatggtgttctggagcccctcatattagcttttgaggttgattgcttaattttcagtttggagagccagcagacatcttgttggtagtttgaaattggccagggcaggggtatttataccatggaagctggcaggggctgccttccagaacctctctccctccaggtgcagggaaacacggacctgcacaccctggtggctttcacaccttcccctcctcaggcggctcgtccccttgctcagttatactgatccttgggcttgcaggcagaaccatctctgtcatccagaatctccctagtaaccccaaccccaaaagtgggctgccggaagaggcagatggcagtgtcccagggctatggaagccactgataaacatcttccaacaaacaggatcagggatctgcaaagttttttttttttgttgttgttgtttttttgagacagagtcttgctctgtcgcccaggctggagtgcagtggtgcgatcttggctcactgcaacctctgcctcctgggttcaagtgattctcctgcctcagcctcacaagtagctgggattacaggtgcctaccaccacaactggctaatttttgtgtttttagtggaggcggtgtttcaccatgttggccaggctggtctggaactcctgacctcatgtgatccacccgccttggccccccaaagtgctaggattacaggtgtgagccaccgtgcccggctggatctgcaaagctgttgaaaggaggagtggataggattacaaagttggcctgattctgctaaatgaacatttcaacatgtttcccctcgtgcaccgaggaggcatgcactctcctctgagggtctgaggctttcttcacggagactttgtaaaccacttctccccagcactctggagtgggggagtcacgtagggaagtcatttctctcctcttggacctctcagtcccctttggcttctcgctgataccaggcagtcaggtggatcagagaagacatggactctggagctggatgtctggtgcaggcagtgtggcttcaggcaacttgctttgcctctctgggcctcagcttcctcctggaaaatcatgatgctcatacctagggtttcatgggggtcaggcaggacagtgcaggtgcaggtatgagcaccatcccatcaaggacatagtcctggctgctttcagtgatcttggtgctggggaaaatggggctgattctcatggaagaggaaatggattcatgtgcccacttcttaaatggaaattcaaggccttgatcgtggcccacttatgaggtggggactagtgaggcttcaggtggtgtcctaagattagaaaactggagcattcagttgggagtcagacctctgctgatgtgctgtgtgacgttgggcaaggtgtgtgacttctctgggccttgtgttcagtagaagcaggtcatctttcaatagacctccttcccagaccctctcggatgctgtgggcatccagctggcccccatgccacacctggcttctcggggactggcctgcattctcacacccgttctttctggcaacgatgggcatttgtttctcccctgccagcaaactcctttggaccaagaattgatgccatggtgttagcatacaataaagcttcaaggagctggtgaaacactctgagacggtcataaaattggcttttcatccctgtcggagctttcttattacagacaacaagcagtgtcagggagcagcacggtagatgctgcttaacccagcagcaggcgagactgagggggtctcgtgctgtttgtcaccaccgcctctctgagatcatccccaacccatactgttgacattcactgggatacctggctacctgttttgggtttccttacaattttttcatttgtttaatagtttttatttttcaaagtcttaaaaaacacaagtcagtgaaagttttggtctttgttactgtcttcctaccaggtacgagggcctgaaagtcactgttaattgattattgattatctgattagaaccgtacatttactttttaaggaaatgtgaaaaataagcctagaaatgaaatcaaaagtgcggtgttgaagttagaaatggcagcgtcacggtcaggaattaaagctgctgcttctctgtcacatggtataccacagggggtggtagctacagcccatgggcaaaacttggcccagtgcctgtctttgtaaataaagtttgattggaacagagtcatgcccatgtatctacacattgtctgtggatgcctttctgctataatggcagagttgcagagttgccaaagaggacatatgtcccacgaagcctaaaatattggctatctggccctttgcagaaagaatttgctgacccttggtatagaggaaagttagtggcacaagtttagatcgcctatagtgaaagaccttgcaggaccaggagtcaggcagacctgggttcgaaacccgctctgccacttcctggttgtgacttcaagctgcttgccttttcctctcagggttctgtttctgtctctgtcaaatgcagccaaatactgcccagtttgcacagctgtttttggagacagagacactcagcaaggggtccagcccctggtgggtggtatcccaatgtcactgttggcatttgcagtgtcagtgatgtctggcttattcatcaggccctcttgagtttgccccaaacgagtcaaccctccagggctgtgaaacttctgctgccttgatacgatgtgatttccgaaccagaaaaatggaggaggccctggcccctaatgcttaagtcacaggtgactctgtgcaattggtttcttccctgtctcctgtccctctaaaaagaaccacctgagctcccggactgtgctatatccaggcctaaaggctggtggaccaggacacagtcaccagcaatcagctttcatcgacttggctggaccttggaaacttaaggcttgccctgccctggcttcttgcggtgagcaggtcttatggagttgctctaaccactgtccatccaggccggggttagaggatccaaacacagccatgttgatgggaacctggaggcaaatagagggcatgagaccctcctctcctgaaacctttctggccttcttattggatacccaccaaacagtcttgatcttaaaactttgtgcaaagcaataggatgaacttgtaggacatgcataaagaggaaggtatggattgtgacatgaggctcttagggatgctcctcctacagtgctgagtgaccctcctcagaggcacaggtgtgggcacagcttgttctttcaaatcctggcaccccagctgttctgctgtctagctcgttccctcattgacaaaacaggaataacagtgcccacctccttgggctgttaggggatcaactgagatgctaagtcatccaggagggggagcctctttcccagagtcctgcctgtgcccggtggctgtgcagacctgatacccgcagccatctacctctacacctcatgtgtgttagagaacaccactctgatttcagcctctcagactgggggttcagtcactggctgctaaaagcttcctaaagccaaggcctctccttgcctccaggggctccgtgtctgttagatgtgtaaacagatccatcaccgagcatactgatgagtcctggcctagggacaagcatggccacaaatgaggtgtctggaaagggctcacaggggaaggagccactgaggagggcaagaagaaggcttggccaggaagatggggtagggctcattcctggcaggtccagcctatgtgaagcccagagacccagtgaggggcttgcccatcctttggccttgctggagccaagggtgtgaaatgggagctgagagtaggatgttagtagcagtggcttcctgggaagggcccccacatgggagcctctggcctggttgcaaatggcaggaccaaggaggtggttgttgagttagttggaaggcactgtcctgccaggatttcccacgtcctcgtttgagcattgagatgctgaacgttaaactgtccttcattctccagctcagttcttttattcatccaacaaacacagagtagctactgtgtaccagatactattctaggcactgggcatacagtggggcccccagctcgtcctggcctcccagcccagtgggtgtggagcagtgagcaggaggacctcgagtgtcacacttgctccccttggtacacattggcacatggcgttgccaggtcagatattctcttcttggggttcaccagcatcccccttggatgtgccctccgtggtatgtggccgcttccatctcagtctcaggccgatggaagatattttcaaaattaacttagcttttggaattggttcctccccatgttggtgctcaaagactccccagtgagacagctgcctccttgagctctgtgtgcaaaacttggggagcaagacttgcttgagccagtgtttggcctggcccaagctgttgggcctgcagagcctcacctgcccctgtggctcagggcttggtgtctgaagtggatggaggcagctgaactggtttctccagggccccatccagcctgcgtttcccaggctgctgtcccagctctgctccacccaagtctactctgccgtcccctgatactgacactgagtgtgtttctgaacactgcagggggtgaggaataaggtgggcatattgtagcttcaggaggtgatgcttgtgtctgaaatataaggaccacaattgccatgcaggtgtaaatatctccagtgattacacatttccctgcaccgaccgagtggctgcacccagtctggggctctgtctctcctggtcagtctgttcttcctgaagccaggaacacaggagggcaagtttctctccatccctagtcccaccctggagccagcacacagtaggctgcaggaagtgctcgtggaacaaacacaggcagcaatggaagaatccccaccactgggattttcagcagctgatcgtagaagggagctggactgcaaggccgtctccgaccgcctccctctcactgcccagctggctagggtgatgagtatggacgaggatggaggcaggctgtggctgctgtctcatccaagtagccttttcctgacaccccaaggctgggcagagggactgagttggtcccagggcggagcctgctgggaaggaaggctgttagccgctgccttgcaggtgccatagcaatgccaggcagcccacctgctccttggattagatgggggatcagagccgtgagggaagcaggcctgtacccaggaacaggctcatcccgcccctgccctggcccggtactgatggacctggagccatagagtgcctctccccgcaacacacatgcacatgcatgcacgtgtgcatgcacacacatacaccacatacacaccacacacacacaccccacacatacacacataacacacacaccacacacacacacacacacacacagccaagcatcgattctgggccctgttctgtatgcacaccaattttcctgaacagactctccttgtcttctagcacccgggaggattcagaagtgtcctggctgccctgtggtgatggggatggcagggacctgcatttttgtgagttcctgcacgtgatgaaggggaaattgcaactgcccaggcagcctgtgcaaaaaggcagtgggtttcaagtgtggtcataaaccaaaagcctcaggttcacacgtcatgggggaggctgaggggaacacacagcctctgagctgtgctctcgggctgattctgagctcgtgctggtggggacacccccagacttccagcagcactggatgtccctggatcctcacatgactcatgaggaggggctggcatgttcctgaacttcaggtgttgactctgcagtgaggcgatttggatcagagagactgtgcaggtgcccaggacacatagcaggtgaggtgcacagccaggcttggctagcaattgggctcttaatgatgctcacactggacgtgtcagagtgcctgctcctgggcgtgcacagccttgtgggaggggccgacaggtggacagggaattatagggtggggggacagggagctgtgctggctgttgtggggacaccacagggaaggccccctgactctcatgttccagcattgccacttcctgtcatcctctcggcaaccctatatgaatgggaataggagtaagtgctctgtaaagactcctctggctgccaggcacgtgctaagctctttgcccacaccactcaggcattcttcatgatggccctaaaagtagagactcttgtgtccccattttacagagtaggaaagtgaaccctaaagaggctgcctgctgttctgcggccacgcagctgggcagtggcggcatcgggatttgaacccagaaactctggctgtacggtcttagccttggctcactctcacctccaagaaatagactctgagcagtgaggtgacgtgtcccaggtctcatgggggctgagggatctgggaagggtgatttgcccccacccctgcatctcccacacggtcccatccctgaacccttgcacacacggcccctctgcctccatgcccttcccctcactttgtcaaaccccagcagctgttcagcaggcacaccacagccgcctctggaggaggcattggctggtctcctgtgtcaatgtatcgacactgcctggcgaggtctggctctccggcctccctgtgtatccacaggtctccccagctctgcaatccctttagggcaggctcaggtcagtcatgctatggtcacccctcagtgccccgacatggctcagcagtgtccggtggcttgaatggaggaccacaaactgtcctctctctgcagaggggccccaggtcatatctgctgacttagcaccgcctataagatttaaagaccactctgttttcacctggaggaaccgtcagctcacgcaggatggcaggtggttttccagggctccacagtgaggtctccagttcatcttttgatgagggtgatgatgggacagtggccctgaaagcacatggctctagagtgcttaggagcctggtgacctgctgctgtgacttcctctccagagctggagctccaggggctgaggttccccttccctgggcagccccttcctcctaatccggctttccctgtgaggaagagcctcctggccttaagccacatccttagcagcttctgccgtggcccttcctccagccggggaccctggcagaatgcatgggcaggaggaggtgccttggccccaggacagccaggtgggcagtgaatggacgaaatcagtctgtgcaggagggaaacactaacactaaccctaaccctaaccctaaccctaacacattctcaaaacatttgattctaacttaaaacacacaagggcactcctttccatggccctactacatgatctggcctgacctgcctttctggcctctgctcataccactacctccctgatctttcagttcctcaaacacaccaagctggttcctaccctctagctccctcccactgactcctcctcaccctgagatcctcctcagcttatcaaaaaaagattcccccattttctcacttcaaccctttgttcatttcctccaaagtactgtattacagttaatgattattttttgggtctgtttacctgtttgggcttcgcctccccagtggtatgtaagatctttaagggcagggtgcatgtgtgtttcttgctttccaaaatatccctagtgggggccgggtgtggtggctaatgcctataatcccagcactttgggaggctgaggtgggtggatcacttgaggtcaggggttcgagaccagcctggccaatgtggtgaaacctcgtctctactaaaaacacaaaaattagctgggcgtggtggtgtgcacctgtagtcccagctacttgggaggctgaggcaggagaactgcttgaatccgggaggcagaggttgcagtgagccgagattgcaccactgcactccagcctgggtgacagagtgagactgtcttgaaacaaacaaacaaaaaactaaaacaagaaaacccccaaaatatctccagtgggtagcacagagcctgacacacagtaggtctgcatgaatatttgttgaaggaatgaaacagtgcctggatgagtgtattgcagccccttccaggtagggcacagccatcggggagccagcgtgacagatgtgagtgctgtggccctgggatgcgatgacaaccccttctcactggggtgtaccctggattggggtggtgcatgtagaatatatggcatgctgctggcacttggcaggtgctctgtcaaggttagttgttgttacttaaaaactctaatcaagtaagcatgacatatactgaggtcctactgtgttactgttatgggctgaattgtgtctgctccaaaactcacatgttgaagtcctaacccccagcacctcagaatgtgactgtgttttgagacagggttttcacagaggtacctaaggttaaactggatcattaaggtgggccctgatccaatatgactgtgtccttataagaagaggagattaggacagagacacacacagaaggacaactgtgtgaagacacagggagaagacagccatctgcaagccaggagagaggcttcagaagaaaccatcctgctgacaccttgatcttggacttccagctccagaactgtgaggaaatagatttcttttgtttaagtggcacagtctggggcactttgttatggtggcactagaaatgaatacagttaccaaatttaatttttcaagctctctctagcagcaaagtatttcccccatataaaaaaatgaggacgctgaggctcagagaggtgaactcgtttgccggagccacagggcttgctggcgggcgttggcactgatagttgaaccctggtccactagtgtgcctccagagatggtgcaggccacgccgtctgccttggtctcacgcaagtggcggctaagcccagaggcacttgtctccatttcagtgccctgctattggctggttagaaagtcaatattttatggagcctcatcaacctcccaagtcttgggtctcaattcccccttttggtgtgagcctctgaagactggttgagcatttgtgagaaacaaagttttgactactcaagactctagttaaaaaaaaaaaaaaattgcaccaatgtgccatccggctgcagcgtctcaggggcatgattcggtttttgcaacagggagtagctcttcttgaaacattatgggatttctttcctaagttgacttcacttacattctgctcagcacatgcaaagcatttggttatcgggaggcagagtaagatgacaaccctattattgaaggtaagcagacctggcctgttcacaaagccctttttacatcttgtttcattctgttaccaacagggtaactgtgaagtgggtgggcaggaactgtgacttcttttcaagtctcagaagatgtcagtcccccagggaggctggcctccgagtgccagtgcaggtggctccccctcggccgtcctatgccacgtgccctctcctcgtcattctcagccccctcgcgtctggagtcacctcgctggtttatcgtcttgtggtccatctgcccgtgagcattccagccaggtctgtgggcttcgttttcacggccgccattttggctgcccgcagagtgcctggtgcaaagcggatgacactcagtatctagagcggcacttccgggtaatccacaaacatttatcaaacacttaggtgtcacttggggtgctcggaattgcaaacatgaggacaaaccctcactccatttggggcctgcagattcagcaagagtggaaaaaccagctttttaaaatggcagcctgggaatcccagaattaggaagcacaagcttgtctttggcaggccaggccagctgctgacccgcacggccttcaggggccagccagtgcgaccatgattgaatgcacagccctgactcctttgtggcattggctcctgggtccaggtgggagcatctgattggcccccactggtcatgtgctaaccaggaagtggtgtgtgatgggctgagcctagggctcatacctgcatgtgttttgggggaaacagagagaaaggtgtggcctctccacttctgtggacaccccaaataggaagaagggtgcacacaaagcaccgcttcctccacgaagccctcctcaggcacatcgaggagaaccagctgcccaagctcttggctgctgcccttctgaggcctgcctggtaggtggcgtaggaggaggcttggtaagcagatgatgggacatcatctaccacatcccatcagtaacatccaacaagtgtgtgtgccagcacagacctgggcactggcatgtcccagccgtcattccatgtcaaatacttatctgtgccgtcatcgtcgaggtctggtttccaactctatggctccattgtctcaggtttaaccagtcctcttattgggcatcagcttttccatttttgctctgtgagaggttgttgagttagttggaggaccaagcccataatttcctggatgacagttcgtgtcccgcagatagagcttaaccaacagtggttccctttccagacctaagcaggtgggagtcagagatgagcatagagaaagggtgaaggggtgcctctggattcccaaggcagggacatagctgggggagctgcctccggactcgcaaggcgggggtgtggcagacactttgcagaaaaagtacatctgtccttggggcgagaggccgcacatgcagccccaagtccctggacctgtgagatcctggctgcctgctgttgatagaacgggacaggtggggggatgcttttgattcatgtacggtgaatgatcttattctcacctttatcactggtggcacattctgatggaaggagacattggcagtgggactgggcaagaaacaggtcatttgtcatggctgtgtgccagcctcctgtcgttgcacagcctgtccttgggaagtcttgctcacctgtcatgtggagtagttgtatgtgtgtgagtgtgtgtgtgactgagagagacatacacatatgcacacacacgcagaagtgtctgtacctctgagaatatagttctccagaggatgcagaaaaattggggattgcagactgtcgtgcgtttaaatgtgtccttctctcaagaataatgtccacttggaacctcagaatgtgaccttatttggaaatagggttcttgtgtatgtcactagttaagttgaggtcatattggattcgagtgggcctcaagtccaatggctggtgtccttctaaggagaggacacacagagacacagggagaggaaggccgtgtgatgacagaggcagagattggggtgatttatctacacaccagaagccaggagggagtcagggagcggctcctccctcagagcttccagagggagccaggcctgcccacgctgtgatgtcacattcctggcctccagaactaggagaggataaagttcgattgttttccagctgtctgtgtgtagtaatatgttacagcagccacaggacactgatacccagacacatcccaggccctaacatcccagaagaggagtccctgatactcctcttttgcaaaatgaggtgtgggctcagagcaagttcgattttctgtgaagggcctgatagtagatattttagactttgcaggctgtgtaaatctctgttgcatatttttcttttcttttctcttcttttcttttcttttctttctttttttagacagagtctcactctgtcgcctaggagtgcagtggcagtggcaccatctcagctcactgcaaccactgcctcccgggttcaagcgattctcctgccttagcctcccgagtagctgggattacaggcacatgccaccacgcttgcctaatttttgtgtgtgtgtgtttttagtagagatggggtttcaccacgttggccaggctggtctcgaactccctacctcaggtgatccgcctgcctcagcctcccaaagtgctaggatctgttgcacatttttctttgtttgttttgttgtcatttgtttgttgtttgtttcacaaccctttgaaaatgcagaaaacattcttagctcttgggctgggtgtcactgagtttgccctaggggaaccctcaccgcatgcgaagctgagtgggcacggtttcctccgctgtcaacgggaatattgatggtttccctgagtgaggacccaggggaagcactgggctggtgcccgcctcctgtctctgtgcccaggcctactgtctgcttcacactttactccccagaaccaggcatggcacttagtacagggcaggccgagctcgggggagatttgcttgaagaatgaggaacgtttctggaagcctctctgaccacccagcaggcaggggcaggtcttcctctgagacccaagtcctctatgcttccctccatcctagcagtgacttccatgacacaccattgtccctttgggagcacaggactctggctgagtcatctctgaagtgaggtgaacccagcacagggactgatggcttgggaagagcagctcagcctatgtggggggtgttgagggtgcccaggcccaggggcgatttgatcccaccctaccctagcgcagccagaatgctggggccgaggttcacctgggagatgcaggagtttagagaagaggaggaggatgtttaagcgtgcgagctggtgataggagcgtggagttggagctagacccagacttgaattctggctttgctgcgtggcagcagctgtgtggcccttgatgagtgtcttaaccttgctgggctttggcttcctctccaggagggcatagcaagtcctgcctgttagggccatcctgaggatggggtgaccagagcatgagcagccgggtgcccagggcacctgggcaacagccctgtgggtaggtgggtctgaatggttgagccccacgttttagtcaagaccactctccttgcagagcagttcatgtccacttgcccacatccttcctgtcccagcaccgtctgtaggttgctatgatcaccccattttacagatgaggaagcacatgcccaaagaagcaagtgacttcccaaggcggcaaggcaaggtgaggggcagaatgaggaccccaacccagttctccccaccccctccttcccttccagagcccctgcctgctggaggcattgtcccagcggcagagggtcaggccacatctgccagagctaaggggagtctccgtcatctcaaatcattgggtcaaatgacagaaatctgctggcatgctgccttttgaggctcggcgacttctctgaggctttcctgatggctgctctgggtgtctggaatgagctgtggacgagggccgtcctctgagtgaccatgccccccacgcagccgaggttcccctctgcctggctcagtgcgggttctgagaaagcctgcctgccctcactggaggcacctctgactcactgctgcaaggatgtggccatggactggggtccccgggggcccccgaaagcctcagcaggagaacatgtttatccagaagcctgggcaacacccagagggtcctggcatgggtgtgccaggcagactgggcatggaaacatgccccgtgatgtctttgggatcctgggagcctgaggcttgttctcagtgtgtagggcaggggctgggtgccacccaaggggggcaaactgaggtcacagagagggaggcagtacccccagaaaaggtgattcttctcattggaagattcccaaggcccaaggcctgggtcagcagacagtggaacaagggtaatggtcatgtccttaattcacgtaaccccgaggtgggtccctgtgaaaatcccgtggatggggcctggtgatggctacacttatattctcagaagcagcattaccacctggacccttcccttttctcttactccctcctcctcgtggggtctgcaagggggtctggtaaatggcatccccctccccgcccaggggctggacccgtggagcataagccaaaaggggcggcaaagagagacacggaacctcactgccactgcccagctgccggtgctgcaggacctggcttaggttcaggggcccggtgggcttccccagtgctggaccccagcaggcaggtgtggagggagcagggggtagtggagcagagtacagtggcccctggctgttctcccccaggaagagaggagaggcggggctggactctttcccatctccctccccaatactccagcgtcagtcagtctccccccgacagggggaagggagagaggccaggagccttcccctggggcacacacagagagcccagcaggcttggaagaagtgtctccctcctcctccagccctgccagatggtcccagccctcctgcagggttcccgaagtggaaggatctggactcagagcccagtgctgctgcttggcatctgtggtcccgggctggagggcgtgttggcagctcatgcctggccagcgcatcggggtggctgcagcagaggatgggggaggggagcagggacaagaggagcctgcaggctgctgggtgcagccagggcttctgcttcaccccgggggtcacagctgctttctgttgggaaagctcccactggaggtatggggtaggcagtgctggagaccctggtggcaagtgctgagctggggcacaggtgaggatcaggaggagctgggagggacatccgcaggttgtgctgatggactgcctctgagtattttcacgggatggtggaaagaggctttgagtcagacctgggttcgaatcctggctcctactcttggtagcagtgtgactttatgtgacctttctgaacctcagttttctctgttgtaaaatggggaaaaccacacatcgtgggggcngttgtgtgcacctaaggaagccaccccagggctgggcacacgggtagccgattgtcatatccttgttatggaaggttgttatccttgttaggctggtgggcaggggtggtagaggagtaggttgtttttggaaaatggtggacttagtcttagacatgttgtgtttaaattgctcatgagaaatccaaatagaaagacccagggtgacagcagggactgctaggaacttgggataaaaaattggtcctggggtgctggtcactgatcctgtggataaaggagttggggtgctctgtttgggtccagctggcagagttggaccagttgagagaaacttctaggaggaaggtttagaagaagaatgctttcaccatcagagctatcttaatagctgtgtaatgtggtagtgagctccccatcgtaggaggtatgtgagtcatggctggaggactacttggaggtctgttgtagaaggattcagacaccagagagggttagactggatgaccttcagttcccctcctgggtgaccacagctcttcaaagcccctcactgctgcccgcttctgcaggattcagggttgaatgtcagaagtctgctggaatgctgtcgattgaggcacggtgacttggccaagcctttcctggtggctgctctggtatctggaatgagctatgggtgagggccgtcctctgagtgaccgtgcccccctcacagctgaggttcccctcggcctggatcagcgtgggttctgagaaagcctgcttgccctgactggaggcacctggaggtggttttcaaagtgtcactgtcaccgtgacccagacatgcttgagccccaggctgggaaagctttttttctttgctttaaaaaagcagaaacagtgaagcaagcataaatttgcctttgaaataagaggctgaatgtctgccaaatgcaaatggattgaaatggccggtggtctgaccttgggatgttccattggtagtttcagaggggctctgagcccccaggccggctgtaacagtggacatgctggcgctggccagtttgatccctttgaaagccccccgatattcacctccaagagagaaggccctggcaggtcctccagcatgcggacgaggcgcatgcactgtgaggcagcggctctgtgggaggacggggccttttgtccactgtcctggcattccatggccctgtgtccccagatatccgcacctgttcctgacaccatcccttcctgtcgtgtgagcgagatgctcttctgtggaacctgggttcctatggtcactgggagggtgaccctgagttcacagagaaaggggagcaggctgttcaggaaaactaagcacatgtggtgagcatccacaggcggctagggatggaaggcttttcatccgggctttcagatgaggcccctgaagctcagaggggttaggcaattggcctgaggttgcacagccaggaaggagtgatggcgtagtgattcccattgggctctcccaccccctgttcctccctccatccagcccagggcctcgtggatacattaggagcccaacaagagttcccctcccttctcccttgcttctctgagcctcggtttccccatccatataccaggaggaaccccctgcccatctcntagggctctttgacatggtgaaatgagatccagatactggctgtctgtgtcaactgtaaagtgctgtcctgagtgaccagttgttgttggtgcctgtagccatccttgaggctaaaagagactgctgcttgcctgatgctcctcccgccagcctcctgctttctgcgtgacttccctcgccccagcaatgaaccctggagccacctgcaaatgggaaagctcttttgttagtttccacagaatgcccaccgtgcacatgatcagagagtttgcgagcctagctcaggtcagtggaactggttcttttcgtacatgtaggaagtaatgcagggtgctgccgaataaatccactgcctgcctggcctctgatgaacttaccagctcacgccagttctagctccagccaggaggaggaaaaaggcatactaggccgtattttgtttctttcaatgtttatttacctgtacacacacacacacacacacacacacacacacacacacacatacacaggcaaaacttatgttactgtcccatgcataattctctagatttgggccacatagatctggttttggccattgactggcaacatggcagtagccaactgcagagccaccagggagcagcctcattctgattgagtataacgaagacgctgatgggaaagagagacacgggtgagcaggggctgtggggcatcatgagagttgggagggtccccatcgtggagctggccagccctgagtgggagccccggcacagtcctctgctagctgtggcctttggccagtctccacttctcagagtctcagtgaccccagctgccaccaccaggattgttggggtgtccaagaatgaaaataggggtcatccctggccctcaggggatatgtgggctttcccctcatcatgaatggaacaggtgagtgctgtggtgcaggctgagaggcggcagggtgttctctcacagcagggtcgggtcttgctctgataactgcaccaggacaaacatccccagcctctgggatgcacacccaccagcatctgcccggcaggtttaattaccggctgagatgggttgtgttgggctcactaagtatgctccataagtgatgaaattgggcaagttctcacctctggaggctctgaccccgagtagctttgaccctgagtagctgaaggaacaggcagttcagcctctgcttatgggctgtgtgtgcacacatgcacgagtatgagagtgtgtcagtgtgtatcgtgtgaatatgtgtgcaggtaagtgtccatgcatatgtgtaagtacatgacaatgtgtgtgcgagtacatgactgtgtgtttgtatgtacaactgtgtgtgcacatgtatgtccgagcacattggcgtgtgtgtgaatgtgagtgtgagagcgtgtgatttcatgactgtacatgttcactctgtttattctcgggggaaatagcaggcaatctgtaagaaaatgactgtgtaagtttcatgggtggaggcttatgatactgttagagtctatcttagtctgttggtgctgccataacaaagataccatagactgggtgattcataaacaacagaaacatttctaacagttctagagctgggaagtccaagatgaaggtgccagcagtttcggtgtctgttgaaggccctatgcctggttcacaggtggcgacttcctgttgtgtcctcacatgggggaaggggcaagggagcttttgtaggcccttcttctaagggcactaatcccattcatgggggctccaccctcatgacctcatgacctcccaaaggccccacctcctaacaccattatcttggggttagggtttcaacatatgaatttgaggggacacagccattccatctgtgaccgtcctcaatcagccttctccctgcagggctgcaaatgagggtgctgccggattcataagagaagatacattgtaacatccacagcacacaggcactaggttgggagccttttccagcactcagatcagggttcaaaccctaactgtgcccttcaggagctgtgtgacttgggcattccttcattttttattcaagcacacagctggcatctacatgcctgctgtgtgtcagggagatagaaatagaggggtgagcccctgtgatattaggcaggcagctttcccttccagggtctcatttttctcactggtgaaatgagggcaggggctcctcccctggggttactttgaggggtcagtaaggtgaacagttacagtctcttgcacagcccctgaacgttgtttagaagtcaatactctgttccatcccatcagcttcctcttcccttgttttgactggcaaatagtcatcaccttctgaagtcattaagagaaggtctttttaaactattatgatgccatcaaaactgacttttgatccaaaatatcttttaaacaagctgaccattcagtgaggatttgccaggctcctttgtgatccatccttttggggccagggtctgtggggcccagagctggacatcatggaactagctctgaggagctcatgttcaggtgggacacagtctcctggaatcacagttctaagcactttccaactattaactcatttaatcgttaattactaccatttcccagatgatgcaccagggatgttaagtaaccaaggaatatggcagggccactgggatagtgggctgttagaaaagtcagggtggaggccgggcncggtggctcatgcctataatcccagcactttgggaggccgaggtgggcggatcacgaggtcaggagattgagaccatcctggctaacacagtgaaaccccgtctctactaaaaatacaaaaaattagccaggtgtggtggcgggcgcctgtagtcccagctactcaggaggctgaggcaggagaatggcgtgagcccaggaggcggagcttgcagtgagctgagattgcgccactgcactccagcctgggccatagagcaagactccctctcaaaaaaaaaaaaaaaagaaatgaaaagtcagggtggagtacagacacaaattagagaacttaagaggcaccaacaagagtatgggcagtatagggagaaatggaattagcacggagcaggggcttcctagggaggaatgatgggaggagacacctccttcctgctgctcctgctgtgtgccagcctgggctgggagcgtcacgccatcagctccagtattcattccagccctgtgaggctggcagcagggtcctcctagtggggctgaaaggtgcaatggtgaggaggaggattgcaggggctggagtcctggccccaccacttcctggctgtgtgactgtgggcacgttactcaccctctctggccctccatttattcatcattaaaatgggattaggggccaggtactggtggctcacacctgtaatccaagcnctttgggaggctgaggtgggtagatcacttgagctcaggagttcaagaccagcctgggcaacatggtgaaactccatctctacaaaaaatacacaaattagccaggtgtggtggcatgcgcctgtagtcccagctacttggtggggctgaggtgggaggattgcttgaacctgggagggttgaagctgcagtgagcagagatcgcaccaccgcacttcatcctgggtgacagagtgagactctgtctaataaataaataaataaataaataaataaataaataaataaaacagggggaatagtagtccctactccatagggctgttgtgaagatctagtgagatcaatgttcatgaaggactttggcgctgcacacgtttctgagaaagggtgacctggccaggctcactggggtggtaagtggcagggatgggcttctccacgtctcccctgtgatattaggcaggtgtatgcagttgcttcacgtccagccacaccccctcacacacctgcagaccagccagcatggcacaccagacacatttctgctgcacctttccccacacagtacctaccacctggcgtgcctctctgcccatccccatctttttgatattttaccttccacctaaatccgaactgaaatcccagacctcactagctggtgggtgcatggggtctctgctctgccacttctggagctgctcctagtgcatgccccagttactcactcgtggccaccaccccccgagtcaggggtcgagagtcccctttgtccatgatgggtgacgagggcatacccggaacagagggctaattaaatcacacgctgaggtttgtagaacatgggacgcttcattaggctctgtgtgaacaagtgcgcccagcctgctcccactactccagcagactcgagcctggtataatcacagctcacactctgcttcatcagctcgctgaggctgtggggagggctcacacctttgggctctaaagagagtgggcctgaggtgtgggatcctgcttctgagcaggggtgtctgccccgagccagcttgcccagtggtgtggagctggagccccctctgcttaagtaaaggcctctgagggacacatgtcatgcagatgatgacaccagactggctcctcactttaaactctacttcctgagagcagaaggcagggaaggcctcagttggaggtgaaaatggaactttcatccaccagatgttagataaaagataggaagggtgttccaggcagaggggaaagtctgggcaaaggcttggaggataaaagggcaccgcatgtttggggagcggggcatgttccctggggccagagacctgagttctcctcttggctctatccccagctccacccatgacttggggtgagtcctgaaccccgaccaaccttagtgtctccagggctgggatatgggggggtggcagcctctgatctgtggtcacagggagtctcagctgtggtgctcctgtgcctccctcttgctactctctggccagtgacgatggcactttactctcctgcagagacctgtccccagtgcctgcctcctccagtcctcttcctcatgggctggctgctctgggcacatctcagggcagccccagaaggtgggctgagctgttatttggggcgtggggttctcggttcctccgctccatgctggtgggcctggtgcctggcaccactggcccgtcttccaggaagctcactgacaccatcctggatgggccttgttttctctctggcctgggagtcagctgcactctcgcaggtcctaggccaaggtgacctgcttgtttcattcttgtctttctcctttgctttatggtatatttttaagcataagatatatttatttgtaaaataaaagttcattataaaatgatgccttaattaccttcctgcaatataacaattatttggttgtatagttcctgttctccgtattttgtgtaataaaatggacccaatgggtgctgcacaatgctgggtgccacagggaacacacactgctgttttctcctggatttttaaaattttaattccagcatggtccttttatatgtgctttcactctagctgtttgcctacatctccacagttttgattgtccagggtggtgaataaaatgcaacacttggcatcttttaatgtttaaaaaaatcaacaagtattttatttaaaataaaatgtgaatatctgtaatcctaaaaaaaaaaaaaaaaaagccacaggatccttgagaggccctctgagacagtgacacccctgaccagctgcagccccctgtccactccccaggctccattctctctacccactgcctctgcatcctccgcccttggagtttctcagggtgccccgcagatcacctgtggcccaagtgctcagggacttgtttaaaattcagattcccaggccccgcccaggcttgcaggcagccagggccaaggaatccaaatactaatcaagcccactgtaattgccctgcatctgaggtctgaaatcactcgcctgctctggattatctgtgctccagggtggcctcagtttacaccatcctacctccccattgaaaccttactgctcacgatggcccaggttcccacccatcctgggagagaaagatgattggctcagttctctgggaggcctggtgattggctgcctctaggagggtgtggctacctcttgatccaatcagtggagggcagcatggggatgtgatcatgcagtgaagaacctgtaccgggggtagttaggcgccagggaaggggaggaggagaaaccagttctactgttagtatcagctcatctaacatttggcccctggtgggggtttttgcatccttttgggggtctaggtctcttctgccattttgttggcactttaatttcttgttaacaaacccaagagcacatacaacttaatttttaaattcccaataatttccagcatcctctaagttagtgctgagcgatagaaatgtaacggaagctgtacgtgtagttttaaaacttctgatagccaggttatgcacgtaaaaagaaacaggtgaaattaattgtaataatttgtatatccaaaatagtataaactgtaatcaatctaaagtaattgagacattttacattctgtttttcatattaaatctttaaaattcggtgtgtgttttatgcttaccatacatcccagctcagactagccacatttcaagggttcattagccacccatggctggtggtccctgtactggatagcgcagcactaaatgatggctttactttgtatccttggggaagtatgtatttttgaatccattttcctcacttttgaatagtttaagaacttcccagtccatttgtctaagggtatttgacttctaaatgttgggtttcttggccgggctcagtggcttacacctgtaatcccagcctttgggaggccgaggtatgcggatcacttgaggtcaggagtttgagaccagcctggccaacatggtgaaaccccgtctctactaaaaatacaaaaaaaataattagccgggtgtggtggcgggtgcctgttgtcccagctacttgggaggctgaagctggagaatcgcttgaacccagcaggcgaggttgcagtgagctgagatcgtgccaccgcactccagcctgggcagcagagtgagactccatctgaaaaacaaacaaacaaaaaaaccagttgggtttctagcaataaatgctcataaaatctgctggagttttggaggattagtccattcatcctgacttgtgtcctgtttctgctgggtcattgttgacttcccccacttgcctcacacttgtaaggccatgtggcacaccatcaacattcatctctgattaaggagcttgtttcttgtagtggctgccataacatattatcacacgtttggttgtttaaacagcagaaacttacactcttcctgttctacagtccagaagtccaaaatcatggtgtccgtaggcctacgttccctctgtagcctgcaggggtggatccttccttctctagaggctgccagcatctcttggctttatggccacattactctaagttctgcttcggtttttacatcacctcctcctctttcccccatgtcttctcctctgtgtgtctcttataaagccacttgccactgcatttagggcttactcagataattcagaatgatcttctcatctcagaatccttaattatgtcttcaaagaccctttttccaaataaggtcacattcacaagttccagggattagagtgtggacataccttttgttttttattttattttatttaatgtatttatttatttttgagacagagtttttccctgtttccctggctggagtgcagtggtgcgatctcggctcactgcagcctccgcctcccaggttcaatggattctcctgcctcagcctcccgagtagctgagattacaggtgcgtgccaccatacccagctattttttttttggctttttctttagtagagacagggttttcatccaacagatgttagataaaatgttgcccaggctggtctcgaactcctgggctcaagcgatctacccgcattgggctcccaaagtgctgggattacaggcgtgagccctgcaccctgccaagagtgtgtacatatcttttgtggggcaccattcaacccattgcagtgaggtgtcagtgttttgacaggaagaagcagcaaagaagacaaatcagcactttatttttctaagctttgaaattcttaaaatgatagtgggttaagaaagaccaacttccccatggaaggtgctggtccacactggctgggtttagatccctttggctgaggtttagatccctttggaggggagtcaatttgaagaagaagcacatcagacacattcaggaaggcaaacccaaagacagcagaaaggcaagaatgtctcaaattgttcaaaaataaatacgttagttcttgtaacgtgctccgaatagggtgaggctcaacatgagcacttaataaatgctcagtttaattgtggtaattgtaattctcattggtatatatttatgtcctgatcctatacctctgttattaatgagactgataataggtaagtgaagggaaaaagagacccccctgctgccaggaaacaaaacaaagtatcgagccaaatgccaaatccataagtcaccaagatgggttttgatgacaaaacctttctttctggactccccctgcccctccgcgaagccgttccccatggccacgcttgtccgtggctggctttataggcccatccttcttttatatgatcatcaagatggcactaatttgggtttgcaaatgaggttttcgtgatgttgtcatgtcagcatctggattcaagaggggctgcttcccagaccctgaccctcccctgtggcctgggagcaggcccagcccctccctccatctaatcactccccaggaacctgggggatttgctaatgggcccaggaagccataaagagtaatgaaagtgctgtgtgactgctctgcacggggttgcccttggggtcccttcctcataattaaattcggaatcctccttctgcctttgagctggaagagagggttttatcctccctgtggtctgttcatcccttcttccccccattcatttgttcagcacttattatccagttatttgtttatcaagccacagttgaatgcctgctgagagccggtttagggcactgagcttcctgttctcaacaggcagaggtgggagcagccaggctgctgtgggaagagcagagtggctatggagccgccctgagccagctctgctgtgcgggagttctgcgacctggggaacccgctcgcttggcttcctcatctgtaaaacggggagactaatataatagcagctaccttccaggggggctgtgagaattaaaggggacgatggatataaactcccacacactgtcgggcacagggcgttaatagcagccctcagtctcatgtcccacttattttgtgcccgacacacttctgagcatcttatgtctattcactcatttaatttgcccagtaactctaggaaggaggtcacgtccatggttcttattttgctgatgaggcagttgaggtaggcagaggtgcggtgccttgctcaagatggcacagtggaaggacacggggcagggcggacctgaccgcagttggccctcccttgcctgcctcttagcacgcatgcagccccctcgcctgctctgcccgttgtgtctcagaatctccactgccatttgcgggtgccctgaatccacctcgcttttccgtgcctccctgcttttgtgcttggtgcagacccctgcttcctcatgggcacctgcctccttagaaccccccttggaatctgttaactacagcttcctgggcccacaccagcatccctctgcaactcagaatttccagggcccataatttcctacaggcccctcgggtgattgctttctcagtaggcaacttagggccgtccctccaaacctttagagtgagaagcgtgctacctgggtgcttcataactgatgttgcaaatgttcaaccaagtgtaggaagtgaacacactttgtatctgcaaagtgctgtgcatacaagcgaggtcaggagcagctggcacccgtccccaagggtttccatgcctctggcgagatagggtaggagtatctgcaagtagctgccttcttggccatggaagaacaagttcaacagggcgcctctgtgtgcctgccccttgctgtgtacaaggaccttgcacttcagggacatcaaggcaccttcagccgggctttggggaacttgtgtatgggagggggggacctctaaggacctttcctgtcccaaggagctccccagaagcctgttgcagtgcccagcaccccagcctcatggttaaggtaggcagtccccaggagtccccttcctgggacatcaactctcctggtgggaacaaatgctgagcacctcagcatgagatgggtacagtcagggactccacaggcaggtcatgtcacagacaaggtcacacagctaggtggtggcaggttggagagagagggcagtcccctgactcccatcccagtgctcttttctcccatgacagtgacgggtgaaatggcacagtgtatccctgtcaggcaggaggggatttgttgtacttttttttttttttttctgaggcggagtttcactctgtcacccaggctggagtgcagcggcgcaatctctgcacactgcaacctccgcctcccaggttcaagcgattctcctgcctcagcctcctgagtagctgggattacaggcacccaccaccacgcccagctaacttttgtatttttagtagaggcggggtttcaccatgttggccaggctggtcttgaactcctgacctcaaatgatccccctgcctcgtcctcccaggttgtatttttaatcctccacaactaaatactcatgtcagtggaaaggttccaagctctttaaagacagcctctgccagcgtgctcccaggggaggccagggaaggctttgggaagcctccgtgggggtggggacctcctgcaacccctggcgcacagcctcaccccgctgtaaacagagctgggattgaagtgcgatttcggtttctttttctttttttctaatcaaataaaaacacctagggggcaggaagcgaggaggaagaggccagggagtaattcttgttgccaaaccagtttctaaggggctccgctccgctcccagcatttctgtctctgaggctccgacctctgagatgatcaatcctcccatttcagccagatgagaattgctgtgggccctgccttttcttaatattttgcatgagagcgacagcccggccagcggcagtaatctcccacccacgtggggaggcacccactgtcctgccatgtgcataattgaagtcttcagactgctcagtggttctaattagcccaaagtggccccttctggcctcagtggaaattactgccctcctgccgcctggtcttactagctgatctttgaaggtgtgagtgagccgggagacgtgcagagcctagcactccctctgctgcattggtctttcttggcaggccttcatggctttgcagagggcgggcagttgaggcctctgtctgccgttgaggtcctgcctccctcctctctgccaccactctccatgggacaggtgctccagctgggcgcatgcatgcccctctcacctgaatgcctcccactagcagtgtctctgctctttctgacccttcaggttatgcagtgcctcctcctccaggaagtcatccctgatccccaggctgggccctcagaatccccatgtctccctccatcgcagccctggccatgccatgtggtgcttgtttacatccatgactgccccatgagactgggagcccctagggggcagaagccgctccatgtcccacctactcctggcctagcgcggtcccataggacatgtctacggaaatggagggcgtgagggtgggaggaaggaaggaaggcaagaaggaaagaaggaaggagaaggaaaaaggagaagagagaaaagagggagggtgggcaggtagcaccatgcccattttgcaggtgagaacactgaggagacataaccgggaagtggcagggccaagcacacccttggcagctcattatactttcccttcagcctagcaaagctactgttcgatttggggaaatggggccgcttgtatgaagagccgcaggcaaagtggaaagtgccgagggctggaagtcagatgggtgtgggtgcgcagccttcctgtgtacctgtgggcaccacttacctgctctgtgaccctgagcagggcacctttgccttcgaaacttgaagatcacagtcgtccaggtggattcaaggcgatgaacagcaacagcgcatgctcttcccaaacagtattcttcttatcctcactgaagccccagaaggtagttgatccttaccacatcattaacctcaatgcgcagatgaggatgccaaagcacagagaggttgagcaactggcccaaagtcacacagtgcgtgagtgacagagccaggatttgcgcctgggttcttggctctgatattagtgccattgactccactctgttgtgtgaggtatgaaggcatccagtacagtgtctggcatcggcaggtagcttccatggcagataccccccctctcccaccagattctgccctttgagcgacaggaagataaatagcgctgttctccagtgtcacttgtgactttcctctgtgagttcattgtcagggtgctcctgagctgagctgtggccaccactcattggtgtggtccctggagacacttggataaaattgtgatgacagagggctctcaggctcatgagatcacacagaggagggtggagctcaaagggctatctcagaccgggatgctccagaggttaggtcttggtgctgtgtttggcccccagctgctctttcaggccttggtcttgcccttgcccttggtctctggaaggctgaatagctgcatcttgttcatctggaaactctctgggatgagtgactttagcatctcggatgttgccatcgtagcccatgtgcataacaacttattgtgcaagcatgtacagtcatattgaaaataaatgtagaagtgaaggaggcagtccaaagacggttcctgacaatactcggaaacagcttcattaggtattggccagagcttctatttgtgggttgctttggcgaaactgcttccttaccattaaccatatccaggagatggaggggaaagtcttcacctgggttggtttttctcagaggggcaggcaagctgaataacgggagtactaacatttccaagaagattaatgattaaaatgttgaatacaatttgagacttgttttcatgttcatattttatagtccagaagttctttctataatgtcctcataaatattttatttggccttaggttaaatgtcagtttccatgtcctgccacgtgccacttgaccaacgtgtgtgggaacgtctctactttggacacactgtctatatgaatggaggcccttctaccccttgaaatgttcctcaactcccagctcagtttcccttccgcatccctttgactgcatgagcggaagcgaggcattacagagaggtttgaggagccagccagcaagctagcaacggcttcagttcatttctgagatgtttgcattcaggggtcccattggccaaccttaacttctcctttgagctctaggcatcttgtgatgaatggcataagccttagaacatgggagaggcttggatgggccaggagctccaaacactaaggaatgttgcttacatcaatagacgccttagttacttaggtcaatagataccttagttgcttagatcaatagatgccttagtagatcaaacacccaaatcacccaatgtacttttagggcaatggttgccaaactttttggtcttaggacccctttttacactcttaaaaattattgtggctcccaaagaggttttgtgtatgtgggctatatccttcaatatttgccaactttgaaattaaaactcagacagttaaaaagatttatatgaattcattgaaaaataaaaagtgtaaacccattacatgttaacgtaaataatattttttttggaaaataactgtgttttgttttaaaaagtgagaagagtggcgtgtgtatcaatctctttaatctctagcttaatagatgatggttggattctcacatcagctcctgcattcagtctgttgtgatacattgtttggttgaggtattgtcgaacatctatcctcaaacagatatgtagttggaaaagggagaaccccacaatcccctgaaaacgtcttggggtcccctggaggtacttagaccacagtcttagaactgctgatttaggtgatgggtgaaactcaagatgaagacaaaagagaaaacatttcaaccccaagagacttcaatgcattcacatgcattgactcaggatcattaaagctctaactgaacccaacatggagacagagcaggaaacacagccatggctggcttgacttcagcctcccatcaaagacccccatctttggctcaactgcccccagtggtttcaatcagccacagacttcccttgtccagctggactggacgttctgcatttgactgtctgtcggctgcccactgattttcaggctgctgaattacccattagctcacctgaacccctaatgccacatctaaacggtcctcactctgcctttctcagcatccgagaatgtcctataaaaggcttcttccatggaggtgggcagccttgggcctctcctagggtcagctcaggctgcccacgctggctatggcttcctgctggcccctggaagctgtgtacacctgggagagtgaggtgaatgttaatttattcagcatgcaattaatgaattctaccctgtgcctggcacgactcatttctcatgccaaagctagggtggatcagaccaccttggacaaatctggatgggtgaacttgtggattgatcaatgatcttagttgatgaaaaatgcaccaaatgactgtttcctttaatgcgattgcttgggtggtggactcgattacgttgaggggattaaattagcagcttgctgggtttcagatgagtgcaggtggctgctggcacttcctctcccctcttttctttctttcccttttactgcaaacgaagacttttcaaagctttactaagtacaataaatgccatccccttaaagtgtagacttccatattcttagaattggcaagctatgtatatgtttgtgaaatcacccccataattaagatttggagcatggccttcacccacaaaacgtttcctgttctctgtgtggtccattcctatctctgccctgcaccctgggtagccagtgatttgcctttggtcactgtaggatagtctgcactttcaagaatttcatatgaatagagtcatagatgcatgcattttttggtctggcttctttcactcagtgtgggtattttgaacttccccctggttgctgtgtgtgtcaatgatttgttcctttttctttgaggagaattctgttctgtgaatatgccatgatttatttattccttcccctgctgatgaatatttgagttttttctaattctgagctactgtgcagaaagctgctatgaacacgtacgtatgcacctttgtgagtttgtgagctttcgtgactcttggttacaaggatggttggttcgcatggtagacccgtgcttattttcacgctttaagaaactgccttgctgtttcccacggggctctatcgtttttcattcccccagcagtgtgtgaggggctgtttgctccacctcttcaccaactccatattattagtctttttaatgttcgccactctgatgggtgtacactggtatctcgttgtgatttgcatctgcgtcttgtgatgactgatgatgttaagccccttgccatgtgcttcctgaccattcttgtattttggtttgggaagtgtctgcccaaatcttttgcctattttaaatcaggttgcttgtgtttatattattgagctgtaagaggtctttatacatccgggctacaagaggtttactagatacatgtactacgaatattttctctcattccatgccttgccattttcttttcataacagtttctttcacatagcaaaggtttaagctttgataaagttcagtttgtccattttttgttgtttttcatgctttttgtgtcttgagaaatcgaagttgcaaggagtttcttctataatgtctcctagaaggctcatggttttagcttttatgtttaggtagatgatgcatttcaatgtcagtttgtcagatggtgtaagagttgatattcattttgcccccagtcatttattggaaagactgtgctttcctcgtggaatcacctgagtgcctttgatgacaatctgagttcaccacagacttgtgggtctagttctggattctctgtttagttgtattgatccacatgtctgtccttttaccaggaccacactgtctgtatcactgtatctttatatcagctggggtaaatcctccaactgtgttcctttatttatttatttattgcaaagtcattttggctattctaggtcttgttctaacaggaacatattttcgcttatttaatccttaggaccctctgaagcaggtgtgaatattaagcccattttatggataaggaaactgaggttcggagaggatgagtaactttcccaggtaactgaggaggaagtgagccttgcccctggtaggtctggcagctgcacttgcccttagcctctaggcaggctgcactgccctctctgtgggttccaaccattcctggtacacacccctagccaagaacttgtgaggttcttggaacctcatgcaagaggctcttgcatgaggcctcttttccaacttctgtttatcctgtcccaaagaggtcagtgagggacccccagggccgcctctgttccctcaccctgcaccttggccgggagacccccgcagcgcagggagcatccctccggatgtttcttcccaggcaacaatattctgtttatcaagccaggccccgaggctgaggcctccccctggcctggggtgatttttggttggtggtgagaagcagcaggggtgcctgggggcctggctgtcatcggagccagctccctgtcctggtctctgctgtgcgggatggcatgggtaggggctttgtgcaggatgagggtcctgccgactgggacgtgggctttggaagggccacgcttctcttacaggggtgcccttgtggctgtatgaggagtccaagctctgaggttgaacactgggattgaaggattcctgccagggtgcacttaggtggctcacttggcccagagtgagccagagctggctcgcatcagtgcgcgggagtcaatagttacatttttaggaattttttgagccagttgttaaactgtgggtagcatggaaatcagccctggcttgagtaggctcgcatcagtgcgcgggagtcaatagttacatttttaggaatgttttgagccagttgttaaactgtgggtagcatggaaatcagccctggcttgagtattcatgccacggacttggcaaatgccgcaaatcagggccttctctctaccccctcccagccccaacccgaagacctgattgttaactgcactggcaaacccctggctctgcctcctggagcctcaggttccttatctggaagcggggagaataatgcccacaccttaaggcatcatcaacacccttaaatgagatcatgggtatgaaatgcttgtgaatagttttgagtgtgatacgcaagtgtgaggggtgctcaccaccctggccattgccattgtcctcctcatggcagcagctggacaatgtgcccaggctgggagttggtgtctccaggaaaaactggaggaaaggagcccgataacccatgggcagcagctgggttagggagggctccagacctgcctatctccgtcagccctgctgaatgtgcctctgtttgctcatctttataatgggtgcactgatcccagctgagaggggtgtggtgggctttggggtacaggccacaaggtacaaggcatggcctgactgcagctccgtcactctcatggctcaggtgcatggctgtcccacagtggaaggaccaggggctggcctgatctgcacttcatggcatcaagagcacaggccctggagtccaccaggcacagagaagcagcctgctcctgggtggagtacaagttcaaccctcgggagttgtggtttccccatgtggaagctggcagcatgccacgcacctgccagggctgccgcgagggttcgcacacactgtgtgtaatgctcctgtgtgagttccccggtgacccaggtcccactggggtggccggcgtgtaaccaggacaggaacatctccaaagccactccttgtttgctgcctggtggctcaagcagaacatctgtgcctgcagggggcccataagccttaatgccctgcagagagggggcctgggggaggctgggactcacaccagctggggaacaaggcctgcagctggggggctaccaggatgctccttgccagtaaagagggtcctcaggagatcagccacagctcaatcaggagggtctgcccatttcacaggggaggaaagcgagcctctggaggctgagctgccagcagtcacggggccagcgcacagccagagctgaccactctgtatgactggcgggtctgcgcccttggcataccacagcagctctcacaccaacagaggctggacctgctgatcagcccccgctgtggggggtcccgcctctgcccacaccctaccccacaccttcacaggggtggactgtcaccctctagcagactgtcctccagagaccgagacacggagcctctccacaaggagggaggaaagggaggtggaaaccagccttaccctggacaccgggtggtccttgctgcatacccaaccctcattggtcagactggtctgggagggcctggacatgcccgcggggtccgtttctgaattcccgttgcctggcataagctctggcacttagtaggtcctcagttaatacttgtcagataaataaatggcctcctttggggaaatgaagaatgtccaggctggccacggggaaggggcagtgttaggagtggagagcgagagggtgagccaggcagatgggacctttattcaaagggcagtggggagccatggaaggttttagggagggccatcggatgaggttttcattttagaaaagtccttctggctgctgggtggatggggtcaagatgaagacaggaaatccagagaggaggcggctgcaggctcgcgggtgagaggtgaccatggccaggccagggcggaggcagggaggtgggcggaggggggctgctggctctgccaactgtgaccccagcacccagctcagggaattatggccagagacaaagcagcttagagcctgacccctgccctggagcagataggcctgggaagggggctgatggacacgctgatgtgatgggacctgagaggtgttcttgtcaccaggccagtttggcccccatcgcccgcctgccggctggggtctaaaaggctggctgtagccccagcagagaggacagggcctgtgggaccccaggtgctgggagcacagaagccagccaggccatctggcccggctgagactcaagtgataggtccgcatcagtgtcaggcctccatgccccccatcctgggatgggaacagcacatcacagtttgcagagccctgcggcaggggtgactcttattgccattttgcaaatgggaaaactgagacttagagtctctctgactcacataagcaggacaggagacagtcgaaccaggtcccccagctgcaatttaccacatctcaaacccttttcctctctctgtcattgattaatctttgcttcaatgtccacatagttctgtgatttgctttgggtacatagtagcaataattatagcaacaatttctagcagtttcttggtgccagacacttctgtctttgttgcctcgttgaatcttcaccatcaaccttgaatatgaaacccattttagagattaggaaactgaggctcagagactgtttataacttactgagaatcaggcagccccactgaggtgctttaccagtctcccctttaccagtggttctcaaaccttggcacaccacaggaaaacctgtcagaatgcagattctaggccccagcaattctggtcctgtccgtctgggcagagccttggcaagcttcgtctttaggagagaagcccaggtgactgggggtggggctttccggagcctgtgttgcaagaaggaaggcagggagcatgtgatcttctctcggtctccctcaccttgaccctgtcccaaggccagctgctggcctcaggctgcctggttgcctttgaaggtccatggggatccgatggagggttggtcgtgctgaagccaggctgggtgtcatcctggctctgagcacagtccagcctggctagagtcaaacccagcagcttgaccggatcctgctgaccacagacaaatccacaactaccccgtccacctcccccaggcccatctgcctctgtgccagccaggctggcagacactccatcgggcgggccccacctggcttggccagggagaaactggcagccacagcccctggcctccccgatccctgtcccttgtgcactgcccatgggaggggcagtggccttccacgctgggtctggctcctgcagtagcagagctgggtgcagacacgggttggaggccggggccatgggggcattcgtaggctcatgagtcccttctcactctcttctggggactgggtttctgctggaagagaaagggaccagccttaaaagacatcgtgattaaaatggaattattatttcagcctggaaaaggaatgatgtactgatctgtgtcacaacatggatgaaccttgaaaacattctgcgaagtggaagaagccaggcagaccccaaaggccacgtgctgctgtatgattctatttatgcaaaatatccagagtaggcaaaatccatagaaacaggaagtagatcagtgatttccgggggctggggagaggaggggatagggaatggctgccgacggatgtggggtttctttttggggtggtgggaatgttctgagtttctttttggggcggtgggaatgttctggcattagactgtggtgatggttctagaattctgtgaatctgttaaaatccaatcaagtgtacatttaaaagtggtgaatttcatgttatgtatattgtacctcaactttagaaatgctaaaatagaaacaacaaaatcatcatgatgcaacgcctttcctcatagacctggagcgaggcctgagagagaagccatataaggcagatgaggggtgggatggagagcttcctttcattcgctgcctgcccagtgagggtctgcttcctctggcacttacttaacaaacccgagagtcacccatgtgtgaggcatgggctgggcacagaagggtccacgcccacccctgaaagtgctcgtccatgctagaaagacagaaggatggggtggtcattgaacctaagtcagaccgtggcttcccagctcatgcagaataaaatgctcaggcctctcccggcctgctgggtgcaggaccccggcccagcccacctctctgagctctgagctcatgtcctgccccatccctctcacacgcacactggcctccttgctggcctgcaacagatcaaactcaccctacctcagggcctttgctcgtgctggtcacctgatatctgtgaggctccctctctcacatccttcaggtctctgctcaaatgcgtctcccccaggaagtcttccctggcctccccagctaaaacagcccctcccacccctcccatgccatttaccatactatagttttcatcttagcccttatatctccctccaagagctatttattgacatgaaaatttgcttattggctgtctctcccattcgagagtgggctttctctggggagtgtgcactgctgaatttccagtgtatgcaacagtgctgaggagagggtaggtgctcagttaatgctttttaattaaattttctattgtggtagaatatacatgacatagaatttaccatctgaaccatttttaaatgtacaatcaatgtcattaagtatattcacgttgctgtgcacctgtcaccaccatccatttctgggactttctcatcatcccagacagagactctgtacctgttaaacaagaactcgccattcattaatattcttttgaatgactaattgaatgactaagtgtatttcctgtgtgtcggtcacaagctaggtgatgagccagtaaacctggcgcctcggagggtgtgtgttcccgtatctgtctcggagatggacagatcgactcagagaggtgtgaagggctttgcccaaggtcacacagctggtcagggtgggattcaaacgcaggtctttccccctttccacacagtggcagttctgggggatcactgcatgcaaagggcatagccatggcacctccaggcctccagccccacccagggcttcccctgctgccgctcccccaagggtcactgtgtcggccacatgttcctggtgtcctcggctctggcaggctgaccaccagtcacaggcacgtcttgggaccacacttggaggtttcagcttcccatttcacctggtgctcttggggtggaggccgggatcatggaatcctttttccatggcctttaacacaaatcaatactgtgcccagtttaaatgtttatgtaactcatcatctgccggctctgctattacggtgtgcttttctttttataaaccaagctcaggggaggtgatattaagtccaggacttgcctggcgaggcccagcgcgtgtgcagaggattcaatgtggcaataagggcgagttccctgcacagcgcctgccatggctcgtcccagcctcctccctcatctgcttgtgtcctctacacggtagggaggagggagaggtccagagaggggaggtggcttgcccagggtcacagccacaatgaggtggaacagggattgtttccaaggtgtgtgtgggggtgggacccaggctcctgccagaggccagggcggcagagccctgacaggcagatcctgggttcctgggtgtccaggttacgtgggtgggcaggtggcagctggcacccaccccatacagacactctctgacaccccccaaaaatactcccaccagccccttcacattttcctccagccaagtgaccaaagcacagctcagggcccatttgccctggccacactgtccccactctccccacccaacctctgaccacaggcctgggagctgccccatctccaccaagttcaggctgcaggaggaaacgtctcaccaggcccgctggaaggtggggtggtgatgaaatgtgggggttcttcagcgagagtggaattcagtaataaggtgatcatcactgccgggcggtgcctggactcatgatgagccaggactaggtgagtgcagagggctggtgagcaggcctgatctgtgaccccggtggccctgcggctctcagacccctcacataaaatcccatcagggatattccgttccctggcccggaatgaggtcttcgtctttctgatcgcccttggagagcccaacggccttccgtcactcactcagtgctgatgtccagtcccggcaacagtctgactccgcccagacttggcgcttcccctcccatgcgcccgccgcccggcgtgctccctgctggagggtgacatggggaggggcctggctgcagcccttttcaggcagctcttgttggctgggaagggagcctggggatgggccgccttctacctgccggtggctgtgcccactctgagtgtgcttggtgggtgattacagctgctcacagctgtcaggagtccttcacagggcagtctggaagtttcctgtagagctgtctcctgcagagtccttacctagggagaccccgctgtatgtcctgtggccagtagcatcctgtggctttgctcaatcccccaggcacagtgtgtgcacccccagcctccctctgctgtgttctccacgcccctctgggcagccggcactcaggacctaaggtggtcctaagcagctctcccctgtcccctggaagtacgtggaggtaaaagctgcatccctggcagaatcagaccccaaactccagacccttttgctgtgagtctcaggctagggtctcccacctgcaggaatcattttgggccttttgagggtctctttcatagacgtgggtggggcgtctgctcagcacctgctttctccttcaccaactcctcagcctcccatctctgaccccataaacgctgagaggagaaccaggcctggggggcaagttcaaggactcaggaggcagaccgctggaatccaatctctcccctgcccttactagctgagcggctgttggcaagttgtcttctctgtgcttcagtttcttcatctgtgcaatgggggtgataacagcacacaagaacatcttggagttgttaagaagattcagtaataggtgtaaaatgctaaagctggggcctggctcccaggaagtgcccagaaggtgtactgtgataaccacagacgccacgtcaccccgctggacctggactccgtgaggagggggctccccatctccttatcccagtgtcagcccgctgctgggtaaattcgctcgccaagcccataccaggtgctaatggctgcaggagcctcttcctctccctgacatttcccttcggcagcaggcggacccctccttcctcgcccacctccatccttagcagccctgatacctctgacaatgctaattaaatgcaacaacctcatcagaaggctcgagctgggcttgcctaaggccccctctctgccctgagccctgatggatcattaatggtgtcagcagcacttatggtgatggacgaggggagcaagtcccccgacgtgctggggaggaagtccgtgacgagcatcttcatctcctgtctgcaggcctcggccagagtttccagggaacgtcctagtccacagaatcgtttgaatttcacatttaccctgggagaaccatggccaggcagggatgtcaattcccattgtatccatgaggcagtcagggctcagagagggcaagagatttgggtgagggcacacagctggtaagtagtggggctgggactggattaccacggctttgatgaaagctgcagggggcatctgtaagatccaagggcgatgtctcccccaggtggcacgtggagaagaaagccagcaagtgccaccggcttgggggtagcccagttctcgggggctggagcctgcacactctcatctcagctctgtgtcagtttcctagggaagctgtgacaaagtgctacaaactgcgtggcttaaaacaaccgaaatttgttctctcacagttctggaggccagaagtctgcaatcacgatgctggcagggctgggctccctctgacggctccagggaggactctgttccaggcctccccaccagcttctagtggtggctggcaatccttggcatttccgggcatttccaggcattgggaggcccggctcggccccccaaagtgctgggattacaggtgtgagccaccatgcccgtcctgcccttcattcttgatgcctcccttcagcctctgcctctgtctccccatggcctcctccctgtatctgtgtgtgctgtgtcttcatatggacttcttatcaggataccagtccttagatctagggcccaccataatccagtatgaccgcaccttaacttgatcatctgcaaagactctatttccaaataagttcccattcataggtaccaggggttaggattttaacatctttttgtgggatgcagttcaacccacgataccatctttgcccatctctggctctggcgtgcagggacctgttactgggcgggtcactagggataccacctctcacctctctgggcacccattggtaggcctgtgctgtgcccagcaccagggggctggctgaggcctccttgtycagtggctcctgtgtgcctctccacttgggggttgccctagagctgcccaggaagggaggaggcacctcagggggggaaggacagcatgagtttccatgggcttctcgtcacccacacagagtgacttggaataatgcacccagtggctgatctggggccctgagctcgaggctgcagccagtggcagctccaggccctgcagtgtggggacagcgatggccaaaaagagcacatgccaaaggccctgccacttaggggactttgggaaagtttgaccaagtttccttgtctctaagtaaggaatgataattggtcgatgggattgttctgagatgaaagtgctggtccttgggaaagcttcagctgaaatgtctttttattttgcccttcactcttttttttttttcttcactgttgtcgcccaggctggagtgcaatggtgcaatctcggctcattgcaacctccaccacctctcaggttcaagcaattctcctgcctcagcctcccagatagctgggattacaggcgcccgccaccatgcccggataatttttttgtatttttagtagagacagggtttcatcatgttggctaggctggtctcgatctcctgagctcaggtgatccacccggcttggactcccaaagtgctgggattacaggtgtgagccactgtgcccggcctgcccttcattcttgacgaatatttcgactgaattcagaattctgggtttccaggttttaacattttctttcggcacattaaaagagtcttctgggtgccattatttctgctgacaagtcagcagtctctgagtcctggtgtctctgaatgtaatatgttgtttttattctagctgctttaacattttctcattatttcagtttccagtagtttgcagtttgaccaggatgtacctgggtataattttccttgtgcttatcctgcttggagttagctaagcttcttaaatttgtaaattgatgtcttttaccaaattgaggaatttttcggtcattatttttttatgtgttctttttagtatatcttctctctcccctccctctacaactccaatcacatgtgtgttgaaccttttgatatgcaacaggtctttgaggatctttttctttttaaaaaaattttttttctctctattcttcagactgggtaatttctattgaaatcatcttcaagttcactaactcttctattttctcagactttctgttaagcaatttctgttaaggtgtgattttttttctgtctcagacatggtatttttaagttatagaattttcatttagttcttttttaaattgtttatatttctctgctgagatttattctttcatgaagtatgagccaattttctttacgtcttttaacacagttatcatagcaagtttaaaaatctctatttgctaattgcagcggccagcacatttcaggtgagtttccactgactgtcttttatattaagtatggctggttttcctatttctttgtatatcttcatataattttggatgatatcctgggtgttgtgaatgatagcataagagcgactctagatttttgttacttctccccaaagagcatttttgttttgctttgtatttgcaagcagttaacttggctgaaagcaactgtaaaccatctgcagtcgtgagtggtgggcagtggcaggagcctctgttcacttcttctagccctggctgggttgcagctgctggaagtctgtcctgtgtgtgtgtggtttggggtcagcccaagacatgggcagagtttatttgccccctctggggatctgtcttggtggctctcttcattccagaatttactcccacatttttccagctgctgcgagtgtctcagactctgtttgctgctctatcaagccagtgagactctggctttccgtctgagttctggcagccctatgacacaggctgggacttgtcctcaggctgtgaaagccatgagaccaggaaagccaacgccggctgttcctcccaattgctgaccgcctgcagcatctgctgcggcctcaccctccgatgccttcagagtgtcattttgttgttgttttttcaagggtttatagtctatagttatctgtgagagagttgctgcactaggagctactaggctattaccggaagcatttagagcagggcctggcagacagtaatcacttaagaaatgtgagttaatgtggtcaaccatagtgcggcgctgtttcctctggtcaaggctagagacaacgtggacacagacagcaccatgcaggaagtgtcagtacctagtgaagagcacacaatggccttaagactgcacttgtaggccgggcgcggtggctcacacctgtaatcccagcactttgggaggtcgaggtgggcagatcacttgaggtcaggagtttaccaacctgaccaatatggtgaaaccccatctctactaaaaatacaaaaattagctgggtgtggtggcaggtgcctgtaatcccaactactcgggaggctgaggcacgagaatcacttgaacctaggaggtacaggttgcagtgggccaagattgcaccgctgcactccatcctgggtgacaaagtgagactctgtctcaaaaaaataaataaataaaataaaagcctgcagttgtaggagagaccacttaactgggggagggggaaaggggactatccaggaaggcttcctggaggaggtggtattggagctggaaatgtcagataatccaagatgagaagatggactttcttcacagaggtggctgccaaaacaaccaattttatggccttactgatgagaatttctggcactcaagagaggcaacttttcctaaggcaaatcctgaagacatttacctataaaaatgagtagacattaaacatccactagctccttcatgggccagaggaagcagtgctcaggcaagaggggatctgaggtctggtggtggttctgtagatgtttgcagggagcgactggcgggactggtaggcgggaccatccacagtgaggttgggcgcccagatccatcgccacattgcagagcttgtggctgtcaattctgtggcagccaggagcacaggtgaccgggagctttagtttctgtccagtacattttggtgattcattattcctggagtcctcgtcttctcttcctccctcccagaccctctcacctccttgtctcacttgaacctctaatcctgggaaggctccaccataagcacactacagataatgaaaccaaggctcagagaagttgtgtggcttacccaagaccacacagctgaagcactggagccaagactccaaccagcacttctgtctgcaaagagctcatggcgtagtctctcatggagggagcctccatctcctctgctgccaaatggaaatcacagtagtgtctcccgcaggggctggcgtgaggttaaaggaggtagcagatatgaaaatgacatataaattgtaaattgcccacagacattagtgtgtgttgcctggtaaccacccagcatttgtggactgcatttacttgggatagcaatagttatattggcattttaatttaatagcattgttatgttcccatcataaaaataatatacactgtgtgtatgcccaccctcatcaaattatatacattaagtatgtgcagcgttttatatattgattgtacctcaatacagctattaaaaaatgatgtaacttcattatagaaaatctagaaaatatggacaagttgaaagaggaggaaatattattttgttccagctaccctaaagctacctccgttcacattttggagaattctcttccagcttttttccccactgcccagtttctttgcttatttcacatggctataatcactgatgtagatcattacaagcattttatacccaattttatatcccgttccttcaccctcagggggttttaatatgaaatagcaagaaccagcgccccatcggggaccctagcacagctgggctgggtggttctggcttctgcccagatgtgtctgggtgtccctgggccagggcaggctgccccacggagggacaatgtcaggacagctgggacctgaccctgttctgactccatgcccggtgggcgtttccgtgttcccacagagctgcttttcttatcttgtctttttaagttcagtgacatgtgcatctgtcttgatggatggatggatacgtccagcccgtcatcccacttctaaccaccctctctcaatcttttctgttgcctcaaagctggcattgactcatggctcagaaggccagggcaggccaggagctgtcctaaaaggcccagactggaaaaagaaaaggcatttcttggatagatatctctggcctggctactgcatacaggcaccatttgaagccgtgaattaagcctgaggctcctgccctcatgaagtttacgttctggccaggaagagagcctacagatgataaaagcctttacagaaagaaaaacatcagattaggttggggcagggatggtcagggtgtcctgtatgagggtgctggggtgcagacagcagtttcagcaaggtgatccaagctgtgctcacgggaaggcagcagtggggcagaattggaagaataggaaggagccatgctggtacccagggaaagccactgcaggccgagagagcaggtcttgggacagcaggtggccaggacttttgggaaagggcaaggagcctggtgtttggaggggaataagcaggtggagagcagtagatgaggctggggaaggggaggaggtaccttttgctgagttaaaaacacgagaccccgtgcatacaacagtaaatgttttcatcatcttgcatctgcgattctgtggtctgggcttggctgggcttgccctgctctgtgggtcagtggggccctgctggaggctgggcttggccgggggttgggggggttcttctgcacccactcttcctcctccccctcaggggccagtgggccagcctgcgcaggtcctgctcctggtaccgccagaggcgttgatggcaacccccagtacctaagcccctttgcatctctgtttgcggcttgtcgaattgccgttctgcaggccaaagcaaaacacagggacaagcccagaatcaaggtggggggcagagaacaggtcacctggcccaccatgggaggggtgaaggtttggctccagatgcggcccacctgggcctcggcaggtgggggctgcctttagaacactctgtgcttttctctgagtacacaatgggagccgctggagggttttgagcaggggagtgccaggatcagtcctgggtgttaccagcctctctgctgccagtctgttgaggctacgtcatagccagggagaaagagctgtggcagtcatccagtgagaggtgggagagtggctggcatcccgatatgctgtgaaggctgagcaggcaggattggatgtggggtgtgtgtgtgtgtgtgtgtgtgtgtgtgtgtgtgtgtgtgagagagagagagagagagagagagagagagagagagagagagagagagagagaacgagaaactccaaggcttgggcctgagcaagtgaacaatagagctgtcatcacctgagactggggaagctgcagaggatgaggctgaggagggatcagaattcagttatggtgaccaatgaagggttttgaatagagggacacagggatgcactggctcaggagtttggtcatcacgttggccgccagagggagaacagaggctgggacagggaaccctgtggagtgtcctctctgtggctacagggagcaacggtcagatctgtggctggtttgaaagtagatccattttgctaaaggtggtatcacagattttggagactacagaatccaggccaggagagcaaagaacgacagaggtcccattcacagcagcgggccatcccgggctcctactagcttttgctgcttgggcctatggggatggtattctagaactttccatcttccaagacatactggaaatgtggatttttataggaaatcttctacttttcaaatattaagaatgacttcaaattaaaagtaataatcattttggggctgcagtaaaacttgttggtggagcaggcctcaggcttcaggttggtgagctgtcctccagtgggggtttctcttccaggctagggccttgtcctcccctgctctcccccactcccagggctggccacccagaatgtgcaggccactcatctctgtgactcgtagaacctgagaatgttggtgatggaaggggtgttcccctagttcccacatgcaaaaagtggggcctggagatggggctgtcttttttctccaaacacttgcccatccaggaaggccagccctgggcattgaagactccaggtatgggggtgactccataaggagaggttcctgagtgacctgaagaaacatggttgggatgaagtgcagggagggtcagggcaggcagggctgagcttcgcttctgcgagcagagattagagagcgatcccagaggcctgcctggaggtggggccacagagcaggagtctgaaggagagggggtaattcacctctgtcctactgtgtgggaagccctgagtgggcccctagcagagggttgccttgcgcttatctatggagatattgggaagggcttccctgacccaggttgctggagcatgggggcctgggtggcaggggtaggtgtgggagattcaagacaggaggatgaggccacatccaggtggcttcagataccaccgtgagtcccatagagtgtccagttactataaagtctgccagccactgtggtcgccccaggcctgtgccaagtcccgagatctctgccctgcagccagtgcagagggaagaggagggcaggaggggacaggttggtggcccctcgccgatgctcctgacccctcaggctccctgctgcctcctgcccagggctgtggggcctcggggagcggggagctcgccattgctgtggcactgttgagcacccgggagaggcgggttctgacagccaccccttcccgtcttgctgttcccctcccagctgacatcatctctaccgttgagttcaaccacacgggagagctgctggccacaggtgacaagggcggccgggtcgtcatcttccagcgggaaccagaggtgcgaagccctgggtctggtgggaggtgggaggggtggggagcagagtcaggtgggaggcgggcatggtgggaggggataaggataagggggataaggggtgatgcggtgagaggtgggaggggactatactccaacccacaagagggcttttaactgacaggaggtgtggggtgtcttgcctggatcacatctcccaggggcatggattccaggggtgggaatgggatgctttggaatgccgaggtgggagtggcgggcggtggtagggggagtgcactgggtgggctggcaaagccaggttctgagccccctgccctcagccgatgtttgcggagagctgcctgcatcccatgctctgtcctagaccctggatgaatggtcaggatgtggcacaggcagctgctgccccagcttgtgccttcagtgaaaccttcctctgcctgggtgacctgcgttgtcctggccagactggggagcgggaggactccactcttgcccagctgtcacagccctcagactgagctaggtggtttttgctcaggcctgggcccttgtcctcacccctgtcctcacccccacccccgggggcttgttgtccagcagaactctgggtaagacagcagtggcctggacaacctgcccaggaccacgtttctggaacactgaaagagcctcgggacaaaagcccatttactgagaacacttgctaagtattctggagttgcttctggtttactctgcactgctcacctccgagggccagatccacacccctgtgacagatgcggaaactcagagaagccacccagctagtgaacagcagacctggctgcccgacgccaggacagtggccagcaacgtcacacaccacccggcctcagagagggcgtctccccccgcaaacacggactgtgcctggaattagatgcagaagacatccaggaaaccaaggaggagggaggaggggatggagcaggagaggcaggggaggggaggggaggaggagaagagaaggtggagaaggggaggtaggagggaggggaggaagccgggggtgaagagaggacctgcggaatcatctttcccccgattcactcctgagcatggcccacgaactgaggatccttgagtctgactttatcttggtggctcacataaccctcctcacggtgaccctgtgggtgtagtgcagtgcggctactcccctcattttacagaggaggagcccagagtgcagagggttaaaatagttgacaaggggcacatcctagctgccaatcagttcctcaaacacagcactcggccagcacctgtgggcttcaccagatgcacctgtggggtgtcccaccaggcactgcgaagctcagccttctttttgccaagctgcagaaagagatcggattggttgatgcgattttcttcattttgaatttttctcctttagtgttttcttccttctagtcccactgacctttgtcatagcgagtgctcatgggaagtgaagtgaattagaggcaaaaatattggagaacttctaagcttttcaaaagcaagacagggatagaaatatagatacttaaaaaaattaccaggaaatcttgaaattgttctgggttgaatgtctccgtttctccctggttggggtttcgtgttgtcgtatgagttgaactctctcctgggaacgtggtccctggcgcacacagtggactcaccacctgactatgctgggagctccacggtcaagggtctttattagaaggagctctgctgcctaaaccaggccaagcactgctgaggaaagccggggacagattccaatccaggtctgacattgccagacattctgtcccaatgttaaagtctgctcccaaattcatttattcctcaagtcaggcctgagttggacacgtggaatccaggagcaagtcagggcccaagacacgagggtggccagagcctcaagccttggcgtcaggcaggcctcggcagaggggtgctcgccggagagtgacctcacggctctgggaagtcacttcccctctgagtctctgtttgctggtctgtgaaatggggagagtcgtggtgctcaagcagaggaggcctggagggtagaatgcgatggtgcggggaggacttagtggggtgtccagcttgtagcaggcgctggggaacattcgcattagcaagttagccggctcgggtgtgagtgtttcatgggaccgcacgggagcggggcttgtccctggcacactgcaagtcatgggccggttaagctgcagagagtttcatttgaccctcgaattggatccctggcacggctcggcacttggtcccacggccggccctgctgggtccccggaggtcctagccgtcgccctgcaggtcacggtgctcaggccccttctccgggtttccctgcagagtaaaaatgcgccccacagccagggcgaatacgacgtgtacagcactttccagagccacgagccggagtttgactatctcaagagcctggagatagaggagaagatcaacaagatcaagtggctcccacagcagaacgccgcccactcactcctgtccaccaacggtgaggcgctgcccggcctcgcttgcatgggcacaggccgtagatgtttctaccaaatgctggtttgtatttcacttatattgtgagcatttttctatgtcttcagaacgcctagaaaataatatcctttttgagcagcatataaatcccatgagtgagtatgccagaccccctcagacgctccctgtgtctggacacagggctgcccccctttttgagcagcatgtaaatcccatgagtgagtatgccagaccctctcagaagctccccatggccagacacagggctgccccctgctgtgaatagagccatccgagccatctttgtaggcctttgctttggggtgagggagtgtgatggacaaggatgcaggtcgcagacacgggggatatttgacacaaagtgaacccctcctccccacgccgaatccagacccctagactggggctgtcaccgcggccttgcggggcagtgggaggagccttggtttaggaccccggccggcctcttaatcctcttgcctcagggaggagcatgtacccctcaggcaccggaggctctcggcctgacgcctgcttcccaggctccactctgagggagtggctggggctgtcctgcttggctcaccatgggcctgggtcccccactcacgccccctgcccatcctccttcacctgggccccctgcggacccaggcagcctggggtcgggtaggacacacctggattcttttttttctttttttgagagacaagagtcttgctctgtcacccaggctggggtagagtggcgcgatctctgctcacggcaacctccgcctcccgggttcaagcaatttttctgcctcagcctcccgagtagctggaattacaggcgtccaacaccatgcccagctaatttttgtattttttagtagagacggggtttcactacatgttggccaggttgatctcgaactcctgacctcgggtgatccgcctgcctcagcctcccaaagtgctgggattacaggcatgagccactgtgcccagccaggacacacctggattctgtccctgccctgcctcccttgggcagtgaccttgctttcctgagccccagcagggtggacacatctcccttctggggtccgtgaaatggtgcccactcaaccccctgagaacagtaaccgctgcacaagcaccagccaggcttcgaggtgctgtccccacgccttccccgaggacgtccacgctcctcagcggggcctccaaggctctcccgaacatggctaggcgcagcttctgccagcccgcctgtgcctgaaggctatcctcgccctgtacctggccaactccctcctcatcccataagccttgctgggtgcccgccttttctgacctctccactgagccaggcatccctcctgtgacaccctggctcctcttaactccggttcaccccctctctgggcacctgctgttctgatggtgttctcccgccatgcacctcaaaggcaggatctgtgtcccctaaggggccctgcccagcaaatctgtcacctgagccagcagagcatgagagatcctcccgaagggcaggccctgggggacaggagcctcgtcctgtccccatgcacctgctccttttgaaaatatttaagaaggacgtgtcagccacatgcggtggctcatgcctgtaatctcagcactttgggaggctgagatggatggattacttgagcccagaagtttgagaccagcctgggcaacgtggtgagactctatctctacaaaaaaataaaatattagccaggtgtggtggtgagagcctgtagtcccagctactcaggaggctgagattgaaggatcatctgagaccggtaaagtcaaggctgtagtgagctgagatggtgccactgcactccagcctgggtgatgtgagtgagaccctgtctcaaaagaaaaaaaaaaaaagacatgtcactttcttcctgcctctctacagaaaaggtcatctttcagcactccctacactccgctcccctcaaagcttcccacctgggtttgaatcctggcttcactctttcctaatggcagggccttgggtacctttcagatgctccactgaccccagttctcagttctcttcccagttccttctcatcaacatggtgctatttgttttgactcctgtagtagtcagggttctccagagaaaaagaatcagtagggtatgagagtgagaaagagaaagggggatttattttaaggaagtggctcacctgactgcggaggctggccgttccgaaatctgcagggaggccgaaggctggaggcctcgagacagagctgcagttctggtccaaagccgtctgttggcagaaccccctccttccccaggaggtgcctcatgatttcaccttctaaagtggatcttctcttccatccggctgcttaattatgtgactcgctttattttgttctgtttttaaagataaaactatcaaattatggaagattaccgaacgagataaaaggcccgaaggatacaacctgaaggatgaagaggggaaacttaaggacctgtccacggtgacgtcactgcaggtgagctccggtgagggggaagcaggcacacgcctctttattacacctgaggattttagggctggaaaagcctttgagatttgagccagagtcaggtgcagaccctggttgggctgctggctgctgggcggccacgggcgtcttcctgccccttgcctcagtttcctcaccagcagcatacagatgaccacgtttcttctaggcttcttgtgagcatgcaggggtggtgtatggctgtaaagtgcttttcacctatagctgagggtggagagcaaggcagataatcccttgttataagaagggggaaactgaggcccagagagagacagcaactcacctaaaactgctcagctgagtgagtaggcagaggcagaaagagaatgggtcagcagagctttgtggctcctgagtcctttactgagggcagaaggaagcctggaccgtgtgaggccttcagcatctacaaaggcctgaaacagacataccctgaaacagacacacacgtgcccagcatccagtgagccgccataaatataaagcagtagtcagtgctcttcctgagaagaagaaataaatgtttaaatacattggcctaaatgactggcaaattgcacaaataaattctgaggaagtgggcagccgattagcttcgatgttgagtaatgagggtgctggtgccccctgccccaccccccgtcccaagaaagtttatgagggtttcatctagagggaaggatacagcgatcacatcttgatcaccctggtgtttggggcagagccagtggccttcataaaaatcctgtttatgtccctcccagcgttggagatttttctctgtaagtcacacacatcccaagacacttaactgtcactgagaacctgggtgccctggtctgtcctcatcctgcctctcaccttgtttgcttccgatttcacccacgccacgccaaccctgcacagcattctacccaccctgcgcagttcacactggaggcagctgtttactgaggagctgctccacgctaggccccgggctagcccttggggccctagaggtgagcaaggttcaactcagtacctgtccccaaagtgtccccagcccccaaggaaacagacatggaatcgccatcacactggcgctcagtgcggtgacagaggaacacagagaggccgtgggcaccccaggaggcctttctctactttgtcaatttggtgaactcccatacatcccataaaaccctactggccttcacctctgtaaagcctttctgcgcaggccagatgacaattgtgatctttcctgtgtcccctcttgccttttggatccgacactgtctgtccttagtgtctgtatcttccttgcccctcatcttgactagactgtggcttctgcggaggcagcatgttttactcatgaaacccccaaaccttcagggaaggtttcctttctctcttcttctctttactaaaaaactggttttgagctctggtttggtggatggcagcaaatggctggagcactggatgaactgggtcccaaggccgcctggagaaaacagaaagcaatgttgccggaaggaagcaggaggtggactgagatgccaagactttgctgggccagtgatgctctgcccttctccagagcagctgggctccctctctctcctgagagtccagggatccccagggtgggcagtggctgcaccccgaaggaagggaaggttagcagcatagctaaaatatctgacacaggaatacgtgcagggaggatgccttggaggagtnnnnncacactaacaccaactcccaggcacacaacctagaagcaaagacacagaggcaacaatgcaagaaccacccagagccaggctgtcactcacaccggtgctgcgtgcacacacacacacacacacacacacacatgcatgcacatacacgtatactcagtcatgcatgcacactcacatgcacactcatatgcacacactcatacatgaactctcagccatgcacactcccacacacacaggctgggtgccctggtgtgtgggaaaatttaacgtgggctgcagagctgcctctgctccaaaggagctcagtgcttgtcactccgactgcagcaccgaggctgtcacctcccatctcagctgttccctgagagctcagggacacagcccattcattcattctctcacacattcactcattcactggagagtctctctgctaggccttggactgagcgctagagagatggagagataaacaagaggtgacgccttgctcccgtagctgatccctagtccactgggaaagctgatgggaccagttgacagcctcagggttggtcagggctgtgcaagggaccttcagggctgtgaaagctcagtgtctgatgccatggcaaggactcctcgcaccaactgtctgagatgaaagttcgtatgagtcaggctaactccaccgctgtaacaaaacaacaccagcagtccagggccgagcacggaaatgcatttctcactatgggaaggttcgatgtggatgtgctggttgggacacagctctcctgcaggatccttccatccccatcccatgagtctcagtccctaaggcttgggtcctcaccttccaggtggggaaggtgctcccgctgcttagctgcatcagggccaggtatcactttgctcacatcccattggtcagaactagtcatgtggccatgtctgggcacaaaggctgctgggaaatgtagtccacatgtagacagctgtttcccagtgacagcactgcattgcaggaggaggtgaggaggcctggcagacaattggctggctctgccacagtatctttttgtttcattttagagttgaggaaattggggtataggagggttcagccacttattcacagacccacagcaggtaagcatccatgcctctcttgcagggctggtgggcggcgggcatttgccatctgtcactgcctatttcagaccacggaggatcaggatggacgcccacccctgtgttccagcctccggttgggatcacatgctcacggcatgctatgtcctgatgccaccrcctggccttcacctcttccctgcctcctcatcctctgcaggtgccagtgctgaagcccatggatctgatggtggaggtgagccctcggaggatctttgccaatggccacacctaccacatcaactccatctccgtcaacagtgactgcgagacctacatgtcggcggatgacctgcgcatcaacctctggcacctggccatcaccgacaggagcttcagtatccttcactgtggcctnggccagtgcctcccacgggcagagtagcttccgttgggtggtgggtttggtttgattggcagacagctggtttggggatggctgcattgtttaacttcttcagtgaggcacctctggctcctagtatgcgtgtgaggcccagatacaaaatcatgtcacgtctgtttctgaaaaccgcaaagtcgtggttgctgagcattgcacccatcgcctcctccagcatggccatgatcccctcatcctagggcctcacaaggggccaggaaggagacagagttcagggttcagctgctctggacgagggactgctggccttgctcagcgtccactgaaggcgcctcggggccctccccactgggacccaaggcaggctgtgttagcataggaggaccagcactggggccccggccagggcttcgggtgacaaccagggtgtcagaagcccagctgggttggggtcctgagggccccctgctcggtgttctcctgtgtcagggcaagctaggggaagcagcagcattgacgattcccgccgactgctgggaagagagctggtgatgacatgagcaccagcctgcaggcagggcgaggcttggcctgccggggggctggtgggcgggcaggcaggttgctgtgatgtcactattctgcatacaaccaatgataataataattataataacacacagtgagactctgtgtgccaggctctgtgactaacatcaacaagcattcattttaactcatttcaccttcacaacaaccctatgaggtaggtactattctaagtctcctttacagatgaggaaactgagtcacagagcactcaagttaacttgctcaaagctcacagccagcaagtgtcaaagctgggcctgaaacccaggccctcaggccccagagtccccgcttctaacctccataccacactgccctttgtggaggatgccacccaggtgtgtcttggaaggggtgggaagaccctcctgagagcctccatgcaactggatggcctcccgactccaggcagcctgggcacccagcagtggtcaggagtgggctgtgtccctcggagcagtagaagccgggatagctgtggggagcaggggaggcagggagcattccaggaagggtgaaagagagtaacaaatggcttttgtggtctgagatccaggcacagccagagacccctgggcagggagacccttggaaaacagcgggaagggaacagagcaaagcgttggatgtcccctgagccagccagagggtgcggggcacatgtgtgggcttcggaggcagaaagtctgtttgggtctgactgccaccttcaagctttgtgaccctgggtgagtacactcacctccctctccgagactccgtcttttcaatcatgcctcctcatagacctgtggggtgccgtcagtgagcccctatgtgtgatgtgccgaaccctaagtcagcacttggtgggtatcaggaagcatcagcatctctctcccttctcttgtcccaaaggcatggagtgcccagagctgggaggctggattgggccagccaggaaggttccaggaaggattgggacctgagctgctttgaaggcttgaagctcagatttctctggacatagagggctgggctggacatctgtctgaccctccactgaagccgaaaggacatcgggccccaccagtccacagtgctcatagcagcagttggttagactctttctgctgcacgagcatgtttgatgtggataactgtaagagagctgcgggccaggtggcttcaggggctggagcatcgtgggtgatggctctctgggctgattgcctctgctgtcttctgtgtttcaatctcaggtgccagtagttggggccccaccctcccagaataaagtccaggagaaacgagaccagttctctctgaactgtcccactaggaccctaaatggcatcttgtgggctctaattgggggtgaatgccttgttgtgattggccaggccggactcctgtacctctccctggtgctggagggagtcagctccgcccccaacccctggatgtgggggaggggcggtctcctagcaggagctccaggtctgcaaatggggagggtgtggggctcactgagcggcctggaccgtaccgtacagtttacaaatcagcctgccgttcgttgtctcacttggtgctcagagacagtggtaatttgatgatcaccccatttcacagatgaggaaacggaggctgggggagatgagggactggcccagagtccccagttggcaggggcagagccagagctcatgctcgggcctccatgcctggtccagggcttttgccctggcctcggcctgccccctgcagccctgggacagtggcagccacccttctcgagcacctgcttccttcctgccggacttgggtggggatggtaattgctgctcggttttcctgtctgggctgctgtgaggattccgtgacactatggatgccgaaaccctttctaaagaaccataaagccctagagtatggaagggatgctgcttcatttcatttattcaaccattcattccccacgcctttctactcccaggcccacactgagccctagggacatggggatgaatgggagaaactcaacccttccctggagggctcaggggctgctgggggacacagccccgccatggcagtggaagtgagggagtcatccttgctgtagcccagggtctggacaagctgctgtgggggccgccaaggaagccagagaaggaatttcggtaggaagctccctgggctcgaggaggctgcctgagggcatgggtgtgcaggaggcggatgcatgaggaggcagaaggtgccggagctgtggcactgtggatggtgcaggcagggggctttctcttggggtaggggccccgaatgccagtcattcacaaaaccccccacagctgtgcctgcctccacatgccacgtgtgcttccctttacttttcccatttaaaaaaaattgttttctttaagtcaagttgctttatttttgccgaagtaattttcttttgaaagggaattgaatttcaccatgataagttaaagtatcacataccataaataggaggtagcctcaaccataacggctgtgcacacaaaatggtgggaataaaatacaggctcagccctgtctgttggaactttctgtggtagtagactgcttcgtctgcactcatggggcggcagccaccagccccatgtgcctgctgagtgcctaaaacgtggcttggtgactgaggagcgacatcgttaatttgatttagttccagttaatcatacttcaaacgaaatagccacatgtggccagtggctactgaacaaggacgctgcagcctctctccaggttgcccttagaccctgaagctggttctctctgaaaagatggaggtaaacatgtgcttcaatgttaagggggagagtgcaaaacagagacatcccccttcattcatcagaaggattgaatcctgtctccccgtctgcatctatattacattactggaggctgtgccgacttcccacctgcctgcttccagggaacgggaaactatgaagctattgatgtactgtaaacaggggcgtgtcactagccagctttggaggtgtgtatgtcgtgggtgtgtgtcgtgggtggattggattccagcagacaaggctggcggcttgggaaccagcaaggagacccggggagagagagtgagaggcagggccagagcgtggccacgggtgggaggcggtggaggaatgagattgtaaggatggaggtgtcatagatttggattgtatcataaccagttttggatcggggtggccacaagacattcagacaggttggctgtaaagaagaaaagggggttggttcatcccaggactgcaaagtcctccctggccgttaccgtgagccttgcctcctgtcccgtcccgaacacacagattgtgccctgtccgcatgttgcagagcacaccatttaccagcaggtatttattaagcacctactgtgtgccagccctcttgggtgatgctggaactagataaagattctgcctccaggagtggacacgtgataaaccattggtaaaatacccggcattggagcaggtggtccgtgggtgctacagggagtcggtggcccagccagcgcagccgtgtacatttctctccagccacgcctgctgctggctgcaggcctcaggtcggtggagggtaagccaggggtgtggtttttgccaagtgaggatgtagacacggagctgatggtgtgtgcaagagagtgaaaataatgattattgaccaaagaatctgagccaggtgagggacagtgacaagggaggagtgtcagcagatcgggggcccctgtggatcctgggaacatccctggccgcacaccctgccgtgtaactcagtgtcagatctctgtgtaacttcccattctgtgaaaatgattattaattcatcaaaacataaagcaccctggccttagtgatagaggagtgtgtgtctatagcatgaacaagattctgagctgggcttgaggcagccctgatagggcaacttaggcatttggcaggagttgagtggagtctccaggggcacatggtggccatggaaacctccccgattggatctattcattggggaacctcagtgcacctggtgctccccatggcctgttgccgcttggctccccatggggtgctgctgcagtgacaagacacagatccagggctcacaccacagagagaagccttacttggcagagaaagtgtgaggactgtgggctgcaggtgctgcgatgaggactgatagcagccgagtaatgagaggaggcgtttcatttctgacatgggaggaggccagaaaggcagctgagaccccatgcggagctgggaattgctctgaatgtttgagataccttctaagcaagtctcagtggggtggattctggctgtcagagggtgacagttctgatgagtcctgacaagaaaaacccagggtcaggaggcctgtgtccgcgcagagccaggggaaggcttgggctggacttggggtttgcatccttctgacccctgctctctggggtcctcactcggcccgatgacactgttcagtcctccgtgcccactggaggttttccaccatgtcgcatctcctaagtccaatcactaacacttcccgtctcagcaaaatggacttttagtaaccatatcaagagaaaggaaaatgtttcttagtaaaggagaaaaagagggcattgctttgactattggacaatgagaaaggtgactcagaggacatctctgctggagggagggagcctggtgatactgaggctctgtgacctgtctcggtgtagtcaccaaggttcaaggaagaggagccctcccagcctcctccctctctctcaccctcagaatggaaagaggtgcccagccctctctactctccccagacacttgcctgaatcccgccctgtcctgcccagagccttctgcacagctgtgaaattggctcatcggaacctctggtctgaccatgtcagccccccttaaaactctcacgggttcccctctggcttctggaggaagcctgagcccctccacagtgccctgtgcctttggtgaccagcccctgcccacctctcgggtcacactggctcactccttcagctgctgggtccctggcatggggcagggtggggtggagcactgccatccgccatcccttctcctgaccatgccatctgcctctttggcctggctgactcctgtttgcccacttgcagcatctcacagcccattatctgggacagcctctgacctgtccctgccacacccctcctaggctggtcagagcccctctaagcctgcggctccttggctgcccccggtcacactgagtgttttgttactgtgacagcctcctccattttatggtggaagccctgagattaggaacccagggtgaggcctctctgcatcccctgctccacataggacttggcgataggaggtacttatgaacggttgtaggtggagtgggctggaagttcaccacttccatgtggtcttaggatacagacacctcctttttgatctgggcgcctggggaatcagagaccccagaccctaaagtgcaggagaaggagcccgggctttgggccctggcgggcctgggctgagacccattgctcagcctgctgggccatcagcttccctgaatataaaatcagaaaattcccttttataaggatatttgtaaggaataaagaaatctttcacataaagagcctggtgcctaattggagcccagcaagaggatggtttattttgtctcttaaacaacagcccttcaaacatttaagattggttctcataaaatccctgcatctctgatggttaaaactccgttttccacggtgcagtttcaacgtctgtttaagcaacctctcgcgtcctttaataccaacattttgagactgttaacacagcactgggtgatttttcttgagtctgatgcatcttaaaggtaacaaattgcacaggaggtctcagcggatggacctctgctttatttttagcacccagagaatttaaaagacggttcgattgatttttctctcccttagtgatttctcacggtgggaactggcatcacaccaagcctcggcctggagaatttctaattcccatttacagagcaggtccgcacggccaggtgggctggaggggggtgcagccattcaccgcaggaggctcagaaccttcatcagcccatgcctgcctgaagactcagcttgtcttctgtgcagagaacaaaggagggagcagacacagtccctctgcatgcagactctgtgctgggccaagggaccctgagcccaggctctacccctgcctctgccactgaccctgatcagtcactcagtctttctgcaccccctttatcccatgggagtgtgtctcctttaccaggagctgtaaaggtggaatggccgatgagtatggggcctggtgccgtgtagacaagcaaccctcaccagcacatgacccactgtcgggtaccttacaggccacctcttcttcctgcccttggtgctaatttacagagaaggaaactgggatgccctgctcagaaatgctctgttcacggcatgtcagatacataccactgatggctgggagtctggacccctgggttcttactccagctctgccactacctgcccacggttctgtgcattcattggttgattcagcagcattggccgagagtcttctctgtgttgagcattggacttggtgctagggaagcagacagggtctctgccagggcctgcgagtacagttgtatgggttgtctgctgcccaaaagaggtgatgatccaagccaagcaccttgggaagaagggacttttttgtttttcagttctgcaaggaagagctgtcttttcataattggcacaaaggcactttctgagtgaatggggatcctgatgctatccaggtgaactttctaggtgagcaaaaagatagtcttcttcttgctcaagcctcgatgagtcctgtgatcccatcgtccaaatgcacccagagcccacctgcctgtctgcccccagcatgtgtttaggccactgtctcactctgatcatgatggtgacctgactggcctcccttcttaggtctaggagccagactgccagggctcagaccttggccccaagacatagggactccgcggcctcgggcagctcacttgggttctctgtgcctcagctcctcacctgtaaagcgaggaaaagcccctgctgacaccatacggctgcttgaggactgggtgctacgatgacatctgggaagcctttagggcagtgcctggcaaatcaccgagctgcagcatcattgcttcattagctgtgtttccccaggtgtctccttacagtctgttctcctacaacatgagaaaaaaaaaaacgacaagatcatgtcactccttgctcagaacccttcagcaacttctcccaactggaagtaactccgcccctttctctggcctccaaggccctgcaggagctggccctggcccctccctggcccacttcctcctctctccctttgctccctgggctcagcgttcccggccccctgctcttcaccatatttccagccatctcctgcagcagagccattttgcactggctgttccctccgcctgtgaagtttctccatcctcatccactcagtggacaccttcttaaagaggcnttccttgaccagtctgtcggaactagtccatcccctccatgactccctcgagccccttccctagaggcttggcctctacctcgttccctgttcatttcccgcctctccctcctggactggcagcttcttgagagcagggaccttgtttgatctacctggaataatgcctggtccagagtagatgctcaaataatggaactagtgtaatacagtgagatgggtgttaggataaggtggggaggaccccggcctccttagaaagtccttgatggaaaggcattgacactgaggcttctgttctggacctgccacgtggtcccctggcagtttgcttcaccttcctggacaccagtttccccttctcttggctggactggtgtcctgaaactggctttcatttagacagttgggctatccttgccgggtctcccttgatctggctatgtgtgaccttgtgtcttgccagctgctagcatgacacaaggtcacacacagcttgtaacagccaggtgtgcacatctcttcccaaatccatgttcatggacatctctttggcagcttgaatttggccatggtggtagtatttctttctttttcttttctttttttttttgagatggagactcactgtgtcgcccaggctgaagtgcagtggctcaatctcggctcactgcaatctctgcctcccaggttcacgccattctcctgcctcagcctcctgagtagctggggctacaggtgcccgccaccacgcccggctaattttttgtattttttagtagagacggggtttcaccgtgttagccaggatggtctcgatctcctgacctcgtgatccacctgccttgacctcccaaagtgctgggtttacaggcgttagccaccgcacctggcctggtggtagtatttctacagatatctacaaacactacaaaatcaatctctcccctccccagaagagagggtattgttcaagttaccagcacacaactgctctggggccttcacaaggccaggcagttggtcagtcagcctccancatgactgagggcttcctgcgtgccatggcnctgtccatggtgctgaccccgttgtgagccattgcgtcgaggtcagcatctccacctcccaccccactgtcactacttgctgtgtcacacggtgttctgagcacgctgccgatgttaactggttgaatcctcccaacaacactatgtggtaggcgttattaatatcccccttttacagatgaggaaacaaagccacagtaagtttcagtaatctacccaaagtcaaaggggaaagggggtgcaggggaaagggggtgccagggctgggattcagatttaagctgtctggatgcagaaccatgcacttaactgtcatgctacaccgcctctcagtcatgatggtaacaaccccagttgcagcctgcctggtgctggggtgaacaaatgcaagatgccgctgttgcatttcagtctcatgaccactcgccaggtgggggctgtcactgtcccaactcagcagaagtggagatgggcactgagaggtgggctcccttgcccaaggcccctctgagccaagtgtccttctgatggcctgccyagctttgttgagacagccgtatttcttggttcttgctttaggtcctgtggaaagacaggcccaattgcaggatagatgggaggttgttttgctatgagcatggctgtcagtagctgtatggcagtcctcacgtttgccctgcaggtggcatggtagaggtgtgcgaccctggcctgcctcctgtcccttcttgacagacctccctggcatttctagaactcgctccctgttaaaatcctcttgaccccaggcctgagccccagacctttggcctgcttccctcttcaggatgggtcctgccactcccagccttatgtcaggacccctccaccctcatagctcatactccatcaccctggagcctctccctccctcttagaactcctctccttttggacagagcctatgctccctctgagttctgctggcgccgcatcctctccagcctggctctcaccctctctgcctccctccctcatgctcagccacccagcctctcacacctctgagccttgcactccccctcttcaaccccctcaccccctccaacacacacacaccccaaacattttcccacccactagctctgccgggataattcctactcaccatgggcttcggagggcccttccctgactctgcaccatctcagggcctcgttataaatctctgacatgagcacctgtgtctctcttaggccatcgatcgatcatggagctaaatctgcttccctgccaggctgggagccccagcagtcagggcggggtctggtctgctccttctcagtgctgtgggggtacagctgcctgggtgcacatcccagctctgtccaaccttggattccccctctcagttcttggaaaccaaggcatgccattcaccacagaccctcctagtcctcccagggggtgacaagcactgtaccttttgcatcctgcttagctgtatgcttttttcccagagggagagaaagactctgggagttctaatttaactaaattcaatttaactgtagccatgtgctgggtcttttgacatatgtcatctggtgaaatcctcattacagccctgcaaagcagaagttatcaacccattctacagataagaaaagcagagcacagaaaggctgagttttcaagagatcacacagatgttaggcagtggggctggactttcaagctctgggttttgtgattcagaagtcaggactttcttcctttttcatagcttggttgattcctttctaagcacttatgaatcacctgaggtgggctatggcagagctgtgaagatggactcaccacacagagagcgtgcagggctcagtggcttgtgcagtgagcggtgaaatagaacagcacctttcctacggttccagcaagctgggttctgctccctgagaggaattgcataatcattaaggtgttccgagtggagaaggaaaggaggctgttacaggctagattgtgtctcctccatccccattcatatgttgaaaccctaactcccagaacctcagaatgagactatatttggagacagggccttaaaggggtaattaagttaaaataaggctggcagggtagaccctaatccaatctgactggtgttcttataagaagaggtgattagaacccacagagagatgttaagatgcaggaataagaatgtgaggacacaaggagaaggcagtcaccttcaagccgaggagacgggcctcagaaggagccagccctgctaacaccgtgttattggacttcggacctccatagctatgagaaacacatactgtttaagccactcggtctgtagcattttgctatggcagccaggaatcctcctgtaggatctgacatgccttggtctgagtgcttgcagctcatgtttgtagtcaggtcatcttttggagatttgctgaaaaagcagtttttcaagatgaactccatgtgtgggaggccttctagggcacctccgcatccggcgtccctggtgagctcatgcgtgagtccttttgtggctcttggcagggctgcctccatccctaactccggtggctgcttggttgccatggcgacagcttcctccatcctgagagcaggctcctgccgagacatctggatgagcctcactgtgctcggtgaaatcggtggagcccaaggctgcagccctctttctcatggatccctgctctgcacccagcatcccccttgctgaggacacaagccagtccagccagcctggcatcagggtccttggggtgcccaccccatggggaaagctgcagatggtgtggaaatgcgtttactccagaacagtgggaggaacgctgactggaagttagggagccaggatttcagtccttactctgccagggctctgcatgtggccctgggcaaatcactgcccaccctgggcctccctctcctcctgggtggggttggcacatgggcagttcctgcctgcaggggtggactatttgactggtctgtgctaggcatggctctctggggttattaccatgtctcaaagttcaattacttgctgtggtttttagattagcttgtcgtgggatctccactggggcccatcatgaggtgaaccagtatttgcacactgcttttggcgtttgtggagtgctttgcatacttgcttctctcttgagctccttgccagaaccaaccagtgatgcttcttcatttcacatacctggctatatttccagtcagcagagatttgccctgtagaggagcgagcagcccttggttcctgtgcccaccttacaggagaggacgaccggggggcagactggtgcagcttagctgccatggctcctggaggtgcagccacctcctccagcctcacgtggggctggtgtggctgagctcgcgtggctgggctccaggagagcaggctgtgcctctggtagcaggagatgaaggagtttcttttttttttttttacaggtgaaataaattttaatgataaaattattttagtaataaaaatcttaataataaaatgtatttaactaaatatacccgaaatattatcatttcaacattggcactagctacatttcaagtgctttttttgttatttaagttctagggtacatgtgcacaatgtgcatgtttgttacataggtatacatgtgccatgttggtgtgctgcacccatcaactcgtcatttacattaggtatttctcctaatgctatccctcctccattcccccactccatgacaggtcctggtgtgtgatgttccccgccctgtgtccgagtgttctcattgttcacttcccacctatgagtgagaacatgtggagtttgattttctgtccttggaatagtttgctcagaattatggtttccagcttcatccatgtccctacaaaggacatgaactcatccttttttatggctgcatagtattcaatggtgtatatgtgccacattttcttaatccagtctatcattgatggacatttgggttggttccaagtctttgctattgtgaatagcgccgcaataaacatacatgtgcatgtgtctttatagtagcatgatttataatcctttgggtatatacccagtaatgggattgctgggtcaaatggtatttctagttctagatccttgaggaatcaccacactgttttccacaatggttgaactagtttatagtcccaccaacagtgtaaaagcgttcctatttctccacatcctctccagcacctgttgtttcctgactttttaatgatcgccattgtagctggtttgagatggtatctcattgtggttttgatttgcatttctctgatgaccagtgatgatagcatcttttcatgtgtctgttggctgcataaatgtcttcttttgaaaagtatctcttcatatcctttgcccactttttgatggtgttgtttgattttttttttgtaaatttgtttaagttctttgtagattctggatattagccctttctcagatgagtagattgcaaaaattttctcccattccgtaggttgcctgtttgctctgatggccattttttttttttttttgctgtgcagaagctctttagtttaattagatcccatttgtctattttggcttttgttgccattgcttttggtgttttagtcatgaagtccttgcccatgcctatgtcctgaatggtattgtctggatttttttctatggtttttctggttttaggtctaacatttaagtctttactccatcttgaattaatttttgtataaggtgtaagggagggatccagtttcagctttctacatatggctagctggttttcccagcaccatttattaaatagggaatcctttccccatttcttgtttttgtcaggtttgtcaaagatcagatggttgtagatatgtggtgttatttctgaggcctctgttctgttccattggtctatatctctgttttggtaccagtaccatgctgttttggttactgtagctttgtagcatagtttgaagtcaggtagcgtgatgcctccagctttgttcttttgcttaggattgtcttgggaatgcgggctcttttttggttccatatgaattttaaagtagttttttccagttctgtgaagaaagtcattagtagcttgatggggagatgaaggagtttctataaaaacctctgcatgcccgaggactatacgggaggcctgtgtggatcacacctcctgtgtcctcggaagggatggctgcagacttcactcttgggtggaagaaaccccgctttgctgactcccccaggtgcaggttctgagctcacaggggtggtctgaacagctgggggcacccagcacccctacccccacccaccagggtgaggagctccttgtactgtggatgggggaccgggataggcccacctgtccctccagggctgcacttgctccatctgacattgaacctgggcctgtgtgcagtaaagaagggaggctgtgtgacccaagcaagactgcatcgcctcctgtaggcctggggctgtgggcggcagggcaaatccactgtgcgtggggctttctgtgcacatagccatcctttgtttagctagcacctctggctggttttctgttacaacagcagagttgagtccttgcagtttcgatagaaatcctacgtctggctaggcctgaaatattgactctctgactctttgcagaaaacacttgccaacacctgtgaatgttggcactggaaggaaaagggggtccatttcaagacatggggggctgaagccagacaactgccaggtccccgggcccctccagggacctgacagcccctccttgcccagcacctcgctgtccctgtctcatgcccatgactgcagctgtgactttctcctcctgctccctgagcctcagatgacacagagtccagagaggctgagactgacccgaggtgccacagcagatgaaagggggatttgaggctgggacccagggtcccacctgacagcctttccctgcccagcacctcgctgtccctgtctcatgcccatgactgcagctgtgactttctcctcctgctccctgagcctcagaagacacagagtccagagaggctgagactggcccgaggtgccacagcagatgaaagggggatttgaggctgggacccagggtcccacctgacagcccctccctgcccagcacctcactgtccctgtctcatgccggtggctgcagctgtgactttctcctcctgctccctgagcctcagatgacgcagagtccagagaggctgagaggctagcccgaggctccacagcagatgaaaggtggatttgaggctggaacccagggtccctggcagccaggcagaacaggccgcagaccttctcagcagctcacctacagagcgcccactctgagcctggtcctgttctcagcgcttcacgtggactaccttaccccatcctcatctgggagctgcagagtgcaattgcccttctgaagttggggaaaccgaggcacagagaggttcagaaacttgcccaaagacacatagctagcaagcggcagagctggagccacccccagatggtcagggggcaaggattgcactctggagccacggagggcgtatggaagactctggagcccaactgagtcccaggcctggtctgacccttctctccttgtccctgagcaattggcttctcctcgccgagcctctgtttccaatgtagaaggggcacacctcactcatggaaggcagaggggattggatgagctacagatgcgaagctagtcccacccagccagccggccgcacgtgttagctgcgacaggtactaggtgcttggctggggtttgggggatataaacagtagaacaactttgcctagtcaggtgaatgacacaagcaggtcagccagtatttgcaacctaaaaagatctcgctcatgggccttggagcaggtgcagtggagaccagaggaagcagtgtaggaacaagttcttcctgatggacttcactggacccgcagcacgtgtgacgtgcttcccagaccacagtgctcttttttttttttttttttttttccaggcagagtctcactctgtcacccaggccggaatacgtggcacgatctcagctcactgcaacctctgcctcccaggttcaagtgattctcctgcctcagcctcctgagtagctgggattacaggtgcccactaccacgcccagctaattttttttgtatttttagtagagtcagggtttcatcatgttggctaggctggtctcaaactcctgacctcaagtgatcctcctgcctcagcctcccaaagtgctgggattacaggcgtgaaccaccatgcccggccagcacagcactcttgcgtttgtaccgcagcagcaatactttcaacacgctgcagggggacggctcctttgaatggcctttgtcagtgacgatccactcctaaaaaacccctcacaccctggggcctccttgaccatgcctcttttttttttttttttgtgctttctgtggctttcttttttcttttttttattatactttaagttttagggtacatgtgcacattgtacaggttagttacatatgtatacatgtgccatgctggtgtgctgcacccactaactcgtcatctagcattaggtctatctcccaatgctatccctccccgggagttgaacaatgagatcacatggacacaggaaggggaatatcacactctggggactgttgtgaccatgcctcttatctcaactttaacagctgcttccccttgagatggtttgcaacctcctgccccctgagccctgatcagaggggaccacaatgggctgattcattcattcagtcagcaactacttactaaacacctactgtgtgccaggcactgaaggtgcagcagcaatcctgacaggcaggtccctgccctcgctggggacagatagcaacaaacaggccaataatgtaataagcagtgattaatgcttgcaaaaagagaaagcagaaaatctataattaaacaaggtaaaataaatgtttattttgaggggcagaggagatttacaggaggttactttgttaaggagcattgaggcagggatctgaagggcatgagtgtgagggccctgcagtgggaatgtgcccaacacgacctgggttgattggacacctcctccgtgcgcacaacctgcagcttcttctaaggggcaatggggcctggctgtggggggctgtgggcggaggaacccaagctggattcgcaatcaygcacccgggctcaggtatcagctctgctgctcactgactgtggaaccctctgagcctgtctctggacctaaggaaagcaaagtggagcaccagctgccttctgcaccctgttagtgtgtgttggggtgggggtggggggcaaatgaccgtgaacagagtttctaaattggaagggctgttcccagggaaaggttcagagtcgtctccccatctaatatttgcccacggatcagtcatgcagcgaacatgtcccaggcaccagctctgggcaggtcgtgtgtgaggggtgtgggggcacggaggtgaatcaggaggtggagctggccctggagccgactgcagggcgggacagatggtcacatacacagcacaatggtcctgaggatgagcagacgggtaaggccttgccttggtgttcaggcagagactaggaatgctccggcaacttcgaactgctggatatggctggcgggtatggagggagaagcggccaaggtgagactgaggaagagcaggcaggggcagcacgctgggcaagtccgacctcctcggtggggtgggagaggccaacacgcagggtggtggggagtcccgcagctgtttgaggaagttcactctagctgagcttgggcacggacccccatggggacagagatgctgatggcctgggctgaggggcatagcgcaaggggttcagatggaggaaccccaggacctgaaggctggctggacaagggaagggcttataatgaggaataagccccctgatgcctggcttggtgccccaaggctggggatgaggaaatgggagttctagagaaggagcggtgtgggggctgttggtgtggggggctgtcggtgtgggggggctgtcggtgttgggggctgtcggtgttgggggctgtcggtgtgggggggctgtcggtgtggggcggctgtcggtgttgggggctgtcggtgttgggggctctcggcgtggaggcgctgttgtcttaacgccctttcgttctaaccgcccttctgttttcaccgccctgggctctaacacccctatctctccaaccccctctggatccccccccccactcttttcctcgcccccctaggcaacgccttcttaatgtgcccaaagcccccggcccccctgcttggttccccacctctttttatgagtcatatcttattctcttctctccaaccctctctttcttttttttctctgcctccctccctcctcctccctcctactcctctctcttcttctctctccttttctcttttcctttcttttccctcttttcctttctttcttactcctctctttcccctctccctccttttttttcccttcccctttctccctctatctcttttttttccttctttctttttctcttccctcttcctttcttcttctttttctcctttttcccctctttntttctatctctttctctctctcttattatttctttctctccttcctcctccctttctttctttcctccatctctccttctcctatccctctctctcttctccatcttcttctcattcttggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacnnnnnnncnggcgtaggtggtagatagttcttaggtcagtcgcatgtactagcggtggtgtctagctggtcgtggttgtgtcggagggaggcgtcgtagatagtgttatgtatacgaggtcgaggtcgtcggtgctggtaggtgcgatgttcgtgctcgttgtgctagggatcaagatgtattagtggtctactggtgggcggtagtactagaggtgtcgcacggatgtgggttcgtggtggatttgtgagaatgagactggtgtgtggcgggagttggttaagttgtggggtgtgtaatggataaggcgtctggctgtaggagtttgtgtgtcggttgcgtgggggcgtgtctggttgagggggcgggctgtgcgtgtgtcaggggtagctgtggtttgtgtgggggtcagttctgtgtggggtgggttgtcttgttttgtgggtgatctgtgcggtaatagggggtgtgttgggttttggtggactgttggagtggaggggcgtggttggtgtgtgtgggggcttgagtgtatgggggggcgtttgctgttggggatctgtcggtttgtgggactgttgatgctgtggagtgtacggtgtgttggtgcacttggtgttgggggggtgcctgtatgggggggttgttgggtttttggggtctgatgcgtgttgggggctgttggtgtggggggtcggtcggtgtgaagggctgtcagtttggggggtctgtcggtgtggggcggctgtcggtttgggggggccgtcggtgtggggggctgtcactgtgggggggctgttggtgtgtggggctgtcggtgtggggggctgtcggtgtggggggttgttggtgtgggggggctgttgggtgttgggggctgtcgggtgttgggggctgttggtgtgggggggctgtcggtgtggagggctgtcggtgtgggggggctgtcagtgtgggggctgttggttggggggctgtcagtgttggggggctgtcggtgttggggtgctgtcagtatgggaaggctgttgggtgttgggggctgtcggtgtggggggactgtcggtgttggggggctgtcggtggctgcccatagcactggcattgcgtctgctcttatttcccaactcccaggaagcaccttgggtgggttagtgtcctgtggctgctgtggcaaattgccataaccttcatggcttcaaaatacagatatattctcaccgtcctgggggccacaagttctcaatccaggtgttggcagggccgtgctctctctgaaggttctaggggaggaggcttccttgtctcctccagctccggggctccagatgttcctgtagccatcgctcccatctctgcctttgtctccacatggccttctcctctggtgtctcctcttctgtctcttagaatgtcacttgtcattgcatttagggcccacctgggtaatccagggtgatctcatctcaagatctttaactacatctgcaaagacccttttcccaaatgagatcacactccacagtctctggggatcaggatgttatggacacatgctatgctgatgtatttgtgggggccattattcaacccactgcagtggagaaaaatagtctgtgctcagttgaggcatatgggattgaagagcgattttaccttctctccttttcatcctctctgcataacttgctcctctctctggaattcctacctgtgtctgacaaacttttcttgcagggaaaatgactcttagatgctagagtgtgattggaagggaaacaaatcccaccagacagttggagtggaattagatgggaaaacagaagatgaataattcaagcagctttgccaggggaaaggggactcttaaatgagcctaattatgcttgttcccaagggaggccagtcctgaaatgaaccttgtgaagcagtttttcaagcaggatttaggcgggtatcatgaagctggggtggggccacctgcggttagtcctggaaaggaaagggcccaggccctcattttgcagatggggaaagcaaggcttgcagaatggaaatgatgttccagtaacagccaagaccagacgcccagccgggggccccgagtgacagccaagaccagacgcccagccgggggccctcagcttctgacctggtgctggtcctgcctaagttaaagccaccgagtgtggtcactgggccaaatgcttcaaagtctacacacgggggagggcatggtgtggggcagaaagctgtgttcacggtagctgagtttgggcataattctcgtgctccggaggcttctcccacagctacttcccttgtcaggagtccatcttcagggccctgtgttatgatggattgagggagcaccaacaggggccaccccacttggtaattaattccttttgtaaagggctgggctgcaggcccgggtaggtcctatggccacctgagccaaggtcttggagaggcggtcgcacggaccaggtgaccatggacccagcccggtgcccacaccgtgaagggtgctgtgctgcccagaggttctgggcacggctctgggccctgggattcagaggctcgggactgcttgcctccccacggagatcttaccgtggggaccctgttgtggagggcctgccgcattggccgtgacagtgatttttctccttctgcagggtgatgtggatgcagggcctctgtgtctcacatggttgtttcacagcagccaccgtatccgaagacagagagaaagagcaggagagattcccctgtgatggctcccccatgggtcctgacccaggtttgggagactttatctccccaaagccaggccctcgatccctttgcctttggggactctgtgtccccagcctatacatggggtgctcgattgagtgtccgtgcccctcagccggccctgggttgccgtggctcgggtgtgtttgccacagggctacagacctaatggagccctgcatacttttattaattaattaattaatttatttatttatttttgagatggagtcttgctctgtcacccagtctggagtgcagtggcgcgatctcagctcactgcaacctctgcctccccagtcaagcgattctcctgcctcagcctcccaagtagctgggatttcaggcatgtgccaccacgcctggctaatttttttttattttttatttttagtagagacggggttttggtatgttggtcaggctggtctcgaactcctgagctcaggtgacctgctcgccttgacctcccaaagtgctgggattacaggtgtgagctctggtgcctggcctcctacatactttgaaaagttctgaaacatccccaggtgggaaaggaaagagcgtttgggtggacactgaacctgtcagggggctcacgttttgcagtggtagcaaacaacctgaaggtctccaaggcctcaaacacccacttcacacctcccactcacgctgcaagtgtggccgtcacaggctggctttggtgcgatctcattgcggagggaacagtgtcacagcagaccacgtgctggctctcaaaacttcagcctggaggtgacatgcctcacttgtctattcattggcagaagcaaccagcctctggccactctgggttctgaaaggccgaaggagaggtctccccttgggagagaacctggaatgttcagagaaccccctgggtgtcagcatttgacttcatcccaccgagtcctcctgcacccactgcgccccatgagttataaccccaggaaatacaacgagaatgagtgtcctgatgacgcaaacagaactcctgtgggactcacgttcctgggcgcttctgttgccaccaagcctcctgtggcttttgtgtgtctcagggtgtgcagagagaatgtagcccctctctgccctgcctctctactgcacattatccatgggccgtcgcactaatgtgcgtgacatttcatcagtggccgcactttttctctggatcccactttagtgaagttcagtaaggacctgagagctgctccgagcgggcagcgtgtcctggaaagccatgggaggcattcggcctgggagggctgggctgccaggtggacgctggagaaatcagcatggcaggaggagggctcttttcttgcttggccttcagtatcatttttcattatttaatcgctgcttcttttcatactggaaaactgtagtttcctgggaaaccagccaggcagtgatgccttaactcatttttccctctctttctatttttatgtgattctcagtttgtggttaatgacgtgcttccgggaagcaagatttgagcacgagcacagagacccttttaggtgctttctgactgcacagatcagccatttttttccctggcattttataaaccctcgggtttagagaggtcagaaaatactgcttgcttgctttttttttgacagagtcttgccctgtcaccaggctggaatataatggtgcgatctcggctaactgcaacatccacctcccaggttcacgtgattcccctgcctcagcctcctgattagctgggactacaggcatacgccactactaatttttttttagtagagatggggttttgcaatgttggtcaggcttgtctccaactaatttctttttgtattttagtagagacggggttttactgtgttggccagaatggtctcgatctcctgacctcgtgatccgcccgcctcggcctcccaaagtgctgggattacaaatactgctttcttactgagagaggcagcagcttgggtggaggaagagggagggcagatggatttcagagtttcagtcagtattttccagatagaaaataataaaaatggaaactgacattcattgagctctgcaacgcatcaggcagtgtgtgaagggctttgcctctgatgatcttcacagcgaagtgataaggctatactgctgactcattttacagatgaggaagctgaggcatggatagctgggaactcactcagggtcacacagccaggaggtgacagagctgggattcaaaccccagaccttccaactccagggctcacatgcacctgaagagtcagagggaacagaccgtgcaaagccccatgcagggccggggagaccggagcctgaggtcattgctgtgagaggggagagggcctttctgtcagggtgatgggtgggatctgaagctggaaagccggagagaagctcgggggtgccagtccttgcatgctccaagagattctgatctgctgctgggcagcagggaagggataggaaaagagcaggtgctggggccggggaggttgtgaggctgaatccagccttaccaatcacacctgggccaccccggacgagttgctcatcttctctgtgctctagtgtaacagtcagggatactgcctttgcaagcagcaaagttcttgactctaaacaaaaaatggacatttattggctcccacatttagcagtctggggggaatggcttcaggtgcagcttgatccaggattccaatggcatcaccagatctttctctgcactctttccactgtctgccccaccctcaagctccagggtttctccttgtcctggcagcagatagaagctccttctttctacaccacccagcacagcaggggtgaccaagctgcatcccagccttttcagtattccaagtcctggggtctggagagcacagatcagagccgaggtggggccgacccagaaacccagaagaacctttgcccaaaggagaatctccatactgtggtcagaagaaggggagcaggtgccgggggcaggaaggggagctcccagatgcccacacactcaattccagacacaatttgaaaagggaccgggaagacttgaaatcattcaccaatagcatccactgcccagcatgcagtcggcacaatataaatgctctttttcttctgcttggcaaccaacaggggaaggaaggcagaggagaggggctccacaggtcagtgctatgtgtggctggaactgatgcaccctcactggggtggggtgcaggtgaggggggccgcaggtcaggagggaagtggtgggtccagcttcatgggaacaggggacaggcacctagaaggggtagccagcaggcagttcaccatgcaggtctgcagctcagggggacgcattttgagggtctcaggaacagagaggcagctcagacgcgagggtggacggttttgcctcgggagaggtgtcagaaacctccgggaaggcggagcattgagggcgcccttcactgccccttccttgaccgcactgcccccagacatcgtggacatcaagccggccaacatggaggaccttacggaggtgatcacagcatctgagttccatccgcaccactgcaacctcttcgtctacagcagcagcaagggctccctgcggctctgcgacatgcgggcagctgccctgtgtgacaagcattccaagcgtaagtgccggtgcctgggggtggggggctgtgcattgggcaggcgggcgggtgggatgtcctgtcctggtgcagctgctgcaggggtggtgggtgtgggatgagcatgtcctggactggccaaggtgggtgggcccattgctgcttcagcgctggctggtcggggagcctggtcttgccacctgctgccccccagagtccctgcacagccaggaacagcccagcttttagggttcagggctctagggcgagggtcagggcatccagaaggtaaggaggcaccaaacttaaatgtcacctcctgtaggaaagcttcccggcctcctactttagatctggatcctcctctgtggccctgcagcccccaaagcttccctcttccaaaactagccacacccagcaaacttgctgtgggctgcccatcttctccggctccttggagcagggtctgtaacatgtcccctcatcctagaacccaccacaggcctgctgtgggggtcccacaggggtatccgtggaggtctggggctgaacgagttccaggggacattttgtgacacagcagcccccagcataacagctcacactgaacactcactctgagcccagcccttaccaagggctttacctgtgaactcatcgaatcttcacacaagccccaggagatgctgttcagaagaggaaacagaagctccagttattggcccaaggtcacccaaattcaatcgtggcagagctgggctgcctggcactgggttgtggccatgggctgcacactgccacagccatcccagaggcccctgaaggcgagggatcaggccacggcatcctccttcccagacagccctgcagggctagcaaggggccaggcaggaggtggggtgtcccccaggcagagggcagggactttgcttatctggttcccctcactgtctgtccccagcacctaccacagtgtctggaaataaacacttgttgaataaaccccagaggaccctgactgaagcctgggtgccggcggcagagggctgtttgggagcgatggggccacactgttcccttagggtgtctggttttgctgggcagtcagagacagggtgcacaccagccccaggactcaggctcagggcccaccaaaacctccagcttctgcctccttccttggccaagttctggcccccacatccaccccctcatctcctctgggtttgtccacgctccgtgtgtcacgctcagccatccaggcattcagtaggcgcctattcctggcatgggtgtgcagcagaggcccaccgtcgggagctggcgtctgcgggggagacagacagtgaacacacacctgacgcatgtcagggcatggtgagcaatggcaccaacacccacaaagtcaggtacagggacagagggggtcagggagggcctccctgaagagaccacagtggaacggacaccctgagggggtgggtggtgccgatctctggggaagaatgttcgagaaggagggcactgcagtggccgtgactccacacattgcctgtaggtcaagggagggactgggttttgttctggctgagctgagaatgagaagctgtgggttgtcacgagattgggacttgaggaggaagaggagacgccgcagagggtccccgggggagcaggcgtcagaggtcgctgcagggtctgcaggaagaaatggtgaccccactgagggggttgttgggaggtggggcagggctcacctggctgccccctctggatgttgcttggaggtcgagctttgccttgtggggcgtgactctgtggttggtttgagctgtgggtgaaggtggggccgtggcctggaacaacagggggtgccagggaccagcaggcaggtttcagacccgcgagcctgaggcccgtattgggttccaaggaaatgggtcgagtaggcagccagatatccaggcagtaatgccacctggagataagtgtggagttagtggccgtggactgatgggaggctggagaccagctgagattcccggggtgcagggggctcggggctgcgtcctgtgcgccacaacaaaggaccacacacttggtggctgaaaacagggcatatgtatagtctcagtccgcagtcccacagccagctcactggccataatgaaggtgccacgggctggtaccttcgggaggctctggggagcaccgttttccctgcctcttctggcttctagaggcgctgcgttccctggcacgtggcccctcccccatcctcccagcccacagcggccaagcctctctcacgtctctgaccctcctgtctgtctttcgcgtataaaggcccttgtgatcgccttgttcctgccacccctaatgatccaggatgacttcctcatctcaaggttggctgagttgcgtccttgatttcatctttggccttattttgactttgccgtgtagcataacatagtcacaggtgctagggatcaggacgcagccgtctctgggggccgttccaccgcctactacaggacactgcccccccacctacgcctcccgcccattcactcaggccacatgtgcctccagctgtgcctgtgtacagccaggtctgggtccgaatggaatgggagaatattggggaagagagggggttcttcctgctcctggtccgctgctgaccccgtgtgagccctgtgggaaggaggatggggcttcattggcaccctgttgtttataggggtggaaatccagaaacatcctcaatatcccaatcttaaatgctaagattattcaactcttctgaaatgagtgaggcagctttttgtttctttttctggaaccgttggtgtaatatatctgctccttgaagtattaggaaaacttgtgtgcaaatctattcggcgtccttttgagggagagaaggaaaattttaagcaaccagtttaaaatgccgagaaaatgatgtcgacagtgatgacatccatgtactagaatattgttcggtcatgtcagctattgttttagaagaatttttaatgccacagggaagtgattatgttgaaagatttaggggataacaaaataaggctatgaccaccagaaaagtggaaacagagaaaactctggaaagaaatacactaagttgtccctgagtggtgagctataagaaactgatggaatattttctttttatttttctgtgtatttttactttcatgatggatttgaactttgaatcagaaaacacatgtgtgtgcaggaagggtggaatttagagagcacagagacgggcataggacagttttgtgcacccctcaatgtttgtgtgacatttttcagtgggctagagggggcagtcagaccccctttggctcccgaagcctgtgctatccggtttcccagggcagaagcagcggtcacatataccttgcacatttcgtgagtccaagcacagacacatccatttcagccacgtaatttcatatgcctgatgatgtatgagccttgtgtgaatcagccaatgaactcaacagtgcgagaaatatgagtgttttcgatgactcaattcaatttcattcttcacccccagctcccagggctgcagtgaacgttaacttttagggcctcggcagattccactcctctccggattgcactgaggttccagaaggctctggcaggccgaggtgcggagagagggacatggtgtcttccaggccactgaggacttttgattgtgcctgggcgaccttggggccaggtccttgctgacaaatccccaagggcctgttggctgtgctcagtgactcacctgggaggttggcaacatgctgacctctggcggctcttgcccgcaccagtgatcccaagtctgtagacagggtggggctgcctgtgatgtactaaggttttatgtatgctttttgcacacacagtcataggtgaagttggtttataattttcttttcttatactctctttgcccaattttaagattattcaactctcctaaaatgaactgggcagctttttctttctttttctggagcagttgatgtaatatatctgctccttgaaatcttaggaaaacttgtgtgcaaatctatttggtgtccttttgagggagagaagaatttttttctttttttttagacagaatttggctccatcaccctgacacgatcttggctcactgcagcctccacctcctgggttcaagggattctctcacctcagcctctggagtagctgggattacaggtgtgtgccaccatgcctggctaattttttttgtatttttagtagagacagggttttgctgtgttggccaggctggtctcgaactcctggcctccagtgctgggattataggcatgagccaccataccaggccaagagaagaaaaattttaaacaaccagcttaatttcttttttgattattgatttatgcaaattttatttaaaatttttctagaatatttttcattatcaaactcaatatcctgtacggaaactttctctttcgtgccagcaaaaagattgcgcagcagtttttagcatgcttttacagcttccatttctcctcatttaatttgccgcacattggggcgagtggctgaagtgtctgtccttctgcatgccttgtccgggctgtgtttttgctctgtggctctcatgggaaatcaagcacgcagccgctttccaattagcaggtggcggctgtttttaatctgaacacaaatggctgttatttgagctaattttttaaaagggatttggggattaggattagttcttcacctcccacttccatccatgtatccaattactgctcaaaactcagggagtggctgatagtgacaccccagagatgtgctcacagcatcattttcctaacagaatcagaccgcgaatgaagagcgtcctggatgcaggaaggtgagcatttctgccattggacagatgaggagaccgagggtccgagaagctctgtgacttccctggacccctcctctatcagggcagagctgggggcacacaggccactctgttccctctgcagcttcccacctttctccgaagcactggaaatccgcgtcttgtgactgcttggcagtgtgacagggtaaccgtggaaacaaccgtacatgagtcctcaaaatagacagttacttctgcttatgacagcacagagcctgcggaaagaaagggccgtgcccggaacactgggatttcttagaattgggggtgatgatggctgagaccctgatttcttagaatcggggccgatgatggccaagacccttcataggcccccaacgacgtgcctggcccggtgttgggtgaaataataaccgcagctcattctttgcacacctagtgcaaacacatgctgttttcagtttatatctcattaaatactcagcacctatgatgggtgtgttatagtattcatcctacagatgaggaagttgaatacacttgaccctggaatgatgtgggagttaggggttcagacagtgcctgaatgcagttgaaagtacatgcttaacttttgactccctcaaaacttcactcctaatagtcggctgttggtggggagcattaccagtaacatcaacagctgatgaacatttgaagagactgatgtctacgtggattttatgcatttgtgacatacctaactttttcttttcttatatatatatgtatgtatatatatgtatgtgtgtgtgtgtgtgtgtgtgtgtgtgtgtgtgtgtgtgtgtgtgtatcgtggcagatctccaaaatgttttcagtatatttactgggaaaatcgcacatcaggggacctgcaggttcaagcctgtgttgtgtgagggtcaaggggcaactgtatacgcacttctcattgctgcgggaacaattgccatgagcttagtggctcaaaacaacacaaaagactgttgtacattctggaactcgggctaaaatgaaggcgtcagctggcctgtgttatctctggaggctgcagggtggggctgttttctgatcttttccagctactagaggcttctagaggctgcctgcactcctcagctcatcattccaacctctgcttccatcatcccatctctttcttccatctttgaccttccttcttcctcctatacagacccttgtgatgacattgggcccatccagattatccagggtcacctccgtacctcaaggtccttcacttaatgacatctgcaaagccccttttgccatgtgagataacaggtgcacagattctagggtggaggatgtagatagacgcctttaggggtcattgttctctgctacattgaagcacagagagattaagacatttgcccaaggtcacacagctaagtagagccaagatagagcctcagagagtctcatgccttcaacctgcactctttttccctttctcatcacagaagccttgagaactaaaactcttacaggaattgtgggtgagctgggttttttttttttttttttttgaaatggagtctcattctattgcccaggctggagtgcaatggcatgatcttggctcactgcaacctccacctcctgggttcaagcaattctcctgcctcagcctcctcagtagatgggattgcaggtgccggccaccacacccgactttttgtatttttagtagagacaaggttttgccatgttggccaggctggtctcaaactcctgacgtcaggtgatctgtccacttcggcctcccaaagtgctgggattacaggcgtgagccaccgccccccacctgagtgggttcatttaaaagggctgtgtgggctcaactcccagggctcactctagtaatggatctgctgcagaaagaccagcctcagcccagaccctgcagctctccaggtgcaggaccagggcaaagctccccaacagtgggaacagcccatgtgagggccctgtggctggatacagaggctggagagaggaggtgaggtcagagaggtgtgtggctggaggaggcctcacaggccagagcaagagcttcgggtttcctctgaaagggacaggaaccacaaggccgtctgagtagggagtgggccagcctggccatgtgttcacaggatccctctggctgcccagtggggaataagctgaggggtgaggtagaagtgggggctggctgggggctagcgtgttcacgcaggtgagagacgctggtgcgcagagtgagtggccggtgcaggtggggaaggtggtgggccctgccccccatggcaggtggggcctgctggatttgctgctggattggatggagagggagggagggtcggggatggcttcaggctctgtacagagaccaggctcccgtctgtgtggagctttgtggagggaccacaggaaggggactagtaaataagtaggtgcattggagtgggaaggtttccatgaagggcatcaggagccctctgatgcattgcacctcaggggtcaggagaggtgtccctggatgagaaagagcctgggaatgggggcgggggtgccccaccccacagtagtgtctgagaggaacctggctgacgtcaggacccgtgtggctggaacaggtggaggggtgggctcttgtgtgcaaagatgggttaaggggagagcgtgccgggcctgggggccacagctgggcttgagttttttgtttgatggggctcgatcacatggtgctgggcagggagtgacaactgtcatctcttcatcagtgagcattcattgtgcccacctgtgccattgtaggaactccctgctccctccccactgtgtacagcctcagggatagggaaagaggccgaacaggaggcccctccccaaacagtggcatcacctggaacccaggagcaagccagtgtccaggggtgctcggggatccataacccccatcgtccttcttctgcttctcggaccctccaagctcctcctggagcgcggcctgaggtgaggccagctggcccaggccactccaccctcgtggatggccttgtatcccatcagatcacaggagggacccgtggccagggtagccaggggcgatcactgcccaggaccccataggcccctccccctccaggccacctcctcccatgtttcccacctccacccacctgcaggtccctcccaccctcatctgtttctctggaggcctcactgaactgcacttgtccctgaatgcatggctcaagcccatgcatttctactgtgtgaacccaatcggcctccaccccagccccagaggagtgtcgtccacacactgacaacgatgggggtggggcacagcaggtgctaccaagagggccacagagcaagtgccacgggcacacaggggccacccccaggctggctgaggcttctctgccaggcctggaaactgcagtggaaccacatcaggttggggaagaaagatgtcagtgaggggctggagcagaaacggcaggcagctgcacaagcttctctctgctcagggagcagggcggggccatgaggcagggtctgtgctaggcaggggcgccaagatgatcagtgactaatgaccaaggcaggacccctggcccaagggagcttacagccccatgaggggaccctgccctcctgccagtgccacaggagaaggtacccagcggcttgggggcagcagagggtaagcacaggatttctttagagagcatgtgaacaatatgcacagaaagcagggtgtgagcctggggttctagaggatgtataggagttggtaagaaagaccagctaaaaatgggaaagggatctcagggagagaaaatgatgtgaaaaataactccaggaatccctccgtagttttattgagcagctactgcaagccccacactgggccatcgcctccaggcatgacctctgaagatcccatcgtgctccttctccagatgagggaagctgggctcagagaggaccaggcccttgcctgaggtcacacagagcacgatgcattctgctctctgcccagcacatggcggggcaggggctacgggaggcagggggagatgcctagggctcaacattgaggagacacccacccccagagcctttgagtggatgcagtgccctaggggcccagcaggaagggggctggagtgtgcggcagaggggaacttgcgtgtgtgaggcagcagcagcaggctggttgggacccattgagaaggagctggtcagctacaggtaggagcctaaaccttgatcctgtgggcactggggagccaaggagggcttcaagcagaaggaggtgccgtcggctctgtgtgaggctggaaaggaaggtgggcacctgctctcaggcttcacctcctgtttcctttgattcacaggtccgtcagagccgccccacaccttggttgggctccccaggtcccctccaccttggcctgagccagggatgggggtggggaaggctctctgtggccacaacaccgtgcaacacccagggaggaagaggatgtggttgtcagggaaccggctcgagccatgagagccctaattatgcaaaatcattaaaaagaaacaggatggagatttcggctgattgagagctctccaacttttaaataattagggtgtgggggagtgggtggagatgggactgggcccatcacaagtgatgggggctttgagacatgtttctttaaacccatggcaggaataattactgtgaaatgtccttagcgattcagtcaattagaggggagaccccttatctcttgttagcgctctgaaggtggtgcagcggggtgtgtgtgtgtgtatgtgtgtgtgtttgcgtgcgtactcgggggcagcgattgatatggaatagtagattccactatcttttggaaaaaaatctattgtatcaaaaatttcatagaaacgtctaaaaagaaaaagaggaacttttaggaatggtttttcaggcttattaatatcccttccgatctatcgtttgcattcctgttccttgtgtccttaaaaaccctcagaaatgcccgagtacagccagcagtggggcgacaagggccagcgccgtttggctttgtttccactatcgattcaattgttctctcagcctttgatctttgcggtttttcctgctggaggctggtaggatacttggccactcgttctgccctgtgacacccagcaccggtgccccctgtgattctggctgaatccacccgcaagtcctgctgggaacctagaccagccctcaaggtctgggggagtctggtctgagagacccctgagcaggcgagggggtactaaggccactccggaagtcagcctgtgagaggccgccctggctcagttttggactcacctccgcctcacctccaagtgacagaaagcctagttcaggccagacagggttcccgccactcacctgggcaggtgctgggggccaccaggtcccgctgagattctcttccttgtttccatccatttagccaccgggggtccctcttctctcaagccctctccctgcttctcccaaatatgagtctctacccactgctaatggaaaggcagcacaacatcacccctttagggaatcaagcgttcgaagtgattttctttacggtaggcatcagagagaaaagggaactgtggttgatccttctttggcttaacaaagaaaaactacaaatatatatatatatatatatatatatatatatatatatatatatatatacacacacacatacagaaacaaagatgccaatttaattattattatcggggcacaaaacattatcatgtgagatgaccaattagaactcagaggttcagaagctcaaggtgactcccagggagattttactacccctaccgtacccatcctcctccacaggcagggaggggttggagggctcacaggagggagcattagggccagggcctcctccatttttatggttcggaaatcacatcgtatcaaggcagcataagtttgcacagccctggagggttgactcgtttggggcaaactcaagagggcctgatgaataagccagacatcactgactctgtgaatgccaacagtgacattgggataccacccaccaggggctggaccccttgctgagtgtctctgtctccaaaaacagctgcgcaaactgggcagtatttggctgcatttgacagagacagaaacagaaccctgacaggaaaaggcaagcatcttgaagtcacaaccaggaagtggcatagcgggtttcgaacccaagtctgcctgactcctggtcctgcaaggtctttcactataagcgatctaaacttgagccacttacttttctatataccaagggtcatcaaattctttctgcaaagggccagatagcccattttttatggtttcgtgggaacatatgttctctttgggcaaagtttgccaactttgccatttttatccaaaaagggttttcacataacaatgtgttggattccttgggggatgacttcttttcccaatcaatacttttttttacaaaaacctggctcttgggccaaatttttgtcccatggggcctgttatcttaccctcccccttgtgggttttaccttgttggaacaaaaaaaaccatccatctttgttatttttttgccccgttgacaccttttctatttatttaaattttaacaaaccgatccttttgggaattcatatttttggggctttttgttttaacgattgtcgctttaaaaatttaaattggtgccggtggcttccatcctatttatatcaatcatattaattttaagcaagctgatctttcggaggcccttctggtcccttggttgtggaaataactgttttctactataccaaagtatcttttacttgggcgggttggggttttttaaaatgttttgggaaaaatataaaatgagagagaaagagaaggagagagacgggaagaaagaagaaaggagagagaatagagagggaagaaggaagagggggggatagtggagtaggggggaggggaagagagagagaagaagggagaggagagaaaagaagagaggagagcgaggaagaggagaaagagaaagagagaggagggaaggaaggaggagaaagaagaggaaagggggagagaggagaagagaaagaagagaagagaggaagaggaggagaaagggaaagggaaaagaaagagagagagaaaagaaagaagagaagaagagaagaaagagaaaggagagagggagggaggggagaaagagaggagaaggaggaggaaaagaggaggaaggaagaagggagggggggggggggaggaagagagggaggaaggaagaaggaaaggggagagagaagatgggaggagggggaaaaaggaagaggatagaaaaaaaaaaaaaaaagggggggagagaggaagggagtgtgtgaagagggagaggagaagaagggggaagaggaaaggaaatgaggaagggaagaagaagagaaaagaggagaggagagaggggggaggaagaaggaaaagagaggagggaggagagagagagagagagggagaggaggaagagggagagagaggaggagaaaaaagaaaaaaaagggaggaaaggagggaggagaaaaaaggagttcgttaagaagggagggagggagggaagtaagtcagtagggctgcagcactcatcgtgcactgggctcactctgtacagcatctttgctgcctgcggccctgcctgtacccagggtggtgccaggcctggctgcaaccccctcctcctgccccttcctgaaaagcgcccttggctcctgttgtagggaggagcaaggccagggctcagggaggtgaaggcgttcaaggccacacgaggggctcggggattgagaccctggcagcctgttctctcccagctccctctgcgagcggcctcacccttccttcccatggtgctgattttctttctggtcctcccttcaatgtgcccttgtggaaagaggaaagctcgggccactgagaagggcgcccctgtgaacgatggagggagattgaggtcgcaggcaggcctggggactgggggcgtgtggaggggtgggttgggctcctggaaagggaggcgcctactgtcagttttgagtttcgttaaacacctgccagcctgatagcattctaattccaaaatatgacaccacttattagtggataaactaggctccctccctccttcctgatcttgacgccaggactggaaggagcgaccacctgccctcaggatacccggtgcagtgccatggctttgagggcagaggtgtggggttgaaggctgccactgctcaccagaggctgtggagaggctaaggacagggatatagatctcctgagcttcaggggtctgcttcagagacaggtgtttggaggccttgctctcaagagaggtgggggggaccttcctcctgctgcctgctctgatttggggggataccagaggcatgaacataaacgtgggagccttttcctgccctgtaagcagcttccctccccctcctctcacctcccctcacctccctgcatgcagtggcctgggatcaggatggtgacacctgggctctgcagccctggcccaccgggtgctggaacttccttcctccgtcgggaggggactccagcctcctgcctgcccctccctacctccttgactcagatttcatcctccctgtctctgaaccctcagtggctcccgtttccctaatggtgacttcctcacccctctccgtggcccatgagatcctgcgtgggtggccccttgcgtccctgttccccctgctcactcctttctctgcctgcacggcccttggcgtgtccctgcctcagggcccttgcgtgtgccgtcccttctgcccagcacacccctctccaggcatcgccaccaatctctccccaacttcactcagaccttggtcaaacagcacctcccacggaggccctgttcacctaaactaccagcccccagcacctatctgcctgctggctcttcctccccagcacttgctgctccctggcggtggagggcgccacttagttttattgtctgtatctgcaaggagaatgttggctgcacaggaacagtggctttatgctggtcactgcatcatcccagggcctggcacagagtaggtgctgtttgcattggttgactgagtgaataaatgaatgaatgaatgaatgagtccatcagggcatccagtgggccctgcagaggggagctggacaggagttgtatttctggagaggcagtggccaggtacagtgtccaccttggacaggagggagaatggggtgctgccatttcccatgaggggataaggggctgggacagacctgggaggcaggacacgagcccttgtggctgagaggggccagcagggaggggcctctcgggagcctcagggtgctgtgatcagctctgcttccctgtttctggggtagagaccagagcaggccagcaggcaaggctgccactcagccggtttccatggggacagctggacaggttgtcataggtttaggtatttccagattggctggtgaatggctgtcaactccaccaccctgttctcttcccatcgttccctgggtctctctgtggcccaggtcctagggagttagtgcctggcccaacaggggtcctagtccccacgctacttcagccccagggtcactgctaccagtgagacagataccagcagaaatgagcttagagaccttgtcccactttgggaacttctgcagctcaggaaggccaggttatgggggcagtggggaggggacactgtctggggagtcctcattgcccactctgtcccagtctataattgtccaggtgggcagcaaaccgttgcctttagggaccagataagcaacttcctgtgcagagcaggtgctccaagaaaaaaggagatggtcagtggatggcatacaggagactgtaccaactctgtggcagtcagatttgattttgttacaaaccccatggcaatgaaacaaaacccacctataagtagggctcagccatgcctgcccaggacaccatgaacagagataactactggcccaaggtcccagggccagttagtgccagagccacaagcagtgcccagtctggtagaggacattgtccagcacatttgagaatgtcaggacacctttgcaatctggcattcagcatcaccagtagggggcagtagagggcagcacatcaagtatagctttggcttcaaatcccgactctaccaccttcttccagcactgactccccaggcatgggttttagccagctgctcctccattttcttggctatgaatggggatagtaatggctatttctgcacagcacagaatcttaccaggcttgttccctggtaagtgtttagttctaggtttgaggaatgaatgaatgactgaataaacagagcatgggcccaggtgcaaaacagagtcatccgtcgtgccaaccccatgggcgggagcagcgcagtgacggccattgcttctctgtctccacagtctttgaagagcctgaggaccccagtaaccgctcattcttctcggaaatcatctcctccgtgtccgacgtgaagttcagccacagcggccgctacatgctcacccgggactaccttacagtcaaggtctgggacctgaacatggaggcaagacccatagagacctaccaggtgggcaccacagcaggagacccccaatcccgggtcttttttccctatgctgagatccccatggagggggccttcctagccaggcgtggctttcatatgcccggtatgtaggtgaagacacggaggctgaagaaatccagcaactcatccccacacgtagcttggcagagggcaggagtgctcagtcttgcctcacagggagctttggatgtccctgagcaaggcctgcagtccagggcccaggggcctcaattccagggaggaaagagatgtgggaacagagatgagcgtcaggctgggccccagtggagcatgtagacgatggcttnccctccccactccctgggagcctgcatcggagctgtaccattaaagagggttaagcgttgccctcccagggtgcatccgtaagcagtgcacgcagaggtgaactgagcacatgtttcctttccagatcctcagcacaggcctataagcctggagtctaggtgaagcctgggcccttcttggacagtattatttattatcttgctattatcatccattcagatatgttagggggtagacaacaaaactcatgtgatgttaaaataaaatgtggacttgaaagaaatgtgggatggcttcaaagctggttccagttaggtggggagatcagggaggcttcttggaggaggtggcatttgtgctgggccttgaaggatgggtaagatctggnccgtgcaaacatgggtgggaaaacaccccaccagaaaccccagccggttcctcaccgtgtcactcctcacacanggggtgccacgtgcttgacatctgtcacctccattctttactgtccggtgacaaggagccatcatcctatttggtggaaagggaactcgggcactgagaggtagggagatgagggaggtgtctctcggctgggcggtgacagattcagagcccaggctcagagccctacgctttcctttcctccgtgcgaagacctagtaggaaagcgtcctgggtggcgcaggcctcgctgggaactggtgcagagctcagagggtgggctgctctgatctgacctgggccccagaggaacagctcacgctcctggaagccacacacccacaaggaccgctatggggaccgcctgtttgtcagccacgtgtgtttacttgagttcttgcgactgccccagctcctccctcaggccccctcctcacactgcacaccccaggcaggactcagccctcctctgtccctgtgggaatggcagagaccccagactaggagagggacaggataagcccggctcaagcctgcaggagcaactctctggctccctctggaggcagctcagggaaatgaacatttccaaccccctccggccccttgtccggtcacttacctccatccctgaggtcagcaaaggcccatgaacccctgaaactacgtggaatgtgttctatgagcattttcagggggagggggacacagctttctgaaattctcatgaaggaccccacgaaggaggggaatgactgttctggagcgtgaggccgtaagctgggcacttggcatctggcccagctgacctattgatgctaaaataatagcaataactacataaaagcagnacaacaaccaacattttttggtggcttgtgatgtgctagaaattgtgtgtagcaggactgcgttcggctggtgtgcggtgcttaacaaatcggggctatttttctcccanaacagggagtctggagagggcagtccaggaaggtgggggggctccatgaccccggcagcgccacagtggttccttctgtgatccagtccaccatcatttgggcgttggttttccaccttcacatgtggtacctcatggtggcaaagtggctgccacacctccaagcactttgcctacattccaggcaggaaataggaaatgcactagatctcacccctggggtctgttgcgtgtttcactgtgattgtaaaatcttccccagaatgcttctcagaagacttcttacacctcattgtctagaatgggtccctggcagccaccatagtggctgggaagattaatggtttaacagcctctgtggttgaagaaggcaagggtgaaggggctggggacggttgttgaatgagccacactgaggtaagtccatantatgctttgcctcatctaagcctcccagctgtcctgggaggtggggccggtattctccccatcccattttattggaagacnattgagttaaatggcaaatctgtgacagagccagcaagcagnggaccttagattgacagtctttctccagagctcttcctcaccctgccactagagggcaggagatgcttagtgcacggcagagagaacagcatgcgcatttggacgcgtgcatcaccaaggctgctgggagctgagtaaggaggacacacaaaaatgcagagccgggggtgggacacgaacactaaaacctgctgccattcatcccccttgattttagttgttacacatactgagccagtaccctgtgcccaacacagtgctagaccctggacccagagagaaggaagaacctcggcctcaccctcgagggtcccagacagacaggaacacagtgaccctcagcttggtggggctgcagcccaggggcccaaggggaaggcaagggtcaccaggaggacacgggggctggggagggatgagcagggccccaggcgggagtcaggtagccttgtctaaccatcctgtgtgcagtgaatagcaggcccttcctcccaatcctgcttcccctagtgccactctgttttcctgtgtagtccttgccaccatattagtcagatggctcagagaaacagaaccaacaggatggggatagagactgagaaagaagggagatttgaaggaattagctcacgtggcatgttctaaatctgcagagcaggcgggcaggctggtgacccaggggagagtgactatgcgagcccacacgcatctgttagcggaatcccccggtcctcagggaggtctgtctttgttctatcagggccttcgactgattggacaaggccccccacatgatggagggcagcctgctttactcagtatatcagcttaaatgttaacctcccctacaaaatgtcttcacagaaacaccaagtatctgggtaccttggcccagccaagctgacacacgaaattgacgttcacaccacccttcacattttctgtgttcgttggagttgttaatttcaactcctgggagttgaaaccaggctccatggcacccagttaccttcccttccgcacccagagggcagagcccgtgccctgttgtcctggcagctccagcctcagcagagggctggcactcatgcggccctccgggtacagggcttgtaggaccggctgcagtcaggtggatgcaggtcctggggtgtcaccctctctcctgtggggtacgggaatcctggggaagggtcctggtcagcctcttagaggctgtgtgaccctgtgagcctcagtgcctcgtgctgtggatggatgagaaacctctgtgggttccctcttcccctttcttgatggccgccaccctgtgttctcggagatcattaccctcaaaaggcctgccctgcacttaatgccagaaccactgtgaggttcgccctcttatcactttaagtttgaagaaactgaggctcagagagatgaaatcacttgtccaagatcacacagctgggagggcagagccaggatctggaccccaggtggtcctggcccctgtgctgtgagcgttctgtttgtcacagtggactctgctccctggtgctactcccgtctctggccacagctcagaggtcagccgtgtgcctggtcgtgggcccccgataagatgagcagggctgtattgggctgtgtcacggtggaggtcagccgtgtgcctggtcgtgggccccccgataagatgagcagggctgtattgggctgtgtcacggtcggggtcagccgtgtgcctggtcatgggcctcccgatgagatgagcagggctgtgtcgggctgtgtcagagcattcagaccctcgctgagatgagcaggtctgcgctgggccatgtcagggcatgcagaccctcgctgctctttgagacccttcttgtggaagggccaggatggtcgggacgccccgtccactcacctcatcccttatcccaccaggtccatgactaccttcggagcaagctctgttccctgtacgagaacgactgcattttcgacaagtttgaatgtgcctggaacgggagcgacaggtaagccctgacctcagcccgcacctcacctcaccgtagggagggtttctgccctgcaggggtctgggctgggattccggtgacccgcagcatggggctactcagcctcaatgggtccaggtgtctgggtgaagcccacgctttccagagcaggtccaactctcagcgctcagattcaaggggcaggacatgaaattcttcatcttctgtcactgaacctcacagccacgttggcgcctgccctatgggcagtagtgggaacatgtttagttaattcagggtccccggtgatgtgctcccctctcccagcttgtcgggggcgagggctatagcccagcacccggtcaccatcatccatccacacctgtatgtcctgagacagccctgcacccctgtggctttgaccatcggtctactcacccctcctcccatcaccactactgtctccctctcttcctggtgacaccccactcgggcccgctgaggctcaggggcaccttggagctcctacaccctccagggcttgtcacaatccacaagtccagccgtctctcaaccccacctgcctggaaagtggcgccccagtgccagaagtgagttcctgtgtctccctagcctgggctcagcccagggcgggcatggacaagggggctgtggcaggggctcctgacctgacctctacccgtgggtccttacctctgtgtctcttctctgggattcttcctccattctggaggtgggaaaatccctcttctgccctcccaaatcacatcagctttgtgctcagggtcctgccaggcgtaagattctgaaatggacaagcctactctccatctgtgagtttcgatctcagaagctgagaggtggcctctcagtgtctcctacagctgcttcctcaaggacaggatgcctctttgtccagccgcccagattcagaagtgggtctccagatgatacacagtgtggagataaagactacactggctgctagatcagaataccacctgtcaggagcccatgtactgtcacctcctccccgcagtcctgtccagtgtggtgggcaaggagggtggagtgagagccagcagccctgacttgggcatcacctggtggggggctgtccaccctgtggatggcatcagtcaacatgacaggtctgggctctcccagacctctgagggtggctggcactgtggtggtcatgtgagagctgccgcactatgactctctgtggctctgggcatagggctgggaccatcagggttggtgtgtgggatgaggggagggctggacatggcagagacaggaccaggagggagccccctggaagcagggctggatccaagtggggggccaagtcattgtgtccagggaaggagatttctgagagagttgccaacatcctggagctcccccagcccgcaggggtctctcagcagagcccgagctcaggcaaggggctggcatggtgatcacagggggccactaaaggatgcttagaaaaccaggatggaggcccgacccggggctgggctggcacagctgggtcagcaggacacaggaccttctctctaggccctgcccccagatagatcccagacacccccagcagacagggctcctcccatgctgctgagctgcatttggggttccctggtgcagtgggtcccaagagggtctatccaaatcggacgagagggacctgcagctgtaacaagctgattccagcttttatgtgcgttttgcggggtaggtccccgctggctgcgacccactgcccgtccttccttcaagctgccaccagggggcacccgcggccaggtgatgcctgctcccaggactggagaagccgccaagcatccccaggctgacagtggtgtctaggcctgggctctcctgtcctgcctcccacccgccacttcctgcgtgcactttacacgccagccacgccctgtcctagtggtcccccacccgcctcactgtctctcgctccacagctggctctgctctgcccggctggaacctctgtccttgtttgcctcccgcaatgggtggggtgccctggggggctactatgactcaacctgttctgagcccttcactggggacctcaggtgtgtccagtggctgtgggtgtttctagaaggcatagaggtgtgccacctcccagttcactttgagcactgttctgagaacaacatgccccatggtcaggggtcccaagataaacagaccctgggtcctgccttccagggcctcagaggctcaggagagaagcaaggattccccccaggttcccacatccttgcagaccaaagcaaactcgaatcctggcaggctcccagttgctgccctcttattcctggtgaccccctctgacagttggtccggcccgcagagcgccgctgctgccgcgtggtggctgcccgatggcccctgccgtggggcttcctagcaccggcagaacgcagatgggcagcgtgctgtggagaggcagcagctctcctgctggactctctcagtctctggtccacattctaccctcgccaccgtgtgcttgaggaagcccctaagtcagctcggctggaaagtggttctaggaaactggccttcgtggccgctttggggatgagaccatgtgtgatgccttcagcaagatcccagttcgtatgcgcaggggtgccgcagtgatcctgcgtggactatcctattggcaggcctgcccttccccaggttacctacccggaggaatcccgcaggccctcccacaacaggcttcaacgccccctcctccatgaagttctccttgatctgtcctgcctggggggagagatttgtgccagccgagcgtgctcgggtgcgggagtcaaacacacctacacttgctctgaggagtcctgggcaaggctcccccaggggcccatgctgtatccctagggctgtttttttctctccggccgttttcttctctccatgcttccccatctccgctcctccctgcttcttcttacacactggcctcatcctctccctcttcagagatgacccccaaatcattcccctttccattatcctcagccagccaacccctcccagggactgtgtaaaactctcatggaaggatctgattggctctgtgtgggtcacttgcccactttttgcaccaatcagcatggacagggatatcacatgcccaagttggccaggccaaggggcgaggagagcactgtgattgaccgctcacagggatagggagtgcagtggcgccatctcggctcgctgcaacctccgcctcctggagtcaaacacttctcctgccgcagcctcccaagtagctgggattacaggcgccacgggtggcgagggcaggagcctttctttccccaaaggaaagaggagcggagcactggggctgaaagcagccggcgttgnggtcccacatgcagatgaggctaggagaggtgaagcagctcccctgcccttttccctgttaagggaaccttctggaatttaagaaacctgcctgaatgtgaggaatggctctcatggtggtgcggcctggtgctctaggatgagaggggcccctccctctcccccagagcacgtgtcagctgaattccacacccgaacagggggaggatcaggaactgtgtgggttgcaaatgactttaattatgtcgctctccttccactaaatggatcagaagaaccagcattgtgtgaaatcacccaggttcattctgtgaactgttccctgaagaacaaagggggctgcctccccactgtgctggcgcggggagggtgtggcctcccccagtcagcctgtggcgcctgggcagggcccttctgtgggactcgttcacccggcccccctcgcatgctgcttcccttgtcccaggctctgagtcacatataaagcgggtgcggctttggtcacatggacgattaggatcgaggagtacctggtaaataacaagaagcaaagtgcctcaagcccagcactgtgcggggtgttcagcctatgtcccctgattcacgctcaccaggggctgcgttcccttgttgatcccagttcttatagaaaactaagagagtgcgcatgtcagggtagggcagagctgggatccggacccaggcggactggccagagcccaagcccctgactgcagtgccggagctcacgtgtgagcttttgtcctctgtgcataacccctggagacccagggatccacctggttctgtcacagagcagtgtcaccctcaggaagcccagctcagcttccaaaaggaaacaggatttcctccctgaagagccttcagcaggacaacttctttatggtcgcttttcgtaacatcctcttcctccccagttaccttgcacctcacagctgctccagggccctgcagaggccaaaccccaaaaccctccctctgggcgcaggcccacagaactgtgctttctctcctgcctcttactatgtggatgaacttacccttcctcccaccggcaggaaccccttctccttaggagcagggagagcagagaaatggtgtggaattctctttagcgggacggtgagggcagcgagcccctgcacgtcagcactgctcgccccgaccctcatggcgatgcatgtttgtttattcgcccgagtcctcgggacaggcgggcccctggagggaagcagggcctggtggttttctgtcccagctgcttccatgtctacctcctgctgtggttgcacagtctgtttggaaggccagagctttctctcctggactaacagaaattacaggagggtcttttcttttcttttcttttcttttttcgagacggagttttgctcttattgcccaggctggagtgcagtggcgcgatcttggctcactacaaccttgcctcctggattcaagcaactctcctgcctcagcctcctgagtagctgggattacaggtgcccaccaccatgtccagctaatttttgtatttttagtagagacagggtttcatcatgttggccaggctggtctcaaactcctaacctcaggtgatctgcccgccttggcttcccaaagtgctgggattacaggtgtgagccaccacgcctggccaggagggtattttcatgatgattggggcagaggataaaaaggatcttagctcctggccccaaagtctgcatgttggtggcagctgacatgtgagggggtataagagaccccaagatacaggggaatagaggcaggtgtggtttccctttccagggagtgatggctctgtaggctctgggattgctttttcatttgtttgttgttttgggacagagttttactctgtcacccaggctagactgcagtggtaaatcacagctcactgcagtctctgcctccccaggctcaggtgatcctcctgcctccccaggctcaggtgatcctcctgcctcaacctcctgaatagctgggactacggtcatttttaatttttttttgtagaggtggagtctcgctatgttgtccaggctggtcttgaactcctaggctgaagcaatcctcccaccggggcctcccaaagtcctgggattatgggcgtgagcccccacacctgggcttatttctcgagaaggggcttgtgctcctcctcacctgatgcctctccttctcccaccagcgtcatcatgaccggggcctacaacaacttcttccgcatgttcgatcggaacaccaagcgggacgtgaccctggaggcctcgagggaaagcagcaagccccgggctgtgctcaagccacggcgcgtgtgcgtggggggcaagcgccggcgtgatgacatcagtgtggacagcttggacttcaccaagaagatcctgcacacggcctggcacccggctgagaacatcattgccatcgccgccaccaacaacctgtacatcttccaggacaaggtaaactctgacatgcactaggtatgtgcagttcccggcccctgccacccagcctcatgcaagtcatccccgacatgaccttcacgaccgcaatgcaaggaggggaagaaagtcacagcactgatgaggacagctgcagaggtggcagtgtgtggacacaggaagtttgggccccctccctgccccagctttcctaggccagaattgtgtttggcagtaattgtctgtttaaaaaaataaaaag

SEQ ID NO: 38 - protein, 447 aa, Homo sapiens [sequence omitted]

SEQ ID NO: 39 - DNA, 19 bp, Artificial
  primer 99-24169/139
ctggctgaggcctccttgt

SEQ ID NO: 40 - DNA, 19 bp, Artificial
  primer 24-257/320
gtccttctgatggcctgcc

SEQ ID NO: 41 - DNA, 19 bp, Artificial
  primer 99-24175/218
caagctggattcgcaatca

SEQ ID NO: 42 - DNA, 601 bp, Artificial
  amplicon 30-4
tgaagccgacgttgtcgccgggcagagcttcgctcagagcctcgtggtgcatctccactgacttcacctcagtggtgatgttcactggcgcaaaggtcaccaccatgcccggccgcaggatgccggtctccacccggcccacgggcaccgtgccaatgcctgcagaggggagggggtgtgaggggaaggtggggcccgaggggatgctggggcaggatattcggggacagagcctggaaaccaacaaagcctgggactggatccccccgacaggcctgggggttggggccacatgggcggrgtgcaggggaagggaggccagggacaagggcagacacagagattccaagggaagtgggggctctcccacccagctggggaaataagaggctgagcagcagagctcccaggaacccacggaaaagccacagggacagagaagcgggaggatgggcagagaggggctgtctgaacctggggtcccatccttgcccccggagagcactttccctcaaaggaggcactatgggacccctcctttgtctgaggactcctccctgtgagtgtgggcggggcgactgactgcttctgcctggggcct

SEQ ID NO: 43 - DNA, 601 bp, Artificial
  amplicon 30-2
gctaacaggagaaagcacctgcacactagctccccgacgctggaacagggccatggccctgcgccccacactccagctccactctccacaggaaaaggctcccagaatccagccactcagtgtgtgggggcaggggccctgctgacttagaaacaagtggcacattgatccgcattcaaacttgccagccaatcaaccacagccccgcgcacagactctcccaggtgggactgagggggtctcccctgtccttggcaggggcgtctcccccacgcacccccagtcccgtcctctccacagrctccagatgcccacatccccagaacactcaatgggacaactcagagcaggttacagagaaagaaaagccacacaagctcaccaagggcacgctatttcagaagtgccttctcctcctggaaatgtcgaccccaaagctctcactgggaaacctctggcctggccccgggaagcgacaggcgcaggtttggggctgaggccgtcccagcagctctgtggcctgccagacctcagagcactcccatcaggggccacaagagcagagagctcttcagccccatgttctcctggacgaattaaa

SEQ ID NO: 44 - DNA, 601 bp, Artificial
  amplicon 30-17
caccacccgccacctgcctccaggagcactgcagcctcgccagtcagcccactttgggctctgtctccagggatataggggctggatggacccgtctcctgaggccagcagaggctccacgccagggtcggtggcagggctggcacaggggaaccaggaggcgccgctggcttcaccatcttagctacggcagcccattcccctgagcctcctggcctgggcaacagtggctcgcatggccagcccaccgtgccctccagggtcagtagcgtctattctggcggccagcagggctggagartcttgggactgttgagaccctcccccaacctccctgagcctccgggcacagatgtgaaaagggtgcccactgcagtcagcactcaacccccacagcgtccagggagggagaggggccaccgggggctgacccctgcccattctgcagacaaagccaccaccctgccagggctcaagagggaagaaaatggggagggggccatttgagcaaatgagcccacccgtgagcaaggtggagggacagcacagtctggcaggatggggtctcggtcactggggggcctggggcccctggaactca

SEQ ID NO: 45 - DNA, 601 bp, Artificial
  amplicon 30-7
gccgagatcccccgagtcccgaggcgtcgcgtgcttggggacgtcaggagccgatggtacaggctcgctcaggaccccagtcctgagtccacacccctgcactgcctgaggccaacacaccgtgcccatgggggccaggggtgctcagagtcctggtgctgtgggtgcctctgtcccaacggcctctggtccccatcccaacaacaaaagcacaggtggtcggggagaaccggacgggggccaggggagcacatgggcacaggctcagcgggactcctggaatgttctctctttctccacygcacgagccatttcaaaggcaagaataggcccctcctgaccccgctcaggcaggcctcagggcaagtgggagtcactggaagactcaattcctctctctgcgtttccacccgaggcaggtccagtcaccagagagagaagcagccacctcctttctcacggcagctggcaaagcaccgggtggaggacagagccggtcggcccaactgtagcttcggggctgcccttggctggtctctgggcagagcccggtgctgagggcttgcagtgggaaaggcacagcttgaggaatgggcatcag

SEQ ID NO: 46 - DNA, 601 bp, Artificial
  amplicon 30-84
tgtgctaaaacatagtggcttaaaaataatgataaccatttatcgtctcagtttctgcagctcaggagtcggacggcacgcagccgatctccactcccaacgtgcagggcctctgccataagccttgaaggcactcattcactcacgcatcgggggctagtacaggctgtgacagaggcctgagctggaactgttgaccaggacacacacatggccatgtggcctctgggcttcctcacagcatggtgtctggattccaggagttggcatcctgagaaacaaccatgcagaagcagccctrtggatcctggcctggccttggagtcaggcagtgtcactcctgtgccttctaacctgggcccccgggcccaaggggaggaaatggagaccccacctcccagtggagggaaggcaaggtcccactgtgggggtagcacatgggatacacccatgtggctgccgctggagacgttagtttgccacacccgtttcttctacgtgaacatttgcctgcatctcaccttctaactcctgggtgctgtttgtccattttcactacaccaggggcccccacagtatatgcagaccaggtttcctggcc

SEQ ID NO: 47 - DNA, 601 bp, Artificial
  amplicon 30-15
gatgctgaataagccaggaagaagatccggctaaatgttggcacattctaaagtctacgtgaggccagtctgaccctgggaacctccatgaagacatgggcgtggagggtctgccttttgcagggcccaccaggggctcacaggaaaggtcgtggaaaattacaagaaatcttccctctggcactagcgggtgaggggaatggaagccaccgccagacagcaccatctcctcaccctcctgtgaagcacaagactcacttgcagagggaagagcgcagaaaccgtcaccccaggacgctgmggttgaacgagaggaagcgagaatggagaagccctggccctggggaacaggatggaaaacgcttggctcagctccgtggctgcgaaggaaccggcgcgctcgcggaggccacaccccgagacccgaggacacagtgcctgcctgagatggagccagaaacattctccaccctttcacgcaagactaacaagggctccatgaaaataaaactggaagagctgaaagagaagcattctccctgggtgtgaaaccaagaaaagacacaaagccaaggaaaagccattgagaaaacacctggca

(19) United States
(12) Patent Application Publication
(10) Pub. No.: US 2006/0275758 A1
(43) Pub. Date: Dec. 7, 2006

(54) Use of pp2a phosphatase modulators in the treatment of mental disorders

(75) Inventors: Daniel Cohen, Le Vesinet (FR); Iiya Chumakov, Vaux-le-Penil (FR); Marta Blumenfeld, Paris (FR); Sanober Shaikh, Paris (FR); Marta Palicio-Barron, Lille (FR); Hadi Abderrahim, Maisons-Alfort (FR); Pascale Grel, Viry-Chatillon (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Serono Genetics Institute S.A., Evry (FR)

(21) Appl. No.: 10/519,336

(22) PCT Filed: Jun. 20, 2003
(86) PCT No.: PCT/EP03/50247

§ 371 Date: Jul. 25, 2005

Related U.S. Application Data
(60) Provisional application No. 60/391,359, filed on Jun. 25, 2002.

(51) Int. Cl. C12Q 1/68; G01N 33/53; C12Q 1/42; C12P 19/34

(52) U.S. Cl. 435/6; 435/7.1; 435/21; 435/91.2

(57) ABSTRACT

The invention relates to the use of PP2A/Bγ and to the use of a PP2A phosphatase comprising the PP2A/Bγ subunit for screening for modulators thereof. The use of these modulators for treating mental disorders such as bipolar disorder, schizophrenia and depression, and drugs comprising these modulators are further disclosed. The invention also discloses biallelic markers located in the gene encoding PP2A/Bγ and their use for diagnosing mental disorders.

### FIELD OF THE INVENTION

The present invention is in the filed of mental disorders such as bipolar disorder, schizophrenia, depression and other mood disorders. More specifically, the invention relates to the use of a PP2A/Bγ subunit or of a PP2A phosphatase comprising the PP2A/Bγ subunit for screening for modulators, and to the use of said modulators for treating said mental disorders. The invention further relates to the use of biallelic markers located in the gene encoding the PP2A/Bγ subunit for diagnosing said mental disorders.

### BACKGROUND

1. The PP2A Phosphatase

The Protein Phosphatase 2A (PP2A) is one of the major intracellular serine/threonine protein phosphatases, and accounts for a large portion of the total phosphatase activity of some cells. In addition of its serine/threonine protein phosphatase activity, PP2A also exhibits low but detectable phosphotyrosine phosphatase activity. Although the precise functions of PP2A in vivo have not yet been determined, evidences suggest that PP2A plays a role in metabolism, DNA replication, cell proliferation, cell cycle and viral transformation. Moreover, PP2A deregulation has been suggested contribute to carcinogenesis and to the development of taupathies such as Alzheimer's disease (see, e.g., Janssens and Goris (2001) Biochem J. 353:417-439). Accordingly, PP2A plays a pivotal role in a wide variety of cellular processes.

PP2A is constituted by two or three subunits. PP2A phosphatases comprise of catalytic subunit (PP2A/C), a scaffolding subunit (PP2A/A) and eventually a regulatory subunit (PP2A/B). Two striking features of the B subunits are their diversity, stemming from the existence of entire subunit families, and the total lack of sequence similarity between the gene families, even though they recognize similar segments of the A subunit. As a consequence, at least 75 different dimeric and trimeric PP2A isoforms can be generated through combinational associations of different A, B and C subunits.

The existence of multiple families and isoforms of the PP2A/B subunits raises the possibility that different physiological functions are carried out by different isoforms. Indeed, biochemical characterization has demonstrated that subunit composition and specific complex formation play important roles in modulating the substrate specificity and catalytic activity of PP2A (see, e.g., Usui et al. (1988) J Biol Chem. 263:3752-3761). Regulatory subunits are also thought to confer tissue specificity, subcellular localization and developmental regulation to PP2A.

PP2A/Bγ is one of the alternative B subunits. PP2A/Bγ is encoded by the PPP2R2C gene that was mapped to human chromosome 4p16 between markers D4S2925 and D4S3007 (Hu et al., Genomics., 2000, 67:83-6). Strack et al. showed that in rats, the PP2A/Bγ subunit can only be detected in brain. Furthermore, PP2A/Bγ is enriched in the cytosqueletal fraction of the cell and is developmentally regulated (Strack et al. (1998) J Comp Neurol. 1998 392:515-527). This article further shows that compartmentalization of brain PP2A is regulated by different B subunits, the PP2A/Bγ subunit anchoring PP2A to cytoskeletal structures. Based on the localization of the enzyme and on the postnatal increase of PPP2R2C expression, which coincides with synapse formation, Strack et al. hypothesized that PP2A phosphatases comprising the PP2A/Bγ subunit may be involved in synaptic plasticity and in neurological disorders.

Although PP2A being a major phosphatase, the precise physiological role of most of the different PP2A isoforms is still unknown. Additional data on the specific B subunits and on the processes in which they are specifically involved would very likely lead to the discovery of new therapeutic agents.

2. KCNQ Potassium Channels

Malfunction in ion channels, due to mutations in genes encoding channel proteins or the presence of autoantibodies, are increasingly being implicated in causing disease conditions, termed channelopathies. For instance, dysfunction of potassium channels has been associated with the pathophysiology of a number of neurological disorders both affecting the central and peripheral nervous system (e.g., episodic ataxia, epilepsy, neuromyotonia, Parkinson's disease, congenital deafness, long QT syndrome). Potassium channels, which demonstrate a high degree of diversity and ubiquity, are fundamental in the control of membrane depolarisation and cell excitability. A common feature of potassium channelopathies is a reduction or loss of membrane potential repolarisation. Marketed potassium channels openers include for example flupirtine, an analgesic drug used for treating pain.

KCNQ polypeptides belong to the potassium channel family. KCNQ polypeptides associate to form homomeric or heteromeric potassium channels, each polypeptide corresponding to a subunit of the channel. Currently, five different members of the KCNQ family are known: KCNQ1, KCNQ2, KCNQ3, KCNQ4 and KCNQ5. Heteromeric KCNQ potassium channels can be comprised either of different members of the KCNQ family, or of KCNQ polypeptides associated with other members of the potassium channel family. Some of the KCNQ potassium channels, including KCNQ2 and KCNQ3, underlie the M-current, an important regulator of neuronal excitability. Both their amino-terminal and their carboxyl-terminal extremities are located on the intracellular side of the membrane. These extremities play an important role both in interactions with other proteins and in modulation of the channel's activity.

The activity of KCNQ channels has been shown to be modulated by the Protein kinase A (PKA) and by the c-Src tyrosine kinase (Src). Schroeder et al. showed that currents generated by heteromeric KCNQ2/KCNQ3 channels can be increased by intracellular cyclic AMP, and that this effect is mediated by the PKA kinase. PKA stimulated current intensity by 66% (Schroeder et al. (2000) Epilepsia (2000) 41:1068-1069). Gamper et al. showed that coexpression of Src with KCNQ2/KCNQ3 heteromeric channels resulted in a 4.5-fold reduction of current density and a 2-fold slowing of activation kinetics at 0 mV. However, Src had no effect on currents generated by KCNQ2 homomeric channels (Gamper et al. (2003) J Neurosci. 23:84-95). Accordingly, modulation of the phosphorylation state of KG NQ channels, which represents a balance between the activities of kinases and phosphatases, is believed to be important for control of neuronal excitability.

3. Mental Disorders

Mental disorders encompass a wide range of CNS disorders. Mental disorders include, e.g., mood disorders, psychotic disorders, anxiety disorders, childhood disorders, eating disorders and personality disorders, all these terms being defined according to the DSM-IV classification (Diagnosis and Statistical Manual of Mental Disorders, Fourth Edition, American Psychiatric Association, Washington D.C., 1994). Mood Disorders encompass bipolar I disorder (mania with or without major depression), bipolar II disorder (hypomania with major depression), cyclothymic disorder (numerous brief episodes of hypomania and minor depression), dysthymic disorder (prolonged minor depression without mania/hypomania) and major depressive disorder (major depression without mania). Psychotic disorders encompass schizophrenia, schizoaffective disorder, schizophreniform disorder, brief psychotic disorder, delusional disorder and shared psychotic disorder. Bipolar disorder, schizophrenia and depression are three particularly serious and widespread mental disorders.

3.1. Bipolar Disorder

Bipolar disorders are relatively common disorders, occurring in about 1.3% of the population, and have been reported to constitute about half of the mood disorders seen in psychiatric clinics with severe and potentially disabling effects. Bipolar disorders have been found to vary with gender depending of the type of disorder; for example, bipolar disorder I is found equally among men and women, while bipolar disorder II is reportedly more common in women. The age of onset of bipolar disorders is typically in the teenage years and diagnosis is typically made in the patient's early twenties. Bipolar disorders also occur among the elderly, generally as a result of a neurological disorder or other medical conditions. In addition to the severe effects on patients' social development, suicide completion rates among bipolar patients are reported to be about 15%.

Bipolar disorders are characterized by phases of excitement and often depression; the excitement phases, referred to as mania or hypomania, and depressive phases can alternate or occur in various admixtures, and can occur to different degrees of severity and over varying duration. Since bipolar disorders can exist in different forms and display different symptoms, the classification of bipolar disorder has been the subject of extensive studies resulting in the definition of bipolar disorder subtypes and widening of the overall concept to include patients previously thought to be suffering from different disorders. Bipolar disorders often share certain clinical signs, symptoms, treatments and neurobiological features with psychotic illnesses in general and therefore present a challenge to the psychiatrist to make an accurate diagnosis. Furthermore, because the course of bipolar disorders and various mood and psychotic disorders can differ greatly, it is critical to characterize the illness as early as possible in order to offer means to manage the illness over a long term.

The mania associated with the disease impairs performance and causes psychosis, and often results in hospitalization. This disease places a heavy burden on the patient's family and relatives, both in terms of the direct and indirect costs involved and the social stigma associated with the illness, sometimes over generations. Such stigma often leads to isolation and neglect. Furthermore, the earlier the onset, the more severe are the effects of interrupted education and social development.

The DSM-IV classification of bipolar disorder distinguishes among four types of disorders based on the degree and duration of mania or hypomania as well as two types of disorders which are evident typically with medical conditions or their treatments, or to substance abuse. Mania is recognized by elevated, expansive or irritable mood as well as by distractability, impulsive behavior, increased activity, grandiosity, elation, racing thoughts, and pressured speech. Of the four types of bipolar disorder characterized by the particular degree and duration of mania, DSM-IV includes:
bipolar disorder I, including patients displaying mania for at least one week;
bipolar disorder II, including patients displaying hypomania for at least 4 days, characterized by milder symptoms of excitement than mania, who have not previously displayed mania, and have previously suffered from episodes of major depression;
bipolar disorder not otherwise specified (NOS), including patients otherwise displaying features of bipolar disorder II but not meeting the 4 day duration for the excitement phase, or who display hypomania without an episode of major depression; and
cyclothymia, including patients who show numerous manic and depressive symptoms that do not meet the criteria for hypomania or major depression, but which are displayed for over two years without a symptom-free interval of more than two months.

The remaining two types of bipolar disorder as classified in DSM-VI are disorders evident or caused by various medical disorder and their treatments, and disorders involving or related to substance abuse. Medical disorders which can cause bipolar disorders typically include endocrine disorders and cerebrovascular injuries, and medical treatments causing bipolar disorder are known to include glucocorticoids and the abuse of stimulants. The disorder associated with the use or abuse of a substance is referred to as “substance induced mood disorder with manic or mixed features”.

Evidence from twin and adoption studies, and the lack of variation in incidence worldwide, indicate that bipolar disorder is primarily a genetic condition, although environmental risk factors are also involved at some level as necessary, sufficient, or interactive causes. Aggregation of bipolar disorder and schizophrenia in families suggests that these two distinct disorders share some common genetic susceptibility. Several linkage studies of bipolar disorder have been reported, and several susceptibility regions have been identified. The regions that are associated with bipolar disorder include 1q31-q32, 4p16, 7q31, 12q23-24, 13q32, 18p11.2, 21q22 and 22q11-q13 (Detera-Wadleigh et al. (1999) Proc Natl Acad Sci USA A96(10):5604-9). Some of these regions, like 4p16, 12q24, 18p11, 21q21 and 22q11 have been repeatedly implicated by independent investigators. Furthermore, some regions that are linked to bipolar disorder such as, e.g., 13q32 and 18p11.2, are also implicated in genome scans of schizophrenia, confirming that these two distinct disorders share some common genetic susceptibility. However, the genes underlying bipolar disorder and/or schizophrenia have not yet been identified.

3.2. Schizophrenia

There are an estimated 45 million people with schizophrenia in the world, with more than 33 million of them in the developing countries. In developed countries schizophrenia occurs in approximately 1% of the adult population at some point during their lives. If there is one grandparent with schizophrenia, the risk of getting the illness increases to about 3%; one parent with Schizophrenia, to about 10%. When both parents have schizophrenia, the risk rises to approximately 40%. Most schizophrenia patients are never able to work. Standardized mortality ratios (SMRs) for schizophrenic patients are estimated to be two to four times higher than the general population and their life expectancy overall is 20% shorter than for the general population. The most common cause of death among schizophrenic patients is suicide (in 10% of patients) which represents a 20 times higher risk than for the general population. Deaths from heart disease and from diseases of the respiratory and digestive system are also increased among schizophrenic patients.

Schizophrenia comprises a group of psychoses with either ‘positive’ or ‘negative’ symptoms. Positive symptoms consist of hallucinations, delusions and disorders o f thought; negative symptoms include emotional flattening, lack of volition and a decrease in motor activity.

A number of biochemical abnormalities have been identified and, in consequence, several neurotransmitter based hypotheses have been advanced over recent years; the most popular one has been “the dopamine hypothesis,” one variant of which states that there is over-activity of the mesolimbic dopamine pathways at the level of the D₂ receptor. However, researchers have been unable to consistently find an association between various receptors of the dopaminergic system and schizophrenia.

3.3. Depression

Depression is a serious medical illness that affects 340 million people worldwide. In contrast to the normal emotional experiences of sadness, loss, or passing mood states, clinical depression is persistent and can interfere significantly with an individual's ability to function. As a result, depression is the leading cause of disability throughout the world.

Symptoms of depression include depressed mood, diminished interest or pleasure in activities, change in appetite or weight, insomnia or hypersomnia, psycho-motor agitation or retardation, fatigue or loss of energy, feelings of worthlessness or excessive guilt, anxiety, inability to concentrate or act decisively, and recurrent thoughts of death or suicide. A diagnosis of unipolar major depression (or major depressive disorder) is made if a person has five or more of these symptoms and impairment in usual functioning nearly every day during the same two-week period. The onset of depression generally begins in late adolescence or early adult life; however, recent evidence suggests depression may be occurring earlier in life in people born in the past thirty years.

The World Health Organization predicts that by the year 2020 depression will be the greatest burden of ill-health to people in the developing world, and that by then depression will be the second largest cause of death and disability. Beyond the almost unbearable misery it causes, the big risk in major depression is suicide. Within five years of suffering a major depression, an estimated 25% of sufferers try to kill themselves. In addition, depression is a frequent and serious complication of heart attack, stroke, diabetes, and cancer. According to one recent study that covered a 13-year period, individuals with a history of major depression were four times as likely to suffer a heart attack compared to people without such a history. Depression may also be a feature in up to 50% of patients with mental disorders such as Parkinson's disease and Alzheimer's disease.

3.4. Treatment

There are currently no cures for mental disorders such as bipolar disorder, schizophrenia, depression and other mood disorders, so the objective of treatment is to reduce the severity of the symptoms, if possible to the point of remission. Due to the similarities in symptoms, schizophrenia, depression and bipolar disorder are often treated with some of the same medicaments.

3.4.1. Treatment of Bipolar Disorder

Depressive episodes may be treated like depression. However, most antidepressants can cause swings from depression to hypomania or mania and sometimes cause rapid cycling between them. Therefore, these drugs are used for only short periods, and their effect on mood is closely monitored. At the first sign of a swing to hypomania or mania, the antidepressant is stopped. Most people with manic-depressive disorder are given drugs with a mood-stabilizing effect such as lithium, carbamazepine and divalproex.

Lithium has no effect on normal mood but reduces the tendency toward mood swings in about 70% of the people with manic-depressive illness. A doctor monitors the level of lithium in the blood with blood tests. Possible adverse effects of lithium include tremor, muscle twitching, nausea, vomiting, diarrhea, thirst, excessive urination, and weight gain. Lithium can make acne or psoriasis worse, can cause the blood levels of thyroid hormone to fall, and rarely can cause excessive urination. A very high level of lithium in the blood can cause a persistent headache, mental confusion, drowsiness, seizures, and abnormal heart rhythms. Adverse effects are more likely to occur in the elderly. Women who are trying to become pregnant must stop taking lithium, because lithium may cause heart defects in a developing fetus.

Newer drug treatments have evolved over the past several years. These include the carbamazepine and divalproex. However, carbamazepine can seriously reduce the number of red and white blood cells, and divalproex can cause liver damage (primarily in children). With careful monitoring by a doctor, these problems are rare, and carbamazepine and divalproex are useful alternatives to lithium, especially for people with the mixed or rapid cycling form of manic-depressive illness who haven't responded to other treatments.

3.4.2. Treatment of Schizophrenia

For schizophrenia, antipsychotic medications are the most common and most valuable treatments. There are four main classes of antipsychotic drugs which a re commonly prescribed for schizophrenia. The first, neuroleptics, exemplified by chlorpromazine (Thorazine), has revolutionized the treatment of schizophrenic patients by reducing positive (psychotic) symptoms and preventing their recurrence. Patients receiving chlorpromazine have been able to leave mental hospitals and live in community programs or their own homes. But these drugs are far from ideal. Some 20% to 30% of patients do not respond to them at all, and others eventually relapse. These drugs were named neuroleptics because they produce serious neurological side effects, including rigidity and tremors in the arms and legs, muscle spasms, abnormal body movements, and akathisia (restless pacing and fidgeting). These side effects are so troublesome that many patients simply refuse to take the drugs. Besides, neuroleptics do not improve the so-called negative symptoms of schizophrenia and the side effects may even exacerbate these symptoms. Thus, despite the clear beneficial effects of neuroleptics, even some patients who have a good short-term response will ultimately deteriorate in overall functioning.

The well known deficiencies in the standard neuroleptics have stimulated a search for new treatments and have led to a new class of drugs termed atypical neuroleptics. The first atypical neuroleptic, Clozapine, is effective for about one third of patients who do not respond to standard neuroleptics. It seems to reduce negative as well as positive symptoms, or at least exacerbates negative symptoms less than standard neuroleptics do. Moreover, it has beneficial effects on overall functioning and may reduce the chance of suicide in schizophrenic patients. It does not produce the troubling neurological symptoms of the standard neuroleptics, or raise blood levels of the hormone prolactin, excess of which may cause menstrual irregularities and infertility in women, impotence or breast enlargement in men. Many patients who cannot tolerate standard neuroleptics have been able to take clozapine. However, clozapine has serious limitations. It was originally withdrawn from the market because it can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus only patients who do not respond to other drugs usually take Clozapine.

Researchers have developed a third class of antipsychotic drugs that have the virtues of clozapine without its defects. One of these drugs is risperidone (Risperdal). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as safer. Another new drug is Olanzapine (Zyprexa) which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases, liver function tests become transiently abnormal.

3.4.3. Treatment of Depression

Several types of antidepressants are available. These antidepressants belong to four main categories: tricyclic antidepressants, selective serotonin reuptake inhibitors, monoamine oxidase inhibitors and psychostimulants. Tricyclic antidepressants include, e.g., Amitriptyline, Amoxapine, Bupropion, Clomipramine, Desipramine, Doxepin, Imipramine, Maprotiline, Nefazodone, Nortriptyline, Protriptyline, Trazodone, Trimipramine and Venlafaxine. Selective serotonin reuptake inhibitors include, e.g., Fluoxetine, Fluvoxamine, Paroxetine and Sertraline. Monoamine oxidase inhibitors include, e.g., Isocarboxazid, Pargyline, Phenelzine and Tranylcypromine. Psychostimulants include, e.g., Dextroamphetamine and Methylphenidate.

All these antidepressants must be taken regularly for at least several weeks before they begin to work. The chances that any given antidepressant will work for a particular person are about 65%. However, most of these drugs have side effects varying with each type of drug. For example, the tricyclic antidepressants often cause sedation and lead to weight gain. They can also be associated with side effects such as an increased heart rate, a decrease in blood pressure when the person stands or blurred vision.

Thus, for mental disorders such as bipolar disorder, schizophrenia, depress ion and other mood disorders, known molecules used for the treatment have side effects and act only against the symptoms of the disease. Consequently, there is a strong need for new molecules without associated side effects that are specifically directed against targets which are involved in the causal mechanisms of such mental disorders. Therefore, there is a need to identify proteins involved in bipolar disorder and schizophrenia. Providing new targets involved in bipolar disorder and schizophrenia will allow new screenings for drugs, resulting in new drugs that are efficient in treatment of these serious mental disorders.

Furthermore, there is also a need for diagnostic tools. There is increasing evidence that leaving schizophrenia untreated for long periods early in course of the illness may negatively affect the outcome. However, the use of drugs is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia or bipolar disorder is difficult to distinguish from, e.g., drug-related disorders and stress-related disorders. Accordingly, there is a need for new methods for detecting a susceptibility to mental disorders such as bipolar disorder, schizophrenia, and depression.

### SUMMARY OF THE INVENTION

The present invention is based on the finding that PP2A/Bγ deregulation is associated with development of bipolar disorder.

Therefore, a first aspect of the present invention is directed to the use of a PP2A/Bγ subunit as a target for screening candidate modulators.

A second aspect of the present invention is directed to the use of a PP2A phosphatase comprising a PP2A/Bγ subunit as a target for screening candidate modulators.

In a third aspect, the invention relates to the use of modulator of a PP2A phosphatase comprising a PP2A/Bγ subunit for preparing a medicament for the treatment of a mental disorder, and to the use of a gene therapy vector comprising a polynucleotide encoding a PP2A/Bγ subunit for preparing a medicament for the treatment of a mental disorder.

The use of a PP2A/Bγ subunit as a target for screening for natural binding partners is a fourth aspect of the present invention.

A fifth aspect of the invention pertains to a method of assessing the efficiency of a modulator of a PP2A phosphatase comprising a PP2A/Bγ subunit for the treatment of a mental disorder, said method comprising administering said modulator to an animal model for said mental disorder; wherein a determination that said modulator ameliorates a representative characteristic of said mental disorder in said animal model indicates that said agonist is a drug for the treatment of said mental disorder.

In the frame of the present invention, biallelic markers located in the gene encoding PP2A/Bγ have been identified.

Therefore, a sixth aspect of the invention relates to the use of at least one PP2A/Bγ-related biallelic marker for diagnosing whether an individual suffers from or is at risk of suffering from a mental disorder.

In a seventh aspect, the invention encompasses the use of at least one PP2A/Bγ-related biallelic marker for determining whether there is a significant association between said marker and a mental disorder.

In a eighth aspect, the invention relates to a method of genotyping comprising the step of determining the identity of a nucleotide at a PP2A/Bγ-related biallelic marker or the complement thereof in a biological sample.

A ninth aspect of the present invention pertains to a method of diagnosing a mental disorder in an individual comprising the step of genotyping at least one PP2A/Bγ-related biallelic marker according to the method of any of claims 29 to 33.

In a tenth aspect, the invention pertains to the use of a polynucleotide comprising a contiguous span of at least 12 nucleotides of SEQ ID NO: 37 or a polynucleotide complementary thereto in a microsequencing assay for determining the identity of the nucleotide at a PP2A/Bγ-related biallelic marker, wherein the 3′ end of said polynucleotide is located 1 nucleotide upstream of said PP2A/Bγ-related biallelic marker in said sequence.

### BRIEF DESCRIPTION OF THE FIGURES

FIGS. 1A and 1B show an alignment between the full-length KCNQ2 polypeptide (KCNQ2-fl, SEQ ID NO: 7), KCNQ2-15bx (SEQ ID NO: 2), KCNQ2-15by (SEQ ID NO: 4) and KCNQ2-15bz (SEQ ID NO: 6). The box shows highlights the amino acids that are unique to KCNQ2-15bx, KCNQ2-15by and KCNQ2-15bz compared to KCNQ2-fl.

FIG. 2 shows a sheme of the structure of the KCNQ2-15bx, KCNQ2-15by and KCNQ2-15bz cDNAs.

FIG. 3 shows the results of a mating test between PP2A/Bγ and different KCNQ2 polypeptides, as described in detail in Example 3.

FIG. 4 shows the results of a mating test between different KCNQ2 polypeptides, as described in detail in Example 3.

FIG. 5 compares the intensity of the currents generated by homotetrameric potassium channels comprised of KCNQ2-15bx, KCNQ2-15by, KCNQ2-15bz or KCNQ2-fl subunits respectively.

FIG. 6A shows the voltage clamp traces of the current generated by a homotetrameric potassium channels comprised of KCNQ2-15bx subunits.

FIG. 6B shows the voltage clamp traces of the current generated by a homotetrameric potassium channels comprised of KCNQ2-15by subunits.

### BRIEF DESCRIPTION OF THE SEQUENCES OF THE SEQUENCE LISTING

SEQ ID NO: 1 corresponds to a polynucleotide consisting of the CDS of KCNQ2-15bx

SEQ ID NO: 2 corresponds to the KCNQ2-15bx polypeptide.

SEQ ID NO: 3 corresponds to a polynucleotide consisting of the CDS of KCNQ2-15by

SEQ ID NO: 4 corresponds to the KCNQ2-15by polypeptide.

SEQ ID NO: 5 corresponds to a polynucleotide consisting of the CDS of KCNQ2-15bz

SEQ ID NO: 6 corresponds to the KCNQ2-15bz polypeptide.

SEQ ID NO: 7 corresponds to the KCNQ2-fl polypeptide.

SEQ ID Nos. 8 to 36 correspond to primers and probes used in Examples 1 to 4.

SEQ ID NO: 37 corresponds to the PPP2R2C gene which encodes the PP2A/Bγ subunit, on which PP2A/Bγ-related biallelic markers are indicated.

SEQ ID NO: 38 corresponds to the PP2A/Bγ subunit.

SEQ ID Nos. 39 to 41 correspond to primers used for microsequencing some of the PP2A/Bγ-related biallelic markers.

SEQ ID Nos. 42 to 47 correspond to regions of the KCNQ2 gene, on which KCNQ2-related biallelic markers are indicated.

### BRIEF DESCRIPTION OF THE TABLES

Table 1 presents the structure of KCNQ2-fl, KCNQ2-15bx KCNQ2-15by and KCNQ2-15bz.

Tables 2A and 2B present the location of the primers used for amplification of genomic DNA by PCR in the PPP2R2C and in the KCNQ2 gene respectively

Table 3A and 3B present biallelic markers located in PP2R2C and in the KCNQ2 gene respectively.

Tables 4A and 4B present the the primers used for microsequencing biallelic markers located in PP2R2C and in the KCNQ2 gene respectively.

Tables 5A and 5B present the p-values for biallelic markers located in PPP2R2C and in the KCNQ2 gene respectively.

Tables 6A and 6B present the genotypic odds ratios for biallelic markers located in PPP2R2C and in the KCNQ2 gene respectively.

Tables 7A and 7B present the risk haplotypes for two sets of biallelic markers located in PPP2R2C

### DETAILED DESCRIPTION OF THE INVENTION

The present invention stems from association studies between the gene encoding PP2A/Bγ (PPP2R2C) and bipolar disorder. As shown in example 15, PPP2R2C is strongly associated with bipolar disorder in two different populations. Novel validated biallelic markers located in PPP2R2C and associated with bipolar disorder are provided. In the frame of the present invention, it was further demonstrated that PP2A/Bγ interacts with novel splice variants of the KCNQ2 potassium channel, and that the KCNQ2 gene is also associated with bipolar disorder. Moreover, it was shown that (i) PP2A dephosphorylates the novel KCNQ2 splice variants; and (ii) GSK3β and PKA phosphorylate the novel KCNQ2 splice variants. In addition, phosphorylation of the novel KCNQ2 splice variants is inhibited in the presence of lithium, a known mood-stabilizing agent.

Accordingly, the present invention provides means to identify compounds useful in the treatment of mental disorders such as bipolar disorder, schizophrenia, depression and other mood disorders. The invention further relates to the use of PP2A/Bγ or to the use of a PP2A phosphatase comprising PP2A/Bγ as a target for screening for modulators thereof, and to the use of said modulators for treating mental disorders. The invention also relates to the use of biallelic markers located in PPP2R2C gene for diagnosing mental disorders.

1. Definitions

The term “treat” or “treating” as used herein is meant to ameliorate, alleviate symptoms, eliminate the causation of the symptoms either on a temporary or permanent basis, or to prevent or slow the appearance of symptoms of the named disorder or condition. The term “treatment” as used herein also encompasses the term “prevention of the disorder”, which is, e.g., manifested by delaying the onset of the symptoms of the disorder to a medically significant extent. Treatment of the disorder is, e.g., manifested by a decrease in the symptoms associated with the disorder or an amelioration of the reoccurrence of the symptoms of the disorder.

The term “mental disorder” refers to diseases characterized as mood disorders, psychotic disorders, anxiety disorders, childhood disorders, eating disorders, personality disorders, adjustment disorder, autistic disorder, delirium, dementia, multi-infarct dementia and Tourette's disorder in the DSM-IV classification (Diagnosis and Statistical Manual of Mental Disorders, Fourth Edition, American Psychiatric Association, Washington D.C., 1994).

The term “schizophrenia” refers to a condition characterized as schizophrenia in the DSM-IV classification (Diagnosis and Statistical Manual of Mental Disorders, Fourth Edition, American Psychiatric Association, Washington D.C., 1994).

The term “bipolar disorder” as used herein refers to a condition characterized as a Bipolar Disorder in the DSM-IV. Bipolar disorder may be bipolar I and bipolar disorder II as described in the DSM-IV. The term further includes cyclothymic disorder. Cyclothymic disorder refers to an alternation of depressive symptoms and hypomanic symptoms. The skilled artisan will recognize that there are alternative nomenclatures, posologies, and classification systems for pathologic psychological conditions and that these systems evolve with medical scientific progress.

The terms “comprising”, “consisting of”, or “consisting essentially of” have distinct meanings. However, each term may be substituted for another herein to change the scope of the invention.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe compounds comprising RNA, DNA, or RNA/DNA hybrid sequences of any length in single-stranded or duplex form. The term “nucleotide” is also used herein as a noun to refer to individual nucleotides or varieties of nucleotides, meaning a compound, or individual unit in a larger nucleic acid compound, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064, the disclosure of which is incorporated herein by reference. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “complementary” or “complement thereof” are used herein to refer to the sequences of polynucleotides which are capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “polypeptide” refers to a polymer of amino acids without regard to the length of the polymer; thus, peptides, oligopeptides, and proteins are included within the definition of polypeptide. This term also does not specify or exclude prost-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

As used herein, the term “exon” refers as well to the portion of a DNA that codes for portion of spliced mRNA as to the amino acids encoded by said part of a DNA.

As used herein, “splice variants” refer to different mRNAs produced by alternative splicing events and translated from the same gene. The term splice variant refers as well to the mRNA as to the corresponding polypeptide.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to symptoms of, or susceptibility to bipolar disorder; or to refer to an individual's response to an agent acting on bipolar disorder; or to refer to symptoms of, or susceptibility to side effects to an agent acting on bipolar disorder.

As used herein, the term “allele” refers to one of the variant forms of a biallelic marker, differing from other forms in its nucleotide sequence. Typically the first identified allele is designated as the original allele whereas other alleles are de signated as alternative alleles. Diploid organisms may be homozygous or heterozygous for an allelic form.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change. Typically a single nucleotide polymorphism is the replacement of one nucleotide by another nucleotide at the polymorphic site. A “single nucleotide polymorphism” (SNP) refers to a sequence polymorphism differing in a single base pair.

2. Uses of PP2A/Bγ and PP2A Phosphatases Comprising a PP2A/Bγ Subunit.

The present invention is directed to uses of the PP2A/Bγ subunit and of PP2A phosphatases comprising a PP2A/Bγ subunit for treating or diagnosing mental disorders.

A first aspect of the present invention is directed to the u se of a PP2A/Bγ subunit as a target for screening candidate modulators. As used herein, the term “PP2A/Bγ subunit” is used interchangeably with “PP2A/Bγ” and refers to a polypeptide encoded by the PPP2R2C gene. Thus the term PP2A/Bγ encompasses all variants that are encoded by PPP2R2C corresponding to, e.g., alternative splice variants, or polypeptides translated from alternative start methionines. A preferred PP2A/Bγ variant is the polypeptide of SEQ ID NO: 38.

As used herein, a “PP2A/Bγ modulator” refers to a compound that increases or decreases the activity of a PP2A/Bγ polypeptide and/or to a compound that increases or decreases the transcription level of the PP2A/Bγ mRNA encoding said polypeptide. The term “modulator” encompasses both agonists and antagonists.

As used herein, a “PP2A/Bγ antagonist” refers to a compound that decreases the activity of a PP2A/Bγ polypeptide and/or to a compound that decreases the expression level of the PP2A/Bγ mRNA encoding said polypeptide. The terms “antagonist” and “inhibitor” are considered to be synonymous and can be used interchangeably throughout the disclosure.

As used herein, a “PP2A/Bγ agonist” refers to a compound that increases the activity of a PP2A/Bγ polypeptide and/or to a compound that increases the expression level of the PP2A/Bγ mRNA encoding said polypeptide. The terms “agonist” and “activator” are considered to be synonymous and can be used interchangeably throughout the disclosure.

Methods that can be used for testing modulators for their ability to increase or decrease the activity of a PP2A/Bγ polypeptide or to increase or decrease the expression of a PP2A/Bγ mRNA are well known in the art and further detailed below.

PP2A/Bγ is one of the alternative regulatory B subunits of the PP2A phosphatase. As shown in examples 4 and 6, a PP2A/Bγ subunit is capable of binding to KCNQ2 polypeptides in vitro. As used herein, the term “KCNQ2 polypeptide” refers to any polypeptide encoded by the KCNQ2 gene. Thus the term “KCNQ2 polypeptide” encompasses all alternative splice variants encoded by the KCNQ2 gene, such as, e.g., a polypeptide of SEQ ID NO: 2, a polypeptide of SEQ ID NO: 4, a polypeptide of SEQ ID NO: 6, a polypeptide of SEQ ID NO: 7 and other previously described isoforms (see, e.g., SwissProt Accession. No. O43526). Polypeptides comprising exon 15b as depicted in Example 2, such as polypeptides of SEQ ID Nos. 2, 4 and 6, are further defined as “KCNQ2-15b polypeptides”.

Thus the term “PP2A/Bγ activity” as used herein may refer to the capacity of PP2A/Bγ to bind to KCNQ2 polypeptides. Preferably, PP2A/Bγ refers to the capacity of PP2A/Bγ to bind KCNQ2-15b polypeptides. Alternatively, the term “PP2A/Bγ-activity” may refer to the capacity of PP2A/Bγ to bind to other subunits of the PP2A phosphatase such as a catalytic or a scaffolding subunit. The capacity of PP2A/Bγ to bind KCNQ2 polypeptides or to bind other subunits of the PP2A phosphatase may be assessed by several assays well known by those of skill in the art including, e.g., the yeast mating test described in example 4 and the solid phase overlay assay described in example 6.

A second aspect of the present invention is directed to the use of a PP2A phosphatase comprising a PP2A/Bγ subunit as a target for screening candidate modulators. As used herein, the term “modulator of a PP2A phosphatase comprising a PP2A/Bγ subunit” as used herein refers to a to a compound that increases or decreases any of the activities of a PP2A phosphatase comprising a PP2A/Bγ subunit. Modulators encompass both agonists, i.e., compounds that increase the activity of a PP2A phosphatase comprising a PP2A/Bγ subunit, and antagonists, i.e., compounds that decrease the activity of a PP2A phosphatase comprising a PP2A/Bγ subunit. A PP2A/Bγ modulator is believed to modulate the activity of a PP2A phosphatase comprising a PP2A/Bγ subunit. Accordingly, as used herein, the term “modulator of a PP2A phosphatase comprising a PP2A/Bγ subunit” encompasses the term “PP2A/Bγ modulator”.

The term “activity of a PP2A phosphatase comprising a PP2A/Bγ subunit” as used herein refers to the enzymatic activity of such a PP2A isoform. The activity of PP2A refers both to the serine/threonine protein phosphatase activity and to the phosphotyrosine phosphatase activity. The activity of a PP2A phosphatase comprising a PP2A/Bγ subunit may be assessed by several assays well known by those of skill in the art including, e.g., the dephosphorylation assay described in Example 7.

In all aspects and embodiments of the present invention, the modulator preferably specifically modulates a PP2A phosphatase comprising the PP2A/Bγ subunit. In other words, the modulator (i) increases or decreases the activity of a PP2A phosphatase comprising the PP2A/Bγ subunit; and (ii) has no or significantly less effect on the activity of a PP2A phosphatase that does not comprise the PP2A/Bγ subunit.

The assays for measuring the activity of PP2A/Bγ or of a PP2A phosphatase comprising a PP2A/Bγ subunit when screening for a modulator may be performed either in vitro or in vivo, as further detailed below.

Candidate compounds according to the present invention include naturally occurring and synthetic compounds. Such compounds include, e.g., natural ligands, small molecules, antisense mRNAs, antibodies, aptamers and short interfering RNAs. As used herein, the term “natural ligand” refers to any signaling molecule that binds to a phosphatase comprising PP2A/Bγ in vivo and includes molecules such as, e.g., lipids, nucleotides, polynucleotides, amino acids, peptides, polypeptides, proteins, carbohydrates and inorganic molecules. As used herein, the term “small molecule” refers to an organic compound. As used herein, the term “antibody” refers to a protein produced by cells of the immune system or to a fragment thereof that binds to an antigen. As used herein, the term “antisense mRNA” refers an RNA molecule complementary to the strand normally processed into m RNA and translated, or complementary to a region thereof. As used herein, the term “aptamer” refers to an artificial nucleic acid ligand (see, e.g., Ellington and Szostak (1990) Nature 346:818-822). As used herein, the term “short interfering RNA” refers to a double-stranded RNA inducing sequence-specific posttranscriptional gene silencing (see, e.g., Elbashir et al. (2001) Genes Dev. 15:188-200).

Such candidate compounds can be obtained using any of the numerous approaches in combinatorial library methods known in the art, including, e.g., biological libraries, spatially addressable parallel solid phase or solution phase libraries, and synthetic library methods using affinity chromatography selection. The biological library approach is generally used with peptide libraries, while the other four approaches are applicable to peptide, non-peptide oligomers, or small molecule libraries of compounds.

One example of a method that may be used for screening candidate compounds for a modulator is a method comprising the steps of.
a) contacting PP2A/Bγ or a PP2A phosphatase comprising a PP2A/Bγ subunit with the candidate compound; and
b) testing the activity of PP2A/Bγ or of the PP2A phosphatase comprising a PP2A/Bγ subunit in the presence of said candidate compound,
   wherein a difference in the activity of PP2A/Bγ or of the PP2A phosphatase comprising a PP2A/Bγ subunit in the presence of said compound in comparison to the activity in the absence of said compound indicates that the compound is a modulator of PP2A/Bγ or of the PP2A phosphatase comprising a PP2A/Bγ subunit.

Alternatively, the assay may be a cell-based assay comprising the steps of:
a) contacting a cell expressing PP2A/Bγ or a PP2A phosphatase comprising a PP2A/Bγ subunit with the candidate compound; and
b) testing the activity of PP2A/Bγ or of the PP2A phosphatase comprising a PP2A/Bγ subunit polypeptide in the presence of said candidate compound,
   wherein a difference in the activity of PP2A/Bγ or of the PP2A phosphatase comprising a PP2A/Bγ subunit in the presence of said compound in comparison to the activity in the absence of said compound indicates that the compound is a modulator of said KCNQ2 polypeptide.

The modulator may modulate any activity of said PP2A/Bγ or of said PP2A phosphatase comprising a PP2A/Bγ subunit. The modulator may for example modulate PP2A/Bγ mRNA expression within a cell, or modulate the binding of PP2A/Bγ to KCNQ2 polypeptides or to other subunits of PP2A. Further activities that may be measured include the serine/threonine protein phosphatase activity and the phosphotyrosine phosphatase activity of a PP2A phosphatase comprising a PP2A/Bγ subunit.

In a preferred embodiment, the activity of a PP2A phosphatase comprising a PP2A/Bγ subunit is assessed by measuring its serine/threonine protein phosphatase activity. This activity may be measured as described in Example 7. Several other methods for measuring the serine/threonine protein phosphatase activity of PP2A are well known in the art. Such methods include, e.g., the phosophatase assays described by Price et al. (Biochemistry. (2000) 39:11312-11318) and by Kamibayashi et al. (J Biol Chem. (1994) 269:20139-20148). Any known PP2A substrate may be used in such assays. In one embodiment, said substrate is KCNQ2 polypeptides.

In another preferred embodiment, the activity of a PP2A phosphatase comprising a PP2A/Bγ subunit is assessed by measuring its phosphotyrosine phosphatase activity. This activity may for example be measured as described by Agostinis et al. (Eur J Biochem. (1996) 236:548-557).

In a further preferred embodiment, the activity of PP2A/Bγ is assessed by measuring the binding of PP2A/Bγ to KCNQ2 polypeptides. The binding of PP2A/Bγ to KCNQ2 polypeptides can for example be measured by the yeast mating test as described in example 3 or by the solid phase overlay assay as described in example 6.

In a further preferred embodiment, the activity of PP2A/Bγ is assessed by measuring the binding of PP2A/Bγ to other the catalytic subunit of PP2A or to the scaffolding subunit of PP2A. This assay may also be performed using, e.g., the yeast mating test or the solid phase overlay assay described in examples 3 and 6.

In a further preferred embodiment, the activity of PP2A/Bγ is assessed by measuring the levels of PP2A/Bγ mRNA within a cell. In this embodiment, the activity can for example be measured using Northern blots, RT-PCR, quantitative RT-PCR with primers and probes specific for PP2A/Bγ mRNAs. The term “PP2A/Bγ mRNA” as used herein encompasses all alternative variants and splice variants translated from the PPP2R2C gene wich encodes PP2A/Bγ. The primers and probes may detect one specific PP2A/Bγ splice variant or detect all alternative splice variants translated from PPP2R2C. Alternatively, the expression of the PP2A/Bγ mRNA is measured at the polypeptide level, by using labeled antibodies that specifically bind to PP2A/Bγ in immunoassays such as ELISA assays, RIA assays, Western blots or immunohistochemical assays.

As shown in Example 15, deregulation of PP2A phosphatases comprising the PP2A/Bγ regulatory subunit contributes to the onset and to the development of bipolar disease. Accordingly, modulators of PP2A/Bγ or of a PP2A phosphatase comprising PP2A/Bγ which may be found, e.g., by any of the above screenings, are candidate drugs for the treatment of a mental disorder.

As used throughout the disclosures of the present specification, the term “mental disorder” is used as defined in the DSM-IV classification (Diagnosis and Statistical Manual of Mental Disorders, Fourth Edition, American Psychiatric Association, Washington D.C., 1994). In all aspects and embodiments of the present invention, the term “mental disorder” preferably refers to a mental disorder selected from the group consisting of bipolar disorder, schizophrenia and depression. In all aspects and embodiments, the term “mental disorder” most preferably refers to bipolar disorder.

A further aspect of the present invention is the use of modulator of a PP2A phosphatase comprising a PP2A/Bγ subunit for preparing a medicament for the treatment of a mental disorder. Such a medicament comprises said modulator of a KCNQ2 polypeptide in combination with any physiologically acceptable carrier. Physiologically acceptable carriers can be prepared by any method known by those skilled in the art. Physiologically acceptable carriers include but are not limited to those described in Remington's Pharmaceutical Sciences (Mack Publishing Company, Easton, USA 1985). Pharmaceutical compositions comprising a modulator of a PP2A phosphatase comprising a PP2A/Bγ subunit and a physiologically acceptable carrier can be for, e.g., intravenous, topical, rectal, local, inhalant, subcutaneous, intradermal, intramuscular, oral, intrathecal and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

If an increase of the activity of a PP2A phosphatase comprising a PP2A/Bγ subunit is sought in a patient, a particularly efficacious medicament is a gene therapy vector comprising a polynucleotide encoding PP2A/Bγ. Upon administration to a patient, such a vector will cause the active agent to be expressed in vivo, preferably specifically in the appropriate cells or tissues. Thus another aspect of the present invention is the use of a gene therapy vector comprising a polynucleotide encoding a PP2A/Bγ subunit for preparing a medicament for the treatment of a mental disorder.

Expression vectors that may be used for gene therapy are well known in the art, and they comprise further elements serving for expression of the gene of interest. They may comprise regulatory sequence, such as promoter and enhancer sequences, selection marker sequences, origins of multiplication, and the like. Advantageously, the expression of PP2A/Bγ will then be in situ, e.g., restricted to brain or to some regions of brain.

In a preferred embodiment, the expression vector is a lentiviral derived vector. Lentiviral vectors have been shown to be very efficient in the transfer of genes, in particular within the CNS. Other well established viral vectors, such as adenoviral derived vectors, may also be used according to the invention.

In a preferred embodiment of the invention, the expression vector may be administered by intramuscular injection.

The use of a vector for inducing and/or enhancing the endogenous production of PP2A/Bγ in a cell normally silent for expression of PP2A/Bγ, or which expresses amounts of PP2A/Bγ which are not sufficient, are also contemplated according to the invention. The vector may comprise regulatory sequences functional in the cells desired to express PP2A/Bγ. Such regulatory sequences may be promoters or enhancers, for example. The regulatory sequence may then be introduced into the appropriate locus of the genome by homologous recombination, thus PP2A/Bγ linking the regulatory sequence with the gene, the expression of which is required to be induced or enhanced. The technology is usually referred to as “endogenous gene activation” (EGA), and it is described e.g. in WO 91/09955.

Such medicaments comprising either a modulator of a PP2A phosphatase comprising a PP2A/Bγ subunit or a gene therapy vector comprising a polynucleotide encoding a PP2A/Bγ subunit may be administered in combination with any known drug for the treatment of a mental disorder. The modulator may for example be administered in combination with a mood-stabilizing drug used for treating bipolar disorder such as, e.g., lithium, carbamazepine or divalproex. The modulator may also be administered in combination with an antidepressant such as, e.g., a tricyclic antidepressant, a selective serotonin reuptake inhibitor, a monoamine oxidase inhibitor or a psychostimulant. When treating schizophrenia and other psychotic disorders, the modulator may for example be administered in combination with an antipsychotic drugs such as, e.g., chlorpromazine, clozapine, risperidone or olanzapine.

Another aspect is the use of a PP2A/Bγ subunit as a target for screening for natural binding partners. Methods for screening for natural binding partners include, e.g., the yeast two-hybrid screening that is described in Example 1. Using a PP2A/Bγ as a target has a utility for the identification of proteins involved in bipolar disorder and for providing new intervention points in the treatment of bipolar disorder and other mental disorders.

A method of assessing the efficiency of a modulator of a PP2A phosphatase comprising a PP2A/Bγ subunit for the treatment of a mental disorder, said method comprising administering said modulator to an animal model for said mental disorder; wherein a determination that said modulator ameliorates a representative characteristic of said mental disorder in said animal model indicates that said agonist is a drug for the treatment of said mental disorder is also contemplated according to the invention.

Animal models for mental disorders and assays for determining whether a compound ameliorates a representative characteristic of said mental disorder in said animal model are currently used and described in scientific and patent literature. For example, animal models that may be used in the above method include but are not limited to the conditioned avoidance behaviour model in rats, which is a standard behavioural test predictive of antipsychotic activity, the behavioral activity assessment of mice and rats in the Omnitech Digiscan animal activity monitors, the purpose of which is to evaluate compounds for antipsychotic-like CNS effects and a variety of other behavioral effects generally associated with CNS activity, the blockade of amphetamine-stimulated locomotion in rat, the protocol for the prepulse inhibition of acoustic startle model in rats, the inhibition of apomorphine-induced climbing behaviour and the inhibition of DOI-induced head twitches and scratches. A preferred animal model is the STOP−/− mice with synaptic defects and severe behavioral disorders described by Andrieux et al. (2002, Genes Dev., 16:2350-2364).

3. PP2A/Bγ-Related Biallelic Markers

The present invention is directed to the use of at least one PP2A/Bγ-related biallelic marker for diagnosing whether an individual suffers from or is at risk of suffering from a mental disorder; As used herein, the term “PP2A/Bγ-related biallelic marker” refers to a biallelic marker located in an exon of PPP2R2C, in an intron of PPP2R2C, or in the regulatory regions of PPP2R2C, PPP2R2C being the gene encoding the PP2A/Bγ subunit. KCNQ2-related biallelic markers encompass the biallelic markers shown in table 3A in Example 12.

In one embodiment, a single biallelic marker is used for diagnosing whether an individual suffers from or is at risk of suffering from a mental disorder by determining the genotype of an individual. In another embodiment, a combination of several biallelic markers may be used for diagnosing whether an individual suffers from or is at risk of suffering from a mental disorder by determining the haplotype of an individual. For example, a two-markers haplotype, a three-markers haplotype or a four-markers haplotype may be determined.

As used herein, the term “biallelic marker” refers to a polymorphism having two alleles at a fairly high frequency in the population, preferably a single nucleotide polymorphism. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). In the present specification, the term “biallelic marker” is used to refer both to the polymorphism and to the locus carrying the polymorphism.

As used herein, the term “genotype” refers to the identity of the alleles present in an individual or a sample. The term “genotype” preferably refers to the description of both copies of a single biallelic marker that are present in the genome of an individual. The individual is homozygous if the two alleles of the biallelic marker present in the genome are identical. The individual is heterozygous if the two alleles of the biallelic marker present in the genome are different.

The term “genotyping” a sample or an individual for a biallelic marker involves determining the specific alleles or the specific nucleotides carried by an individual at a biallelic marker.

As used herein, the term “haplotype” refers to a set of alleles of closely linked biallelic markers present on one chromosome and which tend to be inherited together.

Methods for determining the alleles, genotypes or haplotypes carried by an individual are well known by those of skill in the art and further detailed below.

In all aspect and embodiments, preferred “mental disorders” include bipolar disorder, schizophrenia and depression. Most preferred mental disorder is bipolar disorder.

In the context of the present invention, the individual is generally understood to be human.

In the frame of the present invention, four validated PP2A/Bγ-related biallelic markers are provided. These four markers, 99-24169/139, 24-257/320, 99-24175/218 and 24-247/216, are described in table 3A in Example 12. Thus a preferred embodiment is the use of a PP2A/Bγ-related biallelic marker selected from the group consisting of 99-24169/139, 24-257/320, 99-24175/218 and 24-247/216 for diagnosing whether an individual suffers from or is at risk of suffering from a mental disorder. The alternative alleles of these four biallelic markers are indicated in table 3A. Positions of these biallelic markers on PPP2R2C (corresponding to SEQ ID NO: 37) are also indicated in table 3B. Other preferred embodiments are directed to the use of biallelic markers complementary to 99-24169/139, 24-257/320, 99-24175/218 and 24-247/216, i.e., the corresponding alternative alleles that are located on the complementary strand of DNA.

As shown in table 5A in Example 15, association studies were performed for these four biallelic markers. All these four PP2A/Bγ-related biallelic markers were found to be bipolar disorder-associated markers. A preferred embodiment of the present invention is thus directed to the use of a PP2A/Bγ-related biallelic marker selected from the group consisting of 99-24169/139, 24-257/320, 99-24175/218, 24-247/216 and the complements thereof for diagnosing whether an individual suffers from or is at risk of suffering from a mental disorder.

The individual may be of any ethnic origin. In one embodiment the individual is an individual of Caucasian origin.

The risk alleles for biallelic markers 99-24169/139, 24-257/320 and 99-24175/218 are indicated in Example 15. As used herein, “risk allele” means that the probability of having bipolar disorder is higher for an individual carrying the risk allele of a biallelic marker than for an individual carrying the other allele. The risk allele for 99-24169/139, 24-257/320 and 99-24175/218 is “A”. Thus a preferred embodiment of the present invention is the use of any of biallelic markers 99-24169/139, 24-257/320, 99-24175/218 or the complement thereof for diagnosing whether an individual suffers from or is at risk of suffering from a mental disorder, wherein the presence of the allele “A” at any of biallelic markers biallelic markers 99-24169/139, 24-257/320 or 99-24175/218 is indicative of said individual suffering from or being at risk of suffering from said mental disorder.

The risk genotype for biallelic marker 99-24169/139 is indicated in table 6A in Example 15. As used herein, “risk genotype” means that-the probability of having bipolar disorder is higher for an individual carrying the risk genotype than for an individual carrying another genotype. The risk genotype for biallelic marker 99-24169/139 is “AA”. Thus a preferred embodiment of the present invention is the use of biallelic marker 99-24169/139 or the complement thereof for diagnosing whether an individual suffers from or is at risk of suffering from a mental disorder, wherein the presence of a genotype “AA” at biallelic marker 99-24169/139 is indicative of said individual suffering from or being at risk of suffering from said mental disorder.

A haplotype frequency analysis was carried out for the four PP2A/Bγ-related biallelic markers 99-24169/139, 24-257/320, 99-24175/218 and 24-247/216 (Example 15). More specifically, the risk haplotype for biallelic markers 99-24169/139 and 24-247/216 and for biallelic markers 24-257/320 and 99-24175/218 were determined. As used herein, “risk haplotype” means that the probability of having bipolar disorder is higher for an individual carrying the risk haplotype than for an individual carrying another haplotype. The risk haplotype for biallelic markers 99-24169/139 and 24-247/216 is “AG”. Thus a preferred embodiment of the present invention is the use of biallelic markers 99-24169/139 and 24-247/216 or the complement thereof for diagnosing whether an individual suffers from or is at risk of suffering from a mental disorder, wherein the presence a haplotype “AG” at biallelic markers 99-24169/139 and 24-247/216 is indicative of said individual suffering from or being at risk of suffering from said mental disorder. The risk haplotype for biallelic markers 24-257/320 and 99-24175/218 is “AA”. Thus another preferred embodiment of the present invention is the use of biallelic markers 24-257/320 and 99-24175/218 or the complement thereof for diagnosing whether an individual suffers from or is at risk of suffering from a mental disorder, wherein presence of a haplotype “AA” at biallelic markers 24-257/320 and 99-24175/218 is indicative of said individual suffering from or being at risk of suffering from said mental disorder.

In all aspects and embodiments of the present invention, the term “PP2A/Bγ-related biallelic markers” encompasses biallelic markers 99-24169/139, 24-257/320, 99-24175/218, 24-247/216 and the complements thereof. Preferred PP2A/Bγ-related biallelic markers are 99-24169/139, 24-257/320, 99-24175/218 and the complements thereof are. Biallelic marker 99-24169/139 and the complement thereof are most preferred PP2A/Bγ-related biallelic markers.

A further aspect of the present invention pertains to the use of at least one PP2A/Bγ-related biallelic marker for determining whether there is a significant association between said marker and a mental disorder. Such determination may for example be performed using methods described in examples 10 to 15 below, but using populations that are different from the UCL and the Labimo populations, e.g., populations having different ethnic origins. The determination may also be performed for a trait that is not bipolar disorder but, e.g., schizophrenia or another mood or psychotic disorder. In this aspect of the invention, the PP2A/Bγ-related biallelic marker may be selected from the group consisting of 99-24169/139, 24-257/320, 99-24175/218, 24-247/216 and the complements thereof. Alternatively, the PP2A/Bγ-related biallelic marker may be a marker that is not specifically disclosed in the present specification.

The present invention is further directed to a method of genotyping comprising the step of determining the identity of a nucleotide at a PP2A/Bγ-related biallelic marker or the complement thereof in a biological sample. Preferably, said biological sample is derived from a single subject. It is preferred that the identity of the nucleotides at said biallelic marker is determined for both copies of said biallelic marker present in said individual's genome. In a preferred embodiment, the identity of the nucleotide at said biallelic marker is determined by a microsequencing assay. Preferably, a portion of a sequence comprising the biallelic marker is amplified prior to the determination of the identity of the nucleotide. The amplification may preferably be performed by PCR. Such a method of genotyping may for example be performed, using any of the protocols described in examples 10 to 14 of the present specification. Further methods of genotyping are well known by those of skill in the art and any other known protocol may be used.

Methods well-known to those skilled in the art that may be used for genotyping in order to detect biallelic polymorphisms include methods such as, e.g., conventional dot blot analyzes, single strand conformational polymorphism analysis (SSCP) (Orita et al. (1989) Proc Natl Acad Sci USA 86:2766-2770), denaturing gradient gel electrophoresis (DGGE) (Borresen et al. (1988) Mutat Res. 202:77-83.), heteroduplex analysis (Lessa et al. (1993) Mol Ecol. 2:119-129), mismatch cleavage detection (Grompe et al. (1989) Proc Natl Acad Sci USA. 86:5888-5892). Another method for determining the identity of the nucleotide present at a particular polymorphic site employs a specialized exonuclease-resistant nucleotide derivative as described in U.S. Pat. No. 4,656,127. Oligonucleotide microarrays or solid-phase capturable dideoxynucleotides and mass spectrometry may also be used (Wen et al. (2003) World J Gastroenterol. 9:1342-1346; Kim et al. (2003) Anal Biochem. 316:251-258). Preferred methods involve directly determining the identity of the nucleotide present at a biallelic marker site by sequencing assay, microsequencing assay, enzyme-based mismatch detection assay, or hybridization assay.

As used herein, the term “biological sample” refers to a sample comprising nucleic acids. Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like.

Methods of genotyping find use in, e.g., in genotyping case-control populations in association studies as well as in genotyping individuals in the context of detection of alleles of biallelic markers which are known to be associated with a given trait. In the context of the present invention, a preferred trait is a mental disorder selected from the group of bipolar disorder, schizophrenia and depression, and most preferably bipolar disorder.

Methods of genotyping may be used not only for determining the genotype of an individual but also for determining the haplotype of an individual. When determining the haplotype of an individual, each single chromosome should be studied independently. Methods of determining the haplotype of an individual are well known in the art and include, e.g., asymmetric PCR amplification (Newton et al. (1989) Nucleic Acids Res. 17:2503-2516; Wu et al. (1989) Proc. Natl. Acad. Sci. USA. 86:2757-2760), isolation of single chromosome by limit dilution followed by PCR amplification (Ruano et al. (1990) Proc. Natl. Acad. Sci. USA. 87:6296-6300) and, for sufficiently close biallelic markers, double PCR amplification of specific alleles (Sarkar and Sommer, (1991) Biotechniques. 10:436-440).

Thus the present invention is further directed to the use of at least one PP2A/Bγ-related biallelic marker for determining the haplotype of an individual. For example, a method for determining a haplotype for a set of biallelic markers in an individual may comprise the steps of: a) genotyping said individual for at least one PP2A/Bγ-related biallelic marker, b) genotyping said individual for a second biallelic marker by determining the identity of the nucleotides at said second biallelic marker. Preferably, both markers are PP2A/Bγ-related biallelic markers. Methods of determining a haplotype for a combination of more than two biallelic markers comprising at least one PP2A/Bγ-related biallelic marker in an individual are also encompassed by the present invention. In such methods, step (b) is repeated for each of the additional markers of the combination. Such a combination may comprise, e.g., 3, 4 or 5 biallelic markers. These biallelic markers may all be PP2A/Bγ-related biallelic markers.

When estimating haplotype frequencies in a population, one may use methods without assigning haplotypes to each individual. Such methods use a statistical method of haplotype determination. Thus another aspect of the present invention encompasses methods of estimating the frequency of a haplotype for a set of biallelic markers in a population, comprising the steps of: a) genotyping each individual in said population for at least one PP2A/Bγ-related biallelic marker, b) genotyping each individual in said population for a second biallelic marker by determining the identity of the nucleotides at said second biallelic marker; and c) applying a haplotype determination method to the identities of the nucleotides determined in steps a) and b) to obtain an estimate of said frequency. Such a method may also be performed for a combination of more than 2 biallelic markers. Step (c) may be performed using any method known in the art to determine or to estimate the frequency of a haplotype in a population. Preferably, a method based on an expectation-maximization (EM) algorithm (Dempster et al. (1977) JRSSB, 39:1-38; Excoffier and Slatkin, (1995) Mol Biol Evol. 12:921-7) leading to maximum-likelihood estimates of haplotype frequencies under the assumption of Hardy-Weinberg proportions (random mating) is used for performing step (c).

A preferred aspect of the present invention is directed to a method of diagnosing a mental disorder in an individual comprising the step of genotyping at least one PP2A/Bγ-related biallelic marker using a method of genotyping comprising the step of determining the identity of a nucleotide at a PP2A/Bγ-related biallelic marker or the complement thereof in a biological sample derived from said individual. Such a diagnosing method may further comprise the step of correlating the result of the genotyping step with a risk of suffering from said mental disorder. Typically, the presence of the risk allele, risk genotype or risk haplotype of the genotyped PP2A/Bγ-related biallelic marker(s) is correlated with a risk of suffering from the mental disorder. The PP2A/Bγ-related biallelic marker may be selected from the group consisting of biallelic markers 99-24169/139, 24-2571320, 99-24175/218, 24-2471216 and the complements thereof. In one embodiment, the presence of a genotype “AA” at biallelic marker 99-24169/139 is indicative of a risk of suffering from said mental disorder. In another embodiment, the presence a haplotype “AG” at biallelic markers 24169/139 and 24-247/216 is indicative of a risk of suffering from said mental disorder. In still another embodiment, the presence of a haplotype “AA” at biallelic markers 24-257/320 and 99-24175/218 is indicative of a risk of suffering from said mental disorder.

In a further aspect, the present invention pertains to the use of a polynucleotide comprising a contiguous span of at least 12 nucleotides of SEQ ID NO: 37 or a polynucleotide complementary thereto in a microsequencing assay for determining the identity of the nucleotide at a PP2A/Bγ-related biallelic marker, wherein the 3′ end of said polynucleotide is located 1 nucleotide upstream of said PP2A/Bγ-related biallelic marker in said sequence. Such nucleotides may comprise a contiguous span of at least 12, 15, 18, 19 or 20 nucleotides of SEQ ID NO: 37 or a polynucleotide complementary thereto. They preferably comprise a contiguous span of about 19 nucleotides of SEQ ID NO: 37 or a polynucleotide complementary thereto. In one embodiment, said PP2A/Bγ-related biallelic marker is selected from the group consisting of 99-24169/139, 24-257/320, 99-24175/218, 24-247/216 and the complements thereof. In a preferred embodiment, the polynucleotide comprising a contiguous span of at least 12 nucleotides of SEQ ID NO: 37 or a polynucleotide complementary thereto is selected from the group consisting of the primers depicted in table 4A in Example 14.

### EXAMPLES

### Example 1

### Yeast Two-Hybrid Screening

1. Construction of pGBKT7-PPP2R2C

The full-length coding region of the PPP2R2C gene, which encodes the PP2A/Bγ subunit, was first amplified from a Human foetal brain cDNA library (Marathon-Ready cDNA, Clontech) with the two gene-specific primers of SEQ ID NO: 8 and of SEQ ID NO: 9. This first PCR product was then amplified with a new combination of primers of SEQ ID NO: 10 and of SEQ ID NO: 11. The amplified fragment encompassed nucleotides 52-1540 of the full-length cDNA, genbank accession number AF086924 extended, respectively, with EcoRI and BamHI cloning sites. The resulting 1503-bp fragment was digested with EcoRI and BamHI, purified and inserted into EcoRI and BamHI cloning sites of the pGBKT7 vector (Clontech).

2. The Yeast Two-Hybrid Screening

A yeast two-hybrid screening was performed to find polypeptides interacting with the PP2A/Bγ subunit. The Saccharomyces cerevisiae strain AH109 (MATa, trp1-901, leu2-3, 112, ura3-52, his3-200, gal4Δ, gal80Δ, LYS2:: GAL1_{UAS}-GAL1_{TATA}-HIS3, GAL2_{TATA}-ADE2, URA3:: MEL1_{UAS}-MEL1_{TATA}-lacZ) was transformed with the pGBKT7-PPP2R2C construction. A lithium acetate transformation procedure was done according to the manufacturer's instructions (Matchmaker Two-Hybrid system, Clontech). The MATa transformed cells expressing the bait were then mixed with a pretransformed Matchmaker Human brain cDNA library in the Y187 strain (MATα, ura3-52, his3-200, ade2-101, tip1-901, leu2-3, 112, gal4Δ, met⁻, gal80Δ, URA3:: GAL1_{UAS}-GAL1_{TATA}-lacZ). Three independent mating were performed with respectively 5.10⁶, 5.10⁶ and 2.10⁵ clones of the Human brain cDNA library. The resulting diploid cells able to grow on SD/-Leu/-Trp medium containing plates were further selected onto the medium-stringency SD/-Leu/-Trp/-His selective medium for the identification of bait-prey interactions. Positive colonies were then picked up and plated onto the high-stringency SD/-Leu/-Trp/-His/-Ade selective medium. Only cDNA of colonies able to grow at the same time on SD/-Leu/-Trp and SD/-Leu/-Trp/-His/-Ade media was retained for sequencing and further studies.

3. Results of the he Yeast Two-Hybrid Screening

494 clones were obtained, sequenced and analyzed. Among these clones, the 2E11 and 1D3 clones comprised partial cDNAs encoding a novel splice variant of the KCNQ2 potassium channel. 2E11 comprised a cDNA encoding amino acids 433 to 643 of SEQ ID NO: 2, and 1D3 comprised a cDNA encoding amino acids 454 to 643 of SEQ ID NO: 2. The full-length splice variants were cloned and sequenced as described in Example 2.

### Example 2

### Cloning of the Full-Length KCNQ2 Splice Variants

1. Cloning and Sequencing

Poly(A)+ mRNA from Human brain, thalamus (Clontech) were reversed transcribed (RT) using the murine Moloney leukemia virus reverse transcriptase (RT-PCR Advantage kit, Clontech) with a primer of SEQ ID NO: 12 hybridizing specifically with the novel splice variant cloned in 2E11. After a phenol-chloroform extraction and precipitation steps, the products obtained by the previous RT-PCR were directly PCR-amplified using the following gene-specific primers of SEQ ID NO: 13 and of SEQ ID NO: 14. The amplified fragment encompassed nucleotides 127-148 of the KCNQ2 full-length cDNA, genbank accession number AF033348. These primers were respectively extended with EcoRI and Bg/II cloning sites. The PCR products were digested with EcoRI and Bg/II restriction enzymes (New England Biolabs), purified and then ligated into the EcoRI and Bg/II cloning sites of the pCMV-Myc vector (Clontech). The two pCMV-Myc-3H9 and pCMV-Myc-3H2 clones were fully sequenced. The sequence of the insert in pCMV-Myc-3H2 comprises SEQ ID NO: 1, and the sequence of the insert in pCMV-Myc-3H9 comprises SEQ ID NO: 3.

Similarly, a cDNA was cloned from a poly(A)+ mRNA library from human foetal brain. One clone was obtained and fully sequenced. Its insert comprised SEQ ID NO: 5.

2. Description of the Novel Splice Variants

SEQ ID NO: 1 encodes the polypeptide of SEQ ID NO: 2 (KCNQ2-15bx). SEQ ID NO: 3 encodes the polypeptide of SEQ ID NO: 4 (KCNQ2-15by). SEQ ID NO: 5 encodes the polypeptide of SEQ ID NO: 6 (KCNQ2-15bz). SEQ ID NO: 7 corresponds to the full-length KCNQ2 polypeptide (KCNQ2-fl).

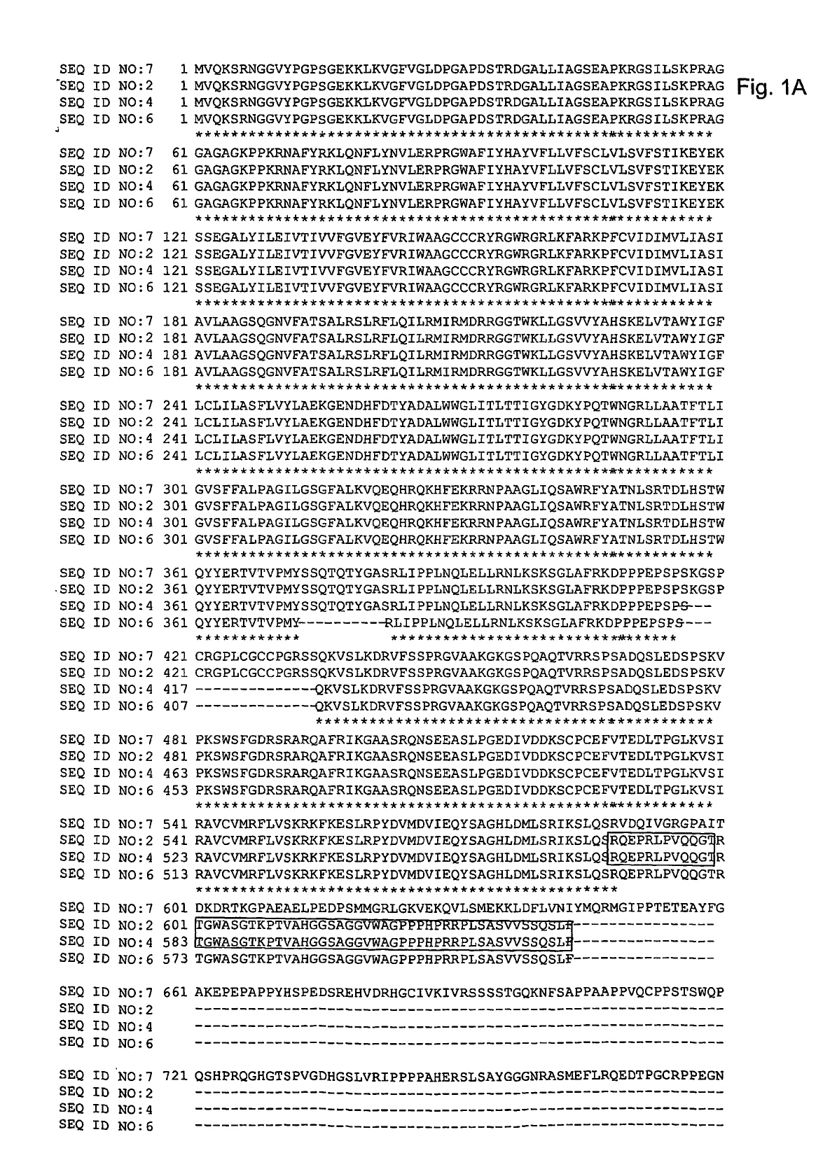

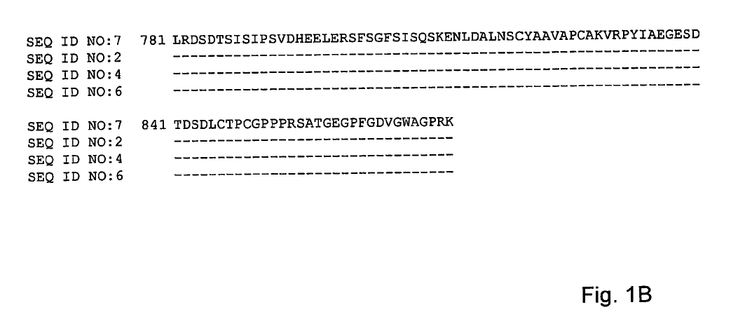

As shown on the alignment between SEQ ID NO: 7, SEQ ID NO: 2, SEQ ID NO: 4 and SEQ ID NO: 6 (FIG. 1), the three splice variants display a novel carboxyl-terminal extremity compared to KCNQ2. The 55 carboxyl-terminal amino acids of SEQ ID NO: 2, SEQ ID NO: 4 and SEQ ID NO: 6 are unique to these three splice variants. These 55 amino acids correspond to the amino acids at position 589 to 643 of SEQ ID NO: 2.

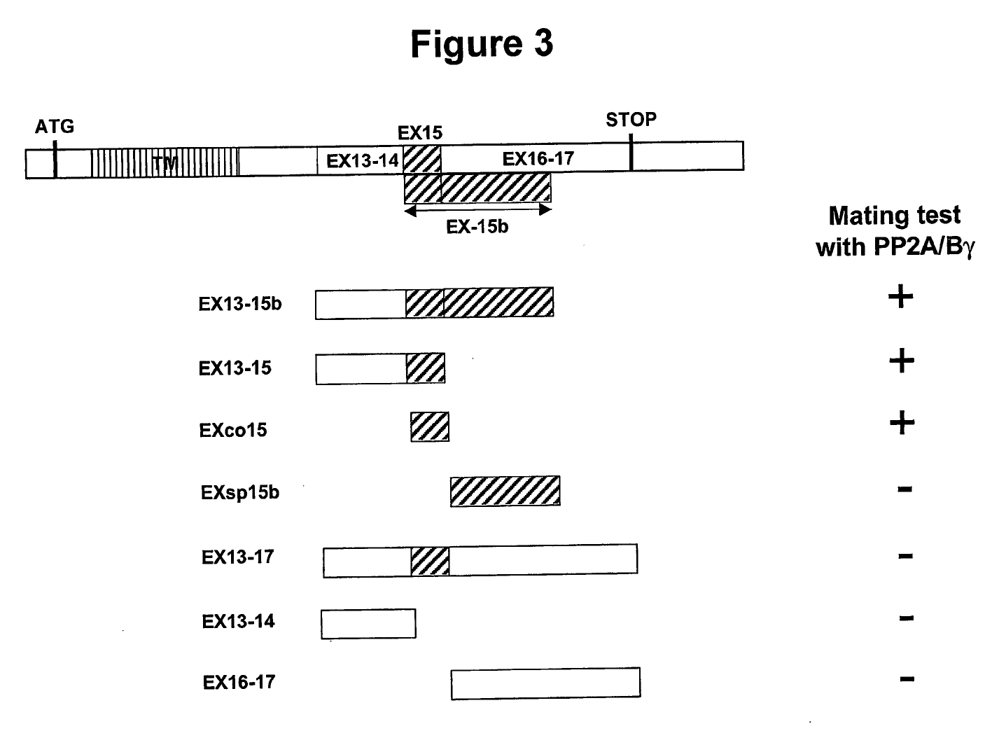

The genomic structure of the KCNQ2 gene is shown on FIG. 3 and in table 1. The KCNQ2 gene is comprised of 17 exons. None of the novel splice variants displays the exons corresponding to exons 15, 16 and 17 of the KCNQ2 gene. They all display a novel exon, exon 15b, which encodes the amino acids at position 545 to 643 of SEQ ID NO: 2. The 44 first amino acids encoded by exons 15 and 15b are identical (amino acids at position 545 to 588 of SEQ ID NO: 2). The 55 last amino acids encoded by exon 15b are unique to exon 15b (amino acids at position 589 to 643 of SEQ ID NO: 2). Furthermore, the novel splice variants do not display exons 16 and 17 of KCNQ2-fl. The most carboxyl-terminal exon of these splice variants is exon 15b. SEQ ID NO: 2 further comprises exon 1 to exon 14 of KCNQ2. Exon 12 of KCNQ2 is lacking in SEQ ID NO: 4. Exons 9 and 12 of KCNQ2 are lacking in SEQ ID NO: 6.

The insert of the 2E11 clone, which corresponds to a partial cDNA, comprises exons 13, 14 and 15b.

TABLE 1

| | | Encodes | | Encodes | | Encodes | Encodes |
|---|---|---|---|---|---|---|---|
| Exon | SEQ ID | AA of SEQ | SEQ ID | AA of SEQ | SEQ ID | AA of SEQ | AA of SEQ |
| No. | NO: 1 | ID NO: 2 | NO: 3 | ID NO: 4 | NO: 5 | ID NO: 6 | ID NO: 7 |
| 1 | 1-296 | 1-98 | 1-296 | 1-98 | 1-296 | 1-98 | 1-98 |
| 2 | 297-387 | 100-129 | 297-387 | 100-129 | 297-387 | 100-129 | 100-129 |
| 3 | 388-514 | 130-171 | 388-514 | 130-171 | 388-514 | 130-171 | 130-171 |
| 4 | 515-690 | 173-230 | 515-690 | 173-230 | 515-690 | 173-230 | 173-230 |
| 5 | 691-816 | 231-272 | 691-816 | 231-272 | 691-816 | 231-272 | 231-272 |
| 6 | 817-927 | 273-309 | 817-927 | 273-309 | 817-927 | 273-309 | 273-309 |
| 7 | 928-1023 | 310-341 | 928-1023 | 310-341 | 928-1023 | 310-341 | 310-341 |
| 8 | 1024-1118 | 342-372 | 1024-1118 | 342-372 | 1024-1118 | 342-372 | 342-372 |
| 9 | 1119-1148 | 374-382 | 1119-1148 | 374-382 | / | / | 374-382 |
| 10 | 1149-1217 | 384-405 | 1149-1217 | 384-405 | 1119-1187 | 374-395 | 384-405 |
| 11 | 1218-1247 | 407-415 | 1218-1247 | 407-415 | 1188-1217 | 397-405 | 407-415 |
| 12 | 1248-1301 | 417-433 | / | / | / | / | 417-433 |
| 13 | 1302-1525 | 435-508 | 1248-1471 | 417-490 | 1218-1441 | 407-480 | 435-508 |
| 14 | 1526-1631 | 510-543 | 1472-1577 | 492-525 | 1442-1547 | 482-515 | 510-543 |
| 15 | / | / | / | / | / | / | 545-587 |
| 15b | 1632-1929 | 545-643 | 1578-1875 | 527-625 | 1548-1845 | 517-615 | / |
| 16 | / | / | / | / | / | / | 588-629 |
| 17 | / | / | / | / | / | / | 630-872 |

### Example 3

### Yeast Mating Test

1. Construction of Vectors

1.1. EX13-17, Which Comprises Exons 13, 14, 15, 16 and 17.

The pGADT7-EX13-17 plasmid was constructed as follows: a 1414-bp fragment was first PCR-amplified from a Human total brain cDNA library (Marathon-Ready cDNA, Clontech) with two gene-specific primers of SEQ ID NO: 15 and of SEQ ID NO: 16. This first PCR product was then amplified with a second set of gene-specific primers of SEQ ID NO: 17 and 5′ of SEQ ID NO: 18. These primers are extended, respectively, with EcoRI and BamHI cloning sites. After digestion with EcoRI and BamHI restriction enzymes, the 1338-bp purified fragment was ligated to the same cloning sites of pGADT7 (Clontech).

1.2. EX13-15, Which Comprises Exons 13, 14 and 15.

The pGADT7-EX13-15 plasmid was obtained as follows: a 484-bp fragment was PCR-amplified with primers of SEQ ID NO: 19 and of SEQ ID NO: 20, which are respectively extended with EcoRI and BamHI cloning sites, from the first PCR product of the pGADT7-EX13-17 construction. The resulting fragment was then digested with EcoRI and BamHI, purified, and ligated to the same cloning sites of pGADT7 (Clontech).

1.3. EX16-17, Which Comprises Exons 16 and 17.

The pGADT7-EX16,17 plasmid was obtained as follows: a 883-bp fragment was PCR-amplified with primers of SEQ ID NO: 21 and of SEQ ID NO: 22, which are respectively extended with EcoRI and BamHI cloning sites, from the first PCR product of the pGADT7-EX13-17 construction. The resulting fragment was then digested with EcoRI and BamHI, purified, and ligated to the same cloning sites of pGADT7 (Clontech).

1.4. EXsp15b, Which Comprises the Region Unique to Exon 15b.

The pGADT7-EXsp15b plasmid was constructed as follows: a 400-bp fragment was PCR-amplified with a primer of SEQ ID NO: 23 extended with EcoRI cloning site, and with a primer of SEQ ID NO: 24 from the pACT2-2E11 plasmid (see example 1). The resulting fragment was then digested with EcoRI and XhoI, purified, and ligated to the same cloning sites of pGADT7 (Clontech).

1.5. EXco15, Which Comprises the Region Common to Exon 15 and Exon 15b.

The pGADT7-EXco15 domain plasmid was constructed as follows: a 146-bp fragment was PCR-amplified with primers of SEQ ID NO: 25 and of SEQ ID NO: 26, which are respectively extended with EcoRI and BamHI cloning sites, from the pACT2-2E11 plasmid. The resulting fragment was then digested with EcoRI and BamHI, purified, and ligated to the same cloning sites of pGADT7 (Clontech).

1.6. EX13-14, Which Comprises Exons 13 and 14.

The pGADT7-EX13-14 plasmid was constructed as follows: a 300-bp fragment was PCR-amplified with primers of SEQ ID NO: 27 and of SEQ ID NO: 28, which are respectively extended with EcoRI and BamHI cloning sites, from the pACT2-2E11 plasmid. The resulting fragment was then digested with EcoRI and BamHI, purified, and ligated to the same cloning sites of pGADT7 (Clontech).

2. Protocol of the Yeast Mating Test

Yeast mating tests were performed to map the interaction domains between the different partners. The chosen Saccharomyces cerevisiae mating partner strains (AH109 and Y184) were transformed separately with the plasmids to be tested in combination with the plasmid of interest. The lithium acetate transformation procedure was done according to the manufacturer's instructions (Matchmaker Two-Hybrid system, Clontech). Transformants were selected on the appropriate SD dropout medium (Clontech). One fresh colony of each type to use was picked from the working stock plates and both placed in one 1.5 ml microcentrifuge tube containing 0.5 ml of YPD medium (Clontech). Cells were then incubated for 24 hr at 30° C. with shaking at 200 rpm. 100 μl of a 1:100 dilution of the mating culture were then spread on the appropriate SD medium: SD/-Leu/-Trp, and SD/-Leu/-Trp/-His/-Ade. After 7 to 15 days of growth on selective medium positive colonies were counted.

3. Results of the Direct Mating Tests Between KCNQ2 Polypeptides and PP2A/Bγ

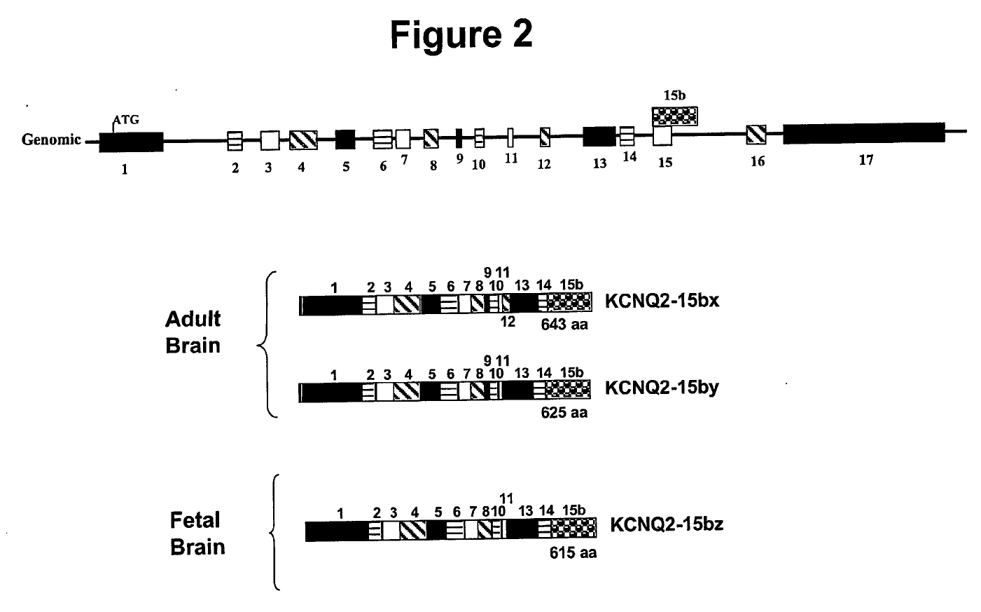

Mating tests between each of the above constructions and the pGBKT7-PPP2R2C construction described in example 1 were performed. The results are shown on FIG. 2. The sign “+” indicates that colonies grew, thus indicating that the tested polypeptide is capable of interacting with PP2A/Bγ. The sign “−” indicates that no colony grew, thus indicating that the tested polypeptide does not interact with PP2A/Bγ.

EX13-17, EX16-17, EX13-14 and EXsp15b do not interact with PP2A/Bγ. EX13-15b, EX13-15 and EXco15 interact with PP2A/Bγ. Since EX13-15b interacts with PP2A/Bγ, this shows that KCNQ2-15b polypeptides are capable of interacting with PP2A/Bγ. Since EX13-15b, EX13-15 and EXco15 but not EXsp15b interact with PP2A/Bγ, the common region between exon 15 and exon 15b plays a role in this interaction. Furthermore, since EX13-17 does not interact with PP2A/Bγ, the fact that exon 15 or that exon 15b is located at the most carboxyl extremity of the KCNQ2 polypeptide is of importance for efficient interaction with PP2A/Bγ.

4. Results of the Direct Mating Tests Between Different KCNQ2 Polypeptides

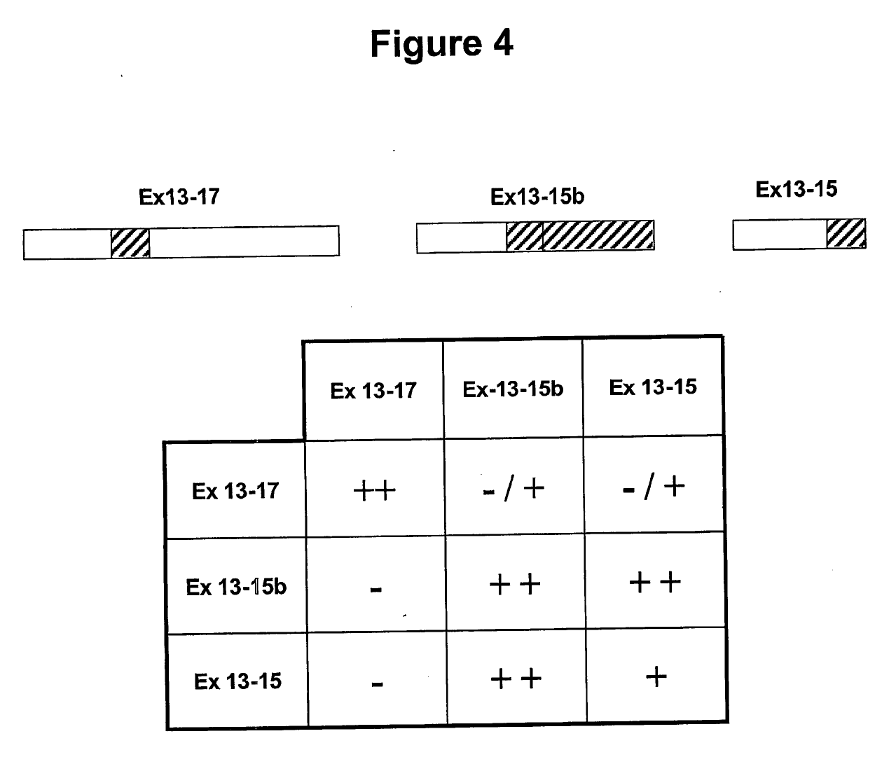

Mating tests between the different above constructions were performed, and the results are shown on FIG. 4. 4 mating tests were performed for each pair of constructs and the results are shown on FIG. 3. The sign “++” indicates that all 4 colonies grew. The sign “+” indicates that 3 colonies out of 4 grew. The sign “±” indicates that 1 colony out of 4 grew. The sign “−” indicates that no colony grew.

This experiment shows that KCNQ2-15b polypeptides can associate and form homodimers. KCNQ2-15b polypeptides can also associate and form heterodimers with KCNQ2 polypeptides comprising exon 15 at their carboxyl-terminal extremity. KCNQ2-15b polypeptides only associate poorly with KCNQ2-fl polypeptides.

### Example 4

### Expression and Purification of Glutathione S-Transferase Fusion Proteins

1. Construction of Plasmids

1.1. pGBKT7-2E11

The pACT2-2E11 plasmid rescued from yeast two-hybrid screening was digested with EcoRI and BglII and the resulting 687-bp fragment inserted after purification into EcoRI and BamHI cloning sites of the pGBKT7 vector (Clontech).

2.2. pGEX-2TK-2E11

A partial cDNA of the KCNQ2 splice variants was PCR-amplified from the pACT2-2E11 plasmid rescued from yeast two-hybrid screening using a gene-specific primer of SEQ ID NO: 29 and a primer in the pACT2 vector of SEQ ID NO: 30. These primers were respectively extended with BamHI and EcoRI cloning sites. The 892-bp PCR product was digested with BamHI and EcoRI, purified and inserted into BamHI and EcoRI sites of pGEX-2TK vector (Amersham Pharmacia Biotech). The pACT2 plasmid used for this construction was recovered from diploid cells as follows: a fresh colony of diploid cells was inoculated into 5 ml of SD/-Leu/-Trp (Clontech) and let to grow overnight at 30° C. with shacking at 200-250 rpm. Cells corresponding to 2 ml of the overnight culture were spun down by centrifuging at 4300 rpm for 10 min. The pellet was resuspended in 100 μl of zymolyase (1 U/μl) (Seikagaku Corporation) and incubated 1 hr at 30° C. Then 100 μl of a proteinase K mix (100 mM NaCl, 10 mM Tris-HCl pH [pH 8.0], 25 mM EDTA, 0.5% SDS, 0.1 mg/ml proteinase K) were added for 2.5 hr at 40° C. DNA was extracted by two successive phenol:chloroform steps and precipitated with 0.3 M sodium acetate and 2.5 volumes of ethanol. DH10B ElectroMAX competent cells (Invitrogen) were transformed with DNA and selected on agar plates supplemented with 120 μg/ml Ampicillin. The protein encoded by pGEX-2TK-2E11 was named GST-2E11.

1.3. pGEX-2TK-PPP2R2C

A 1485-bp fragment of PPP2R2C encompassing nucleotides 55-1540 of the full-length cDNA of PP2A/Bγ (genbank accession number AF086924) was PCR-amplified from the pGBKT7-PPP2R2C plasmid using gene-specific primers of SEQ ID NO: 31 and of SEQ ID NO: 32, which are respectively extended with BamHI and EcoRI cloning sites. The fragment was digested by BamHI and EcoRI, purified and ligated to the same cloning sites of pGEX-2TK vector (Amersham Pharmacia Biotech). The protein encoded by pGEX-2TK-2E11 is named GST-PPP2R2C.

1.4. pGEX-2TK-KCNQ2-Cter

A 1393-bp fragment of a KCNQ2-fl encompassing nucleotides 1544-2924 of the full-length cDNA (genbank accession number AF033348) was PCR-amplified from the pCMV-HA-KCNQ2-iso1 construction using gene-specific primers: of SEQ ID NO: 33 and of SEQ ID NO: 34, which are respectively extended with XhoI and EcoRI cloning sites. This PCR product was digested with XhoI and EcoRI, purified and substituted at the same sites for a 767-bp XhoI-EcoRI fragment of the pGEX-2TK-2E11 plasmid. The pCMV-HA-KCNQ2-iso1 plasmid used for the construction of pGEX-2TK-KCNQ2-Cter was obtained as follows: the full-length coding region for KCNQ2-fl (encompassing nucleotides 126-2924 of the full-length cDNA, genbank accession number AF033348) was first amplified from a Human brain cDNA library (Marathon-Ready cDNA, Clontech) using gene specific primers of SEQ ID NO: 35 and of SEQ ID NO: 36, which are respectively extended with EcoRI and BglII cloning sites. The PCR product was digested with EcoRI and BglII, purified and ligated to the same cloning sites of the pCMV-HA vector (Clontech). The protein encoded by pGEX-2TK-2E11 is named GST-KCNQ2-Cter.

2. Expression and Purification

Glutathione S-transferase fusion protein expression and purification by adapting the method described by Kaelin et al. (1991, Cell, 64:521-532). Overnight cultures of MAX Efficiency DH5αF'IQ competents cells (Invitrogen) transformed with either the pGEX2TK plasmid or the pGEX2TK-2E11, pGEX2TK-KCNQ2-Cter, and pGEX2TK-PPP2R2C recombinants were diluted 1:10 in LB medium containing ampicillin (100 μg/ml) and incubated for 1 hr at 37° C. Isopropyl-β-D-thiogalactopyranoside (IPTG, Promega) was then added to a final concentration of 0.1 mM and bacteria let to grow for 3 additional hours at 37° C. For fusion proteins recovery using the glutathione-Sepharose 4B beads (Amersham Biosciences), bacterial cultures were pelleted by centrifugation at 5000×g for 15 min at 4° C. and resuspended in 1/10 vol NETN (20 mM Tris-HCl [pH 8.0], 120 mM NaCl, 1 mM EDTA, 0.5% Nonidet P-40) supplemented with 1 mM phenylmethylsulfonyl fluoride (PMSF, Sigma) and one tablet of protease inhibitors cocktail (Complete™ mini, Roche) for 7 ml of buffer. The bacteria were then lysed on ice by mild sonication and centrifuged at 10,000×g for 10 min at 4° C. Aliquots (1 ml) of bacterial clear lysates were then rocked for 1 hr at 4° C. with 50 μl of glutathione-Sepharose 4B beads, which had been previously washed four times in NETN containing 1% Albumin Bovine (BSA fraction V, Sigma) and resuspended (final concentration 1:1 [v/v]) in NETN. The glutathione-Sepharose 4B beads were then washed three times with NETN. For recovery of the bound recombinants proteins, beads were washed two more times with 100 mM Tris-HCl [pH 8.0], 120 mM NaCl and elution was performed in the same buffer containing 20 mM glutathione (Sigma). Quantification of the eluted fusion proteins was performed by the standard Bradford's method (Biorad Protein Assay).

### Example 5

### In vitro Labeling of the GST Fusion Proteins

Beads with bound GST fusion proteins corresponding to 1 ml of bacterial clear lysate were washed three times in NETN and one time with HMK buffer without DTT (20 mM Tris-HCl [pH 7.5], 120 mM NaCl, 12 mM MgCl₂). Beads were then resuspended in 30 μl of reaction mix (3 μl of 10× HMK Buffer with 20 mM DTT, 10 units of Protein Kinase A Catalytic Subunit [PKA from bovine heart, 250 units/vial, Sigma] in 40 mM DTT, 2 μl of [³²P]-γATP 6000 Ci/mMole and 24 μl of distilled water) and incubated at 4° C. for 30 min. During incubation beads were resuspended time to time by flicking. Reaction was stopped by adding 1 ml of HMK stop buffer (10 mM Sodium Phosphate [pH 8.0], 10 mM Sodium Pyrophosphate, 10 mM EDTA, 1 mg/ml BSA) and beads washed five times with NETN buffer. Elution of radiolabeled fusion proteins was carried out with 1 ml of freshly prepared 20 mM glutathione in 100 mM Tris-HCl [pH 8.0], 120 mM NaCl as previously described.

### Example 6

### Solid Phase Overlay Assay

1. Protocol of the Solid Phase Overlay Assay

Solid phase overlay assays were performed by adapting the method described by Kaelin and collaborators (Kaelin et al., 1992, Cell, 70:351-364). 100 ng, 10 ng and 0.1 ng of GST and GST-2E11 recombinant proteins were resolved by 9% SDS-PAGE and were transferred by electroblotting onto nitrocellulose membrane (nitrocellulose transfer membrane Protran BA 83, Schleicher and Schuell). The membrane were then blocked in HBB buffer (25 mM Hepes-KOH [pH 7.7], 25 mM NaCl, 5 mM MgCl₂) with 5% (w/v) non-fat dry milk, 1 mM DTT, 0.05% Nonidet P-40 for 1 hr at room temperature. The binding reaction was carried out at room temperature in Hyb75 buffer (20 mM Hepes [pH 7.7], 75 mM KCl, 2.5 mM MgCl₂, 0.1 mM EDTA, 0.05% Nonidet P40) with 1% (w/v) non-fat dry milk, 1 mM DTT, 1 mM PMSF and 3.5 10⁵ dpm of a [³²P]-γATP GST-PPP2R2C radiolabeled recombinant protein used as a probe. After 4.5 hr of incubation, the membrane was washed with Hyb75 buffer, 1 mM DTT, 1% (w/v) non-fat dry milk three times for 15 min at room temperature. The blots were analyzed by autoradiography.

2. Results

This experiment was performed to validate the interaction between KCNQ-15b polypeptides and PP2A/Bγ. In this experiment, the PP2A/Bγ subunit was radiolabeled but not the proteins present on the nitrocellulose membrane. Thus, a signal appears when visualized by autoradiography only if the loaded protein interacts with PP2A/Bγ. GST-2E11 corresponds to a fusion protein between a KCNQ2-15b polypeptide comprising exons 13, 14 and 15b and GST. GST corresponds to the negative control. In the three lines loaded with the GST-2E11 recombinant protein, bands located at a position corresponding to a protein of a size of about 45 kD appeared. This corresponds to the protein size expected for the GST-2E11 protein. Furthermore, the intensity of the bands was proportional to the quantity of loaded GST-2E11. Thus GST-2E11 interacts with PP2A/Bγ. In the three lines loaded with the GST protein, no band appeared, showing that PP2A/Bγ doe s not interact with the GST protein. Thus the interaction between PP2A/Bγ and the GST-2E11 fusion protein is due to the part of the protein encoding 2E11 and not to the part of the protein encoding GST. This experiment indicates that KCNQ-15b polypeptides can interact with PP2A/Bγ in vitro. Furthermore, this shows that KCNQ-15b polypeptides can interact with PP2A/Bγ without a third binding partner, a hypothesis that can not be excluded by a yeast-two hybrid assay.

### Example 7

### In vitro Phosphorylation Assay With Recombinant GSK-3β Kinase and In vitro dephosphorylation with HTB-14 Whole Cell Extracts.

1. Phosphorylation Assays

Phosphorylation assays were performed to determine whether the phsophorylation state of KCNQ2-15b is modulated by GSK3β, a kinase that plays an important role in the central nervous system by regulating various cytoskeletal processes through its effects on MAP1B, tau and synapsin 1. GSK3β is known to be inhibited by two mood stabilizing agents used in treatment of bipolar disorder, lithium and valporate.

1. 1. Protocol

Expression and purification of the GST-2E11 fusion protein were performed as described above. Beads with bound fusion protein corresponding to 1 ml of bacterial clear lysate were washed three times in NETN and one time with HMK buffer without DTT (20 mM Tris-HCl [pH 7.5], 120 mM NaCl, 12 mM MgCl₂). Beads were resuspended in 240 μl of reaction mix (24 μl of 10× HMK Buffer with 20 mM DTT, 40 units of Protein Kinase A Catalytic Subunit [PKA from bovine heart, 250 units/vial, Sigma] in 40 mM DTT, 5 μl of 24 mM ATP and 207 μl of distilled water) and incubated for 30 min at room temperature. Beads were then washed three times in NETN buffer and one time in GSK -3β reaction buffer (20 mM Tris-HCl [pH 7.5], 10 mM MgCl₂, 5 mM DTT) (New England Biolabs). Beads were then resuspended in 50 μl of reaction mix (5 μl of 10× GSK-3β reaction buffer, 1 μl of [³²P]γATP 10 mCi/ml, 50 U of recombinant GSK-3β[New England Biolabs], and distilled water for a final volume of 50 μl) and incubated at room temperature for 30 min. After three washes in NETN buffer, phosphorylated proteins were boiled in 2× Sample Buffer (125 mM Tris-HCl [pH 6.8], 4% SDS, 20% glycerol, 1.4 M β-Mercapto ethanol), resolved by 10% SDS-PAGE, and visualized by autoradiography.

1.2. Results

In this phosphorylation assay, non-radiolabeled polypeptides to be tested are incubated in the presence of GSK-3β, PKA and radioactive ATP. The proteins are then resolved by a 10% SDS-PAGE migration and visualized by autoradiography. A signal is visualized by autography only if the protein to be tested is phosphorylated by GSK -3β and PKA during incubation. In the line loaded with the GST-2E11 protein, which corresponds to the fusion protein between a KCNQ2-15b polypeptide comprising exons 13, 14 and 15b and the GST polypeptide, a band located at a position corresponding to a protein of a size of about 45 kD did appear. This is the size expected for the GST-2E11 protein. Thus the GST-2E11 protein is phosphorylated by GSK-3β and PKA in vitro. Three experiments corresponding to negative controls were performed in parallel. One experiment was performed without adding the GSK-3β kinase during incubation, one was performed without adding the PKA kinase during incubation, and one was performed with a GST protein instead of a GST-2E11 protein. No bands appeared in the three lines corresponding to the negative controls. Accordingly, this experiment shows that KCNQ2-15b polypeptides are synergistically phosphorylated by the GSK-3β and PKA kinases in vitro.

This result was confirmed by a competition experiment in which CREB phosphopeptides, which are known to be phosphorylated by GSK-3β and PKA, were added during incubation. In this competition experiment, 5 μg of CREB phosphopeptides (New England Biolabs) was added to the kination mix. A band did still appear at a position corresponding to the size of GST-2E11, but the intensity of the band was very significantly lower.

The influence of LiCl on the phosphorylation state of GST-2E11 was further studied by adding LiCl to the kination mix at a final concentration of 0, 8.3, 25, 75 and 225 mM respectively. The intensity of the band appearing at a position of about 45 kD decreased in the presence of LiCl, and the intensity of the signal was negatively correlated with the concentration of LiCl added to the kination mix. In the presence of about 50 mM LiCl, the phosphorylation state of GST-2E11 was reduced by 50%. This shows that LiCl, a well-known mood-stabilizing agent used in the treatment of bipolar disorder, inhibits phosphorylation of KCNQ2-15b polypeptides in vitro.

2. Dephosphorylation Assays

Dephosphorylation assays were performed to determine whether the phophorylation state of KCNQ2-15b polypeptides is modulated by PP2A.

2.1. Protocol

In vitro phosphorylated GST-2E11 fusion protein was incubated at room temperature for 30 min with 500 μg of whole cell extracts of Human glioblastoma, astrocytoma cell line (ATCC number: HTB-14) with or without 400 μM of the PP2A phosphatase inhibitor okadaic acid (Sigma). HTB-14 whole cell extracts were prepared as follow: cells were washed three times with ice-cold TBS buffer (10 mM Tris-HCl [pH 8.0], 120 mM NaCl) and lysed at 4° C. for 30 min in EBC buffer (50 mM Tris-HCl [pH 8.0], 120 mM NaCl, 0.5% Nonidet P-40). Then the lysate was centrifugated for 10 min at 13.000×g at 4° C. to pellet cell debris. Proteins present in the supernatant were quantified by the standard Bradford's method (Bio-Rad Protein Assay). The proteins were then resolved by 10% SDS-PAGE, and visualized by autoradiography.

2.2. Results

The phosphorylated radiolabeled GST-2E11 proteins obtained from the previous assay were incubated in the presence of HTB-14 cell extracts containing the PP2A phosphatase to determine whether PP2A is capable of dephosphorylating GST-2E11 proteins. In this experiment, a protein that is dephosphorylated by PP2A is not radioactive after incubation in the presence of HTB-14 cell extracts any more. Thus dephosphorylation of the GST-2E11 protein is monitored by disappearance of the signal visualized by autoradiography. One line of the 10% SDS -PAGE gel was loaded with phosphorylated GST-2E11 fusion proteins incubated in the absence of HTB-14 cell extracts, as reference for the intensity of the band appearing for phosphorylated GST-2E11 proteins. In the line loaded with GST-2E11 fusion proteins incubated in the presence of HTB-14 cell extracts, the band had an extremely weaker intensity. Thus GST-2E11 fusion proteins are dephosphorylated when incubated in the presence of HTB-14 cell extracts. When the GST-2E11 fusion protein was incubated in the presence of HTB-14 cell extracts and okadaic acid, a known PP2A phosphatase inhibitor, the intensity of the band was only slightly weaker than the intensity of the band corresponding to phosphorylated GST-2E11. Thus the PP2A phosphatase is responsible of the dephosphorylation observed for GST-2E11 fusion proteins incubated in the presence of HTB-14 cell extracts. Accordingly, this experiment shows that KCNQ-15b polypeptides are dephosphorylated by the PP2A phosphatase in vitro.

### Example 8

### Cell Culture, Transfection, Immunoprecipitation and Western Blot Analysis

1. Cell Cultures

HEK293-H cells (Gibco Invitrogen Corporation) were grown in DMEM medium (Gibco Invitrogen Corporation) supplemented with 0.1 mM Non-Essential Amino Acids and 10% Fetal Bovine Serum (Gibco Invitrogen Corporation), and transiently transfected with 20 μg of the pCMV-Myc-3H9 or pCMV-Myc-3H2 plasmids per 60 mm dish using the Invitrogen calcium phosphate transfection kit and protocols. 48 hr after transfection cells were washed three times with ice-cold phosphate buffer (PBS, Gibco Invitrogen Corporation), scraped and solubilized for 2 hr at 4° C. in solubilization buffer containing 150 mM NaCl, 5 mM EDTA, 1% Triton X-100, 0.1% sodium deoxycholate, 10 mM Tris-HCl [pH 8.0] and supplemented with protease inhibitors (1 mM phenylmethylsulfonyl fluoride, one tablet of Complete™ mini protease inhibitors cocktail [Roche]) and phosphatase inhibitors (1 mM Na₃VO₄ and 1 mM NaF). The lysate was then centrifugated for 10 min at 13.000×g at 4° C. to pellet cell debris. Proteins present in the supernatant were quantified by the standard Bradford's method (Bio-Rad Protein Assay).

2. Immunoprecipitation

500 μg (final volume: 500 μl) of the clear cell lysate were incubated for 2 hr at 4° C. with 1 μl of rabbit preimmune serum and 50 μl of protein A Sepharose CL-4B beads (Amersham Pharmacia Biotech) saturated with 1% Albumin Bovine (BSA fraction V, Sigma). Depleted supernatants were then incubated overnight at 4° C. with 1 μg of anti-Myc monoclonal antibody (Myc-Tag 9B11 monoclonal antibody, Cell Signaling). Protein A Sepharose CL-4B beads saturated with 1% Albumin Bovine were then added and the mixture incubated at 4° C. for 2 addtional hours. After five washes with ice-cold solubilization buffer immuno-complexes were boiled in 2× Sample Buffer (125 mM Tris-HCl [pH 6.8], 4% SDS, 20% glycerol, 1.4 M β-Mercapto ethanol), resolved by 8% SDS-PAGE and subjected to

3. Western Blot

Proteins were transferred onto nitrocellulose membrane (nitrocellulose transfer membrane Protran BA 83, Schleicher and Schuell) using Towbin buffer (Towbin et al., 1979, PNAS, 76:4350-4354) and an electrotransfer device. After transfer, membranes were blocked, in 5% non-fat dried milk in TBST (10 mM Tris-HCl [pH 8.0], 150 mM NaCl, 0.05% Tween 20) supplemented with sodium azide (0.1%) for 2 hr. and then incubated for 16 hr at room temperature with the anti-Myc monoclonal antibody (Myc-Tag 9B11 monoclonal antibody, Cell Signaling) diluted 1:1000 in the same buffer. After several washes with TBST, the blot was incubated with a horseradish peroxidase-conjugated secondary antibody (Anti-mouse IgG, Fab specific, peroxidase conjugate, Sigma) diluted 1:5000 and developed using ECL Western blotting detection reagents (Amersham Biosciences).

### Example 9:

### Electrophysiological Analysis

1. Protocols

1.1. cDNA injection in Xenopus laevis Oocytes

The animal was anesthetized and pieces of the ovary were surgically removed and individual oocytes were dissected away in a saline solution (ND96) containinng 96 mM NaCl, 2 mM KCl, 2 mM CaCl2, 2 mM MgCl2 and 5 mM HEPES at pH 7.4. Stage V and VI oocytes were treated at room temperanre for 2 h with collagenase type 1A (1 mg/ml) in the presence of 0.2 mg/ml trypsin inhibitor in saline solution to discard follicular cells. The concentrations were determined by measuring the absorbance at 260 nm. DNA corresponding to KCNQ2, 3H2 and 3H9 K+ channels were subcloned in PEKO vector in order to generate the respective cRNAS. cRNA concentrations were measured by absorbance at 260 nM. cRNA solutions were injected (about 50 nL/oocyte) using a pressure microinjector (Inject+matic, Genève). Oocytes were then kept for 2-6 days in ND96 solution supplemented wirn 50 U/mL penicillin and 50 U/mL streptomycin.

1.2. Electrophysiological Measurements

In a 0.3 rmL perfusion chamber, a single oocyte was impaled with two standard glass microelectrode (0.5-2 Mohm resistance) filled with 3M KCl and maintained under voltage clamp using a Dagan TEV2OO amplifier system, USA. Electrical stimulations, data acquisition and analyses were performed using pClamp software (Axon Instruments, USA). Current to voltage relationships were obtained applying incremental depolarizing voltage steps (10 mV increment) from a holding potential of −80 mV (equilibrium potential for K+ ions) Repolarizations to −60 mV allowed K+ channel deactivation measurements from the “tail currents”.

2. Results

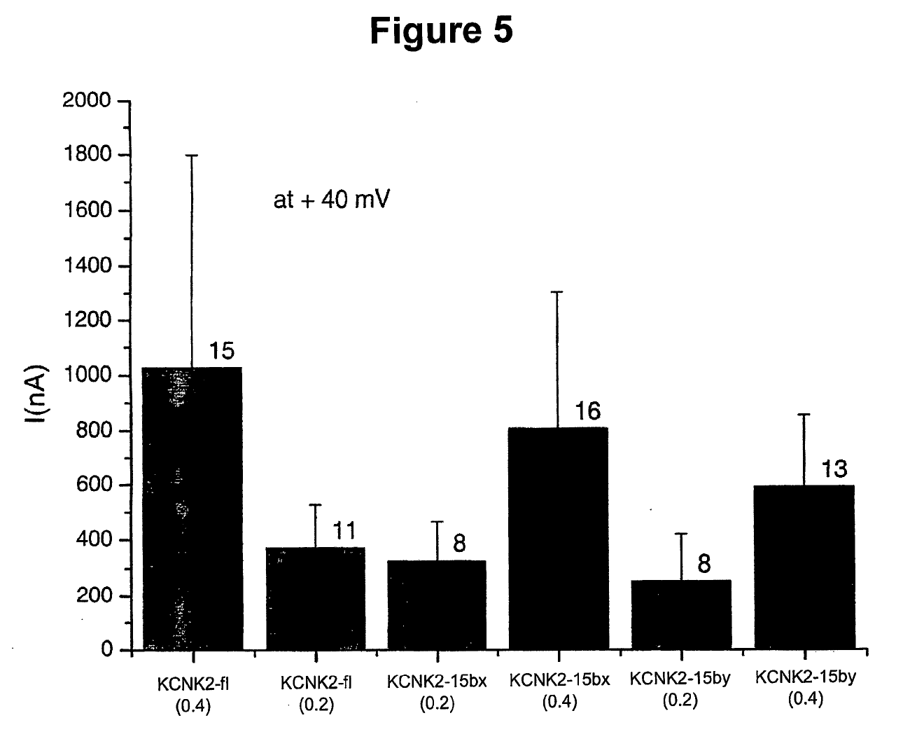

The activity of KCNQ2-15bx and of KCNQ-15by homotetrameric potassium channels was tested and compared to the activity of KCNQ2-fl homotetrameric potassium channels. 0.2 ng or 0.4 ng of DNA were injected to the oocytes. The results are shown on FIGS. 5, on which the intensity of the M-current generated by the potassium channels is indicated. An intensity of about 1 μA is found for the current generated by a of KCNQ2-fl homotetrameric potassium channel when 0.4 ng of DNA is injected. This value is similar to the value reported by scientific literature. A KCNQ2-15bx homotetrameric potassium channel yields a current of about 800 nA when 0.4 ng of DNA is injected, and a KCNQ2-15by homotetrameric potassium channel yields a courant of about 700 nA when 0.4 ng of DNA is injected. Thus the KCNQ2-15bx and KCNQ-15by splice variants can associate as functional homomeric potassium channels in vivo.

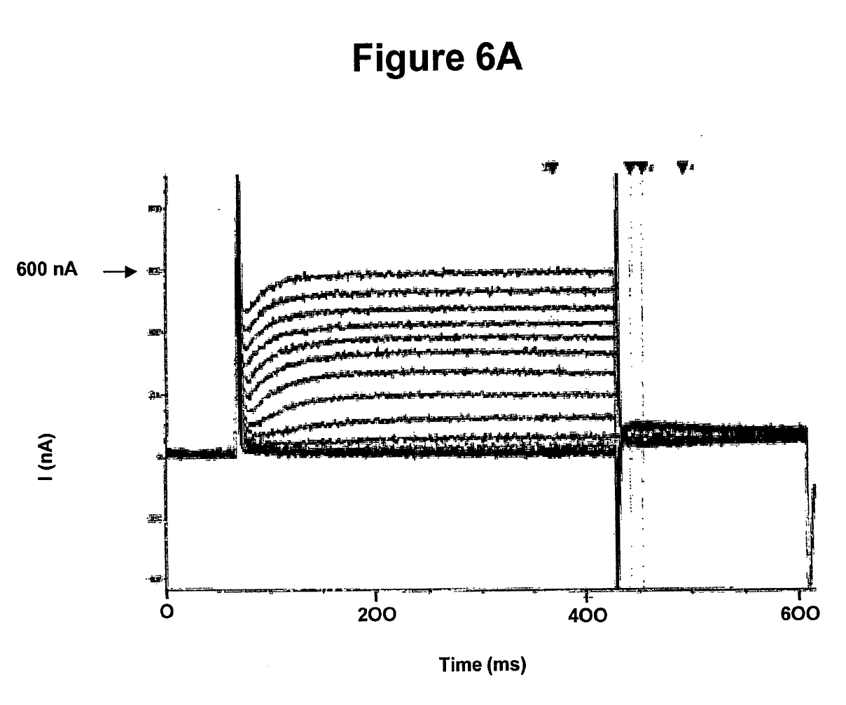

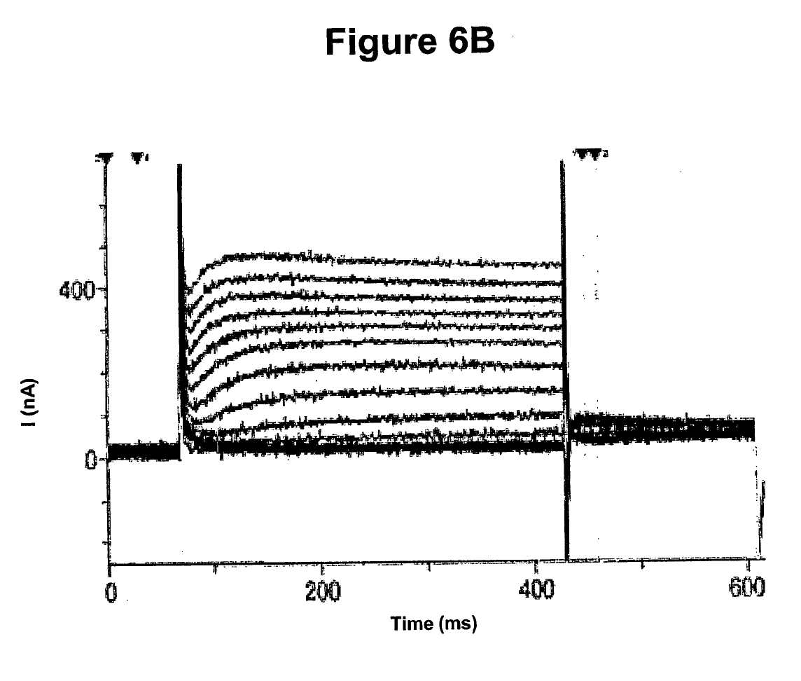

FIG. 6A and FIG. 6B show the voltage clamp traces corresponding to the currents generated at different voltages by KCNQ2-15bx (FIG. 6A) and by KCNQ2-15by (FIG. 6B) homotetrameric potassium channels. The slow activation that is observed on the traces is a characteristic feature of members of the KCNQ potassium channel family.

### Example 10

### Collection of DNA Samples From Affected and Non-Affected Individuals

Donors were unrelated and healthy. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH7.6; 5 mM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:
3 ml TE 10-2 (Tris-HCl 10 mM, EDTA 2 mM)/NaCl 0 4 M
200 μl SDS 10%
500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA). To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 11

### Amplification of Genomic DNA By PCR

The amplification of specific genomic sequences of the DNA samples of Example 10 was carried out on the pool of DNA obtained previously. In addition, 50 individual samples were similarly amplified.

PCR assays were performed using the following protocol:

| | | |
|---|---|---|
| Final volume | 25 | μl |
| DNA | 2 | ng/μl |
| MgCl₂ | 2 | mM |
| dNTP (each) | 200 | μM |
| primer (each) | 2.9 | ng/μl |
| Ampli Taq Gold DNA polymerase | 0.05 | unit/μl |

| | |
|---|---|
| PCR buffer (10× = 0.1 M | 1× |
| TrisHCl pH 8.3 0.5 M KCl) | |

Each pair of first primers was designed using the sequence information of genomic DNA sequences and the OSP software (Hillier & Green, 1991).

Primers Biallelic Markers Located in PPP2R2C

The genomic sequence of PPP2R2C that is shown as SEQ ID NO: 37 was constructed upon bioinformatic analysis based on (i) BAC clones constructed at Genset S. A.; (ii) BAC clones corresponding to EMBL Accesion Nos. AC114815.5, AC004599.6, AC122939.3 and AC004689.5; and (iii) RefseqN Accession No. NT_{—}006051. The polymorphisms were identified as described in examples 12 and 13, and validated as described in example 14.

Biallelic Markers Located in the KCNQ2 Gene

The biallelic markers located in the KCNQ2 gene were found using data provided by Celera. Each of these markers were further validated as described in example 14.

Table 2A indicates the position on SEQ ID NO: 37 of pairs of primers that were used to amplify specific regions of PPP2R2C. Table 2B indicates the position of the primers on SEQ ID Nos 42 to 47, which were used to amplify specific regions of KCNQ2. The orientation of the primer is indicated in the third column. The sign (+1) indicates that the sequence of the primer is identical to the corresponding region of SEQ ID Nos. 37 and 42 to 47. The sign (−1) indicates that the sequence of the primer is complementary to the corresponding region of SEQ ID Nos. 37 and 42 to 47.

TABLE 2A
Primer location in PPP2R2C

| Name of the | Position on | |
|---|---|---|
| amplified region | SEQ ID NO: 37 | Orientation |
| 24-257 | 109495 to 109512 | (+1) |
| | 109963 to 109982 | (−1) |
| 99-24169 | 83709 to 83729 | (+1) |
| | 84146 to 84164 | (−1) |
| 99-24175 | 117228 to 117248 | (+1) |
| | 117659 to 117677 | (−1) |
| 24-247 | 99290 to 99309 | (+1) |
| | 99719 to 99738 | (−1) |

TABLE 2B
Primer location in the KCNQ2 gene

| Name of the | | | |
|---|---|---|---|
| amplified region | SEQ ID No. | Position | Orientation |
| 30-4 | SEQ ID NO: 42 | 244 to 263 | (+1) |
| | | 324 to 343 | (−1) |
| 30-2 | SEQ ID NO: 43 | 240 to 258 | (+1) |
| | | 319 to 338 | (−1) |
| 30-17 | SEQ ID NO: 44 | 265 to 284 | (+1) |
| | | 345 to 364 | (−1) |
| 30-7 | SEQ ID NO: 45 | 272 to 291 | (+1) |
| | | 315 to 333 | (−1) |
| 30-84 | SEQ ID NO: 46 | 265 to 284 | (+1) |
| | | 334 to 353 | (−1) |
| 30-15 | SEQ ID NO: 47 | 248 to 267 | (+1) |
| | | 312 to 331 | (−1) |

Preferably, the primers contained a common oligonucleotide tail upstream of the specific bases targeted for amplification which was useful for sequencing.

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 12

### Identification of Biallelic Markers from Amplified Genomic DNA

The sequencing of the amplified DNA obtained in Example 11 was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version)).

The sequence data were further evaluated to detect the presence of biallelic markers within the amplified fragments. The polymorphism search was based on the presence of superimposed peaks in the electrophoresis pattern resulting from different bases occurring at the same position as described previously.

The locations of the biallelic markers detected in the fragments of amplification are as shown below in Tables 3A and 3B.

TABLE 3A
Biallelic Markers in the PPP2R2C gene

| amplified | | | polymorphism | | BM position on |
|---|---|---|---|---|---|
| region | BM name | Strand | All 1 | All 2 | SEQ ID NO: 37 |
| 24-257 | 24-257/320 | (−) | A | G | 109663 |
| 99-24169 | 99-24169/139 | (−) | A | G | 84026 |
| 99-24175 | 99-24175/218 | (−) | A | G | 117460 |
| 24-247 | 24-247/216 | (+) | A | G | 99505 |

TABLE 3B
Biallelic Markers in the KCNQ2 gene

| amplified | | | polymorphism | | SEQ ID | BM position on indicated |
|---|---|---|---|---|---|---|
| region | BM name | Strand | All 1 | All 2 | No. | SEQ ID No. |
| 30-4 | 30-4/58 | (+) | A | G | SEQ ID NO: 42 | 301 |
| 30-2 | 30-2/62 | (+) | A | G | SEQ ID NO: 43 | 301 |
| 30-17 | 30-17/37 | (+) | A | G | SEQ ID NO: 44 | 301 |
| 30-7 | 30-7/30 | (+) | C | T | SEQ ID NO: 45 | 301 |
| 30-84 | 30-84/37 | (+) | A | G | SEQ ID NO: 46 | 301 |
| 30-15 | 30-15/54 | (+) | A | C | SEQ ID NO: 47 | 301 |

BM refers to “biallelic marker”. All 1 and All 2 refer respectively to all ele 1 and allele 2 of the biallelic marker. The (+) or (−) sign in the column “strand of BM” indicates the strand on which the indicated alternative alleles are found. SEQ ID Nos. 37 and 42 to 47 correspond to strands (+). As a matter of example, the biallelic marker 24-257/320 corresponds to a polymorphism “a or g” at position 109663 on strand (−). Thus the nucleotide at position 109663 of SEQ ID NO: 37 will be “y”, which corresponds to “t or c” according to the standard PCT nomenclature. The biallelic marker 24-247/216 corresponds to a polymorphism “a or g” at position 99505 on strand (+). Thus the nucleotide at position 99505 of SEQ ID NO: 37 will be “r”, which corresponds to “a or g” according to the standard PCT nomenclature.

### Example 13

### Identification of Polymorphisms by Comparison of Genomic DNA from Overlapping BACs

Genomic DNA from multiple BAC clones derived from the same DNA donor sample and overlapping in regions of genomic DNA of SEQ ID NO: 37 was sequenced. Sequencing was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version)).

### Example 14

### Validation of The Polymorphisms Through Microsequencing

The biallelic markers identified in Examples 12 and 13 were further confirmed and their respective frequencies were determined through microsequencing. Microsequencing was carried out for each individual DNA sample described in Example 11.

Amplification from genomic DNA of individuals was performed by PCR as described above for the detection of the biallelic markers with the same set of PCR primers described in tables 1A and 1B.

The preferred primers used in microsequencing were about 19 nucleotides in length and hybridized just upstream of the considered polymorphic base. According to the invention, the primers used for microsequencing are detailed in tables 4A and 4B.

TABLE 4A
Primers in the PPP2R2C gene

| | | Orien- | | |
|---|---|---|---|---|
| | | tation | Position of | |
| amplified | Marker | of the | the primer on | SEQ ID No. |
| region | name | primer | SEQ ID NO: 37 | of the primer |
| 24-257 | 24-257/320 | (+1) | 109644 to 109662 | SEQ ID NO: 40 |
| 99-24169 | 99-24169/ | (+1) | 84007 to 84025 | SEQ ID NO: 39 |
| | 139 | | | |
| 99-24175 | 99-24175/ | (+1) | 117441 to 117459 | SEQ ID NO: 41 |
| | 218 | | | |
| 24-247 | 24-247/216 | (+1) | 99486 to 99504 | — |

TABLE 4B
Primers in the KCNQ2 gene

| | | | | Position of |
|---|---|---|---|---|
| | | Orientation | | the primer on |
| amplified | Marker | of the | | indicated |
| region | name | primer | SEQ ID No. | SEQ ID No. |
| 30-4 | 30-4/58 | (−1) | SEQ ID NO: 42 | 302 to 319 |
| | | | | (primer B18) |
| 30-4 | 30-4/58 | (+1) | SEQ ID NO: 42 | 282 to 300 |
| | | | | (primer A19) |
| 30-2 | 30-2/62 | (−1) | SEQ ID NO: 43 | 302 to 320 |
| 30-17 | 30-17/37 | (−1) | SEQ ID NO: 44 | 302 to 324 |
| 30-7 | 30-7/30 | (+1) | SEQ ID NO: 45 | 280 to 300 |
| 30-84 | 30-84/37 | (−1) | SEQ ID NO: 46 | 302 to 318 |
| 30-15 | 30-15/54 | (−1) | SEQ ID NO: 47 | 302 to 323 |

As for the primers in tables 2A and 2B, the sign (+1) in the column “orientation” indicates that the sequence of the primer is identical to the corresponding region of SEQ ID Nos. 37 and 42 to 47, and the sign (−1) indicates that the sequence of the primer is complementary to the corresponding region of SEQ ID Nos. 37 and 42 to 47.

The microsequencing reaction performed as follows. After purification of the amplification products, the microsequencing reaction mixture was prepared by adding, in a 20 μl final volume: 10 pmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or heterozygous type based on the height ratio.

### Example 15

### Association Study Between Bipolar Disorder and the Biallelic Markers of the Invention

5.1. Collection of DNA Samples From Affected and Non-Affected Individuals

The association studies were performed on two different populations. One collection of samples was provided by Hospital Pinero, Buenos-Aires, Argentina (the “Labimo” collection). The other collection of samples was provided by the University College of London (the “UCL” collection). Both collections are constituted by individuals that are affected or not by bipolar disease.

A) The Labimo Collection

a) Affected Population

206 DNA samples from patients suffering from bipolar disorder (cases) were collected for genotyping analysis.

All patients fulfilled DSM-IV and ICD-10 criteria for bipolar type I (ICD-10: F30.x, F31.x) or bipolar type II (ICD-10: F31.8). All patients were of Caucasian ethnic origin up to the 2^{nd} generation.

All potential patients suffering from a medical disorder or from a drug abuse were excluded. According to DSM-IV criteria, 115 cases were classified as bipolar type I, 69 were bipolar type II, 22 were unclassified, and information concerning the type of bipolar disease was lacking in 20 cases (8.5%)

The main phenotypic data of the cases were as follows:
Mean age at first symptoms: 25.6 years (SD, 11; range, 8-58)
Mean age at inclusion: 43.3 years (SD, 13.8; range, 17-76)
Gender: 142 females and 84 males (ratio, 1.7)
Ethnic origin: 213 were European Caucasian, 7 were non-European Caucasians, and information was lacking in 6 cases (2.5%)
Family history of bipolar disease was found in 18.5%, whereas schizophrenia was found in 0.9%.

b) Unaffected Population

201 DNA samples from individuals not suffering from bipolar disorder (controls) were collected for genotyping analysis.

All controls were individuals lacking personal or familial history of psychiatric disease.

The main phenotypic data of the controls were as follows:
Mean age: 43.8 years (SD, 12; range, 21-72)
Gender: 118 females and 83 males (ratio, 1.4)

180 controls were European Caucasian, and 21 had mixed ethnic origin

c) Cases and Control Populations Selected for the Association Study

The case control populations were matched for ethnicity and sex which resulted in 159 cases and 159 control individuals. Among the cases, 96 cases suffered from type I bipolar disease, 56 cases suffered from type II bipolar disease, and 7 cases suffered from an undetermined type of bipolar disease. 33.8% of the cases were males. The mean age of the cases was of 43 and the median age was of 44. 41.4% of the controls were males. The mean age of the controls was of 44 and the median age was of 46.

The presence of population structure can result in spurious association, which is an association between phenotypes and markers that is not linked to any causative loci but due to a different ethnic origin. The Fst test is a general statistical tool for analyzing variances and that can be used to verify that a collection is homogeneous, i.e., that found associations are not linked to the structure of the population. The Fst value is calculated using random markers that are (i) unlinked and (ii) not associated with the trait to be studied. An Fst value close to 0 indicates that the collection is homogeneous and that any significant associations that are found are due to the trait under investigation (see, e.g., Bruce S. Weir, Genetic Data Analysis II, Edition Sinauer, San Francisco and Hartl and Clark, Populations genetics, Edition Sinauer, San Francisco). 66 random markers that were (i) unlinked and (ii) not associated with bipolar disorder were used to calculate the Fst value. An Fst value of 1.68e-01 was found for the found in the Labimo collection, indicating that this collection is homogeneous.

B) The UCL Collection

a) Affected Population

315 samples from patients suffering from bipolar disorder (cases) were collected for genotyping analysis.

All patients fulfilled DSM-IV criteria for bipolar type I (ICD-10: F30.x, F31.x) or bipolar type II (ICD-10: F31.8). All patients were unrelated individuals of Caucasian origins from the British Isles (including English, Welsh, Scottish and Irish) up to the 2^{nd} generation.

b) Unaffected Population

300 samples from unaffected control individuals (not suffering from bipolar disorder) were collected for genotyping analysis.

All control individuals showed (i) absence of personal history of psychiatric disease; and (ii) absence of familial history of psychiatric disease in first-degree relatives. All controls individuals of Caucasian origins from the British Isles (including English, Welsh, Scottish and Irish) up to the 2^{nd} generation.

c) Cases and Control Populations Selected for the Association Study

The population retained for the study was composed of 315 case s and 295 controls. Among the cases, 256 cases suffered from type I bipolar disease, 26 cases suffered from type II bipolar disease, and 33 cases suffered from an undetermined type of bipolar disease. About 36% of the cases were males. The mean age of the cases was of 46 and the median age was of 46. 48% of the controls were males. The mean age of the controls was of 37 and the median age was of 32.

59 random markers that were (i) unlinked; and (ii) not associated with bipolar disorder were used to calculate the Fst value. A Fst value of 3.41e-01 was found for the UCL collection, indicating that this collection is homogeneous.

5.2. Association Studies

A) Genotyping of Affected and Control Individuals

The general strategy to perform the association studies was to individually scan the DNA samples from all individuals in each of the populations described above in order to establish the allele frequencies of biallelic markers, and among them the biallelic markers of the invention, in the diploid genome of the tested individuals belonging to each of these populations.

Frequencies of every biallelic marker in each population (cases and controls) were determined by performing microsequencing reactions on amplified fragments obtained by genomic PCR performed on the DNA samples from each individual. Genomic PCR and microsequencing were performed as detailed above in Examples 11 to 13 using the described PCR primers and microsequencing primers.

B) Single Biallelic Marker Frequency Analysis

The difference between the allelic frequencies in the unaffected population and in the population affected by bipolar disorder was calculated for all five markers located in the KCNQ2 gene, and for all four markers located in the PPP2R2C gene. The allelic frequency of markers between cases and controls were investigated using the Pearson Chi squared test for allelic frequency and genotypic frequency distributions. A significant difference between observed and expected alleles/genotypes of a specific marker between case and control populations implies an association between the gene harboring this particular biallelic marker and bipolar disease. Both allelic and genotypic p-values were calculated for all markers. The p-values in tables 5A and 5B indicate the probability of no association between a biallelic marker and bipolar disorder considering the frequency. A p-value under 5e-02 indicates a significant association between the biallelic marker and bipolar disorder.

Odds ratio determination is a way of comparing the probability of having the disease when carrying a given allele versus when not carrying the allele. An odds ratio higher than 1 indicates that the probability of having bipolar disease is higher when carrying one of the alternative alleles, haplotypes or genotypes than when carrying the other ones. The odds ratio allows the identification of the “risk” allele, haplotype or genotype for an associated biallelic marker. The genotypic odds ratio was calculated for one biallelic marker located in PPP2R2C and for two markers located in the KCNQ2 gene (tables 6A and 6B).

TABLE 5A
p-values for biallelic markers located in PPP2R2C

| | Location in | | Chosen | All. Freq | All. Odds | Allelic | Genotypic |
|---|---|---|---|---|---|---|---|
| Marker Name | PPP2R2C | Collection | allele | Diff. | Ratio | p-value | p-value |
| 99-24169/139 | Intron 1d | UCL | A | 0.095 | 1.733 | 2.19e−04 | 3.61e−04 |
| | | Labimo | A | 0.002 | 1.012 | 9.46e−01 | 5.98e−01 |
| 24-247/216 | intron 4 | UCL | G | 0.047 | 1.275 | 7.75e−02 | 2.29e−02 |
| | | Labimo | G | 0.024 | 1.125 | 4.86e−01 | 7.65e−01 |
| 24-257/320 | Intron 5 | UCL | A | 0.018 | 1.079 | 5.52e−01 | 8.22e−01 |
| | | Labimo | A | 0.102 | 1.557 | 4.04e−03 | 1.19e−02 |
| 99-24175/218 | Intron 5 | UCL | G | 0.035 | 1.162 | 2.62e−01 | 3.99e−03 |
| | | Labimo | A | 0.096 | 1.546 | 6.69e−03 | 2.34e−02 |

TABLE 5B
p-values for biallelic markers in the KCNQ2 gene

| | Location in the | | Chosen | All. Freq | All. Odds | Allelic | Genotypic |
|---|---|---|---|---|---|---|---|
| Marker Name | KCNQ2 gene | Collection | allele | Diff. | Ratio | p-value | p-value |
| 30-4/58 | 5′ of the gene | UCL | — | — | — | — | — |
| | | Labimo | G | 0.03 | 1.24 | 3.03e−01 | 5.85e−01 |
| 30-2/62 | intron 1 | UCL | A | 0.05 | 1.23 | 7.76e−02 | 5.20e−03 |
| | | Labimo | A | 0.03 | 1.13 | 4.42e−01 | 1.15e−01 |
| 30-17/37 | intron 4 | UCL | A | 0.01 | 1.03 | 7.77e−01 | 9.12e−01 |
| | | Labimo | G | 0.03 | 1.13 | 4.70e−01 | 7.10e−01 |
| 30-7/30 | intron 12 | UCL | C | 0.05 | 1.21 | 1.05e−01 | 3.02e−02 |
| | | Labimo | C | 0.02 | 1.06 | 7.03e−01 | 5.32e−01 |
| 30-84/37 | 3′ of gene | UCL | A | 0.02 | 1.20 | 3.06e−01 | 3.69e−01 |
| | | Labimo | — | — | — | — | — |
| 30-15/54 | 3′ of gene | UCL | A | 0.01 | 1.06 | 6.92e−01 | 7.68e−01 |
| | | Labimo | — | — | — | — | — |

TABLE 6A
genotypic odds ratios for a biallelic marker located in PPP2R2C

| | | | odds | |
|---|---|---|---|---|
| Biallelic marker | collection | genotype | ratio | p-value |
| 99-24169/139 | UCL | AA vs GG | 1.9 | 8.50e−02 |
| | | AA vs AG | 2.06 | 7.20e−05 |
| | | AA vs (AG + GG) | 2.04 | 4.60e−05 |

TABLE 6B
genotypic odds ratios for biallelic
markers located in the KCNQ2 gene

| | | | odds | |
|---|---|---|---|---|
| Biallelic marker | collection | genotype | ratio | p-value |
| 30-2/62 | UCL | (AG + GG) vs AA | 1.05 | 4.60E−01 |
| | | AG vs AA | 1.28 | 1.70E−01 |
| | | AA vs GG | 1.51 | 8.00E−02 |
| | | AG vs (GG + AA) | 1.62 | 3.00e−03 |
| | | (AG + AA) vs GG | 1.82 | 1.50e−03 |
| 30-7/30 | UCL | (CC + CT) vs TT | 1.04 | 4.40E−01 |
| | | TT vs CT | 1.14 | 2.90E−01 |
| | | (CC + TT) vs CT | 1.37 | 3.80e−02 |
| | | CC vs TT | 1.58 | 3.80e−02 |
| | | CC vs (TT + CT) | 1.71 | 7.006−03 |

Biallelic Markers in PPP2R2C

Thus the four studied biallelic markers located in the PPP2R2C gene are found to be significantly associated with bipolar disease. More specifically, 99-24169/139 is found to be highly associated with bipolar disorder in the UCL collection (significant allelic and genotypic p-values). 24-2571320 and 99-24175/218 are highly associated with bipolar disorder in the Labimo collection (significant allelic p-values). In addition, 99-24175/218 is also associated with bipolar disorder in the UCL collection (significant genotypic p-value). 24-2471216 is associated with bipolar disorder in the UCL collection (significant genotypic p-value).

The risk allele for the 99-24169/139 biallelic marker is “A”. The risk alleles for the 24-257/320 biallelic marker and for the 99-24175/218 biallelic marker are also “A”.

The risk genotype for the 99-24169/139 biallelic marker is “AA”. Thus an individual carrying the genotype “AA” at biallelic marker 99-24169/13 is at risk of developing bipolar disorder.

Biallelic Markers in the KCNQ2 Gene

Two biallelic markers located in the KCNQ2 gene, 30-2/62 and 30-7/30, are significantly associated with bipolar disease. More specifically, 30-2/62 is found to be highly associated with bipolar disorder in the UCL collection (significant allelic and genotypic p-values). 30-7/30 is associated with bipolar disorder in the UCL collection (significant genotypic p-value).

The risk genotype for 30-2/62 is “AG”. The risk genotype for 30-7/30 is “CC”. Thus individuals carrying the genotype “AG” at biallelic marker 30-2/62 and individuals carrying the genotype “CC” at biallelic marker 30-7/30 are at risk of developing bipolar disorder.

The association results of the single biallelic marker frequency analysis show that both the PPP2R2C gene and the KCNQ2 gene are associated with bipolar disorder. Accordingly, deregulation and/or dysfunction of KCNQ2 polypeptides and PP2A phosphatases comprising the PP2A/Bγ regulatory subunit contribute to the onset and to the development of bipolar disease.

C) Haplotype Frequency Analysis

One way of increasing the statistical power of individual markers is to perform haplotype association analysis. The analysis of haplotype frequencies cannot readily be derived from observed genotypic data. The EM (Expectation-Maximization) algorithm (Excoffier L & Slatkin M, 1995) allows the estimation of haplotypes for the population under investigation. Haplotype frequency estimations were performed by applying the OMNIBUS likelihood ratio test (PCT publication WO 01/091026)

The haplotype analysis was performed for two sets of markers located in PPP2R2C. The haplotype analysis for biallelic markers 24-257/320 and 99-24175/218 was performed in the Labimo collection. The haplotype analysis for biallelic markers 99-24169/139 and 24-247/216 was performed in the UCL collection. The results are shown in tables 7 (p-values) and 7B (odds ratios).

TABLE 7A

| | | | | Ave | SD | Max | |
|---|---|---|---|---|---|---|---|
| markers | Samples | Haplotype | Chi-S | Chi-S | Chi-S | Chi-S | p-value |
| 24-257/320 | | AA | 7.78 | 0.96 | 1.34 | 14.02 | 3.9e−03 |
| and | Labimo | AG | 0.02 | 1.02 | 1.40 | 11.19 | 8.79e−01 |
| 99-24175/218 | | GA | 0.14 | 0.96 | 1.35 | 11.62 | 6.77e−01 |
| | | GG | 7.35 | 0.98 | 1.35 | 14.31 | 5.5e−03 |
| 99-24169/139 | UCL | AA | 1.49641 | 1.03501 | 1.46687 | 14.67815 | 2.28e−01 |
| and | | AG | 5.19606 | 1.0854 | 1.52336 | 14.42852 | 2.73e−02 |
| 24-247/216 | | GA | 13.91081 | 1.29859 | 1.81182 | 16.01507 | 5e−04 |
| | | GG | 0.42926 | 1.57482 | 2.19562 | 23.4845 | 6.03e−01 |

TABLE 7B

| markers | haplotype | overall | cases | controls | odds ratio |
|---|---|---|---|---|---|
| 24-257/320 | AA | 60.9% | 65.9% | 55.5% | 1.55 |
| and | AG | 2.8% | 2.7% | 2.9% | 0.93 |
| 99-24175/218 | GA | 5.9% | 5.5% | 6.2% | 0.88 |
| | GG | 30.4% | 25.8% | 35.4% | 0.64 |
| 99-24169/139 | AA | 60.0% | 62.0% | 58.2% | 1.17 |
| and | AG | 17.4% | 20.0% | 14.5% | 1.47 |
| 24-247/216 | GA | 13.6% | 9.5% | 17.6% | 0.49 |
| | GG | 8.9% | 8.5% | 9.7% | 0.86 |

The risk haplotype for 24-257/320 and 99-24175/218 is “AA”. The risk haplotype for 99-24169/139 and 24-247/216 is “AG”. Thus an individual carrying the haplotype “AA” at biallelic markers 24-257/320 and 99-24175/218 is at risk of developing bipolar disorder, and an individual carrying the haplotype “AG” at biallelic markers 99-24169/139 and 24-247/216 is also at risk of developing bipolar disorder.

### REFERENCES

1. Agostinis et al. (1996) Eur J Biochem. 236:548-557
2. Altschul et al. (1990) J Mol Biol, 215:403-410
3. Altschul et al., (1997) Nucleic Acids Res., 25:389402
4. Andrieux et al. (2002) Genes Dev., 16 :2350-2364
5. Borresen et al. (1988) Mutat Res. 202:77-83
6. Dempster et al. (1977) JRSSB, 39:1-38
7. Detera-Wadleigh et al. (1999) Proc Natl Acad Sci USA, A96(10):5604-5609
8. Devereux et al. (1984) Nucleic Acids Res., 12:387-395
9. Elbashir et al. (2001) Genes Dev. 15:188-200
10. Ellington and Szostak (1990) Nature 346:818-822
11. Excoffier and Slatkin, (1995) Mol Biol Evol. 12:921-7
12. Grantham (1974) Science, 185:862-864
13. Grompe et al. (1989) Proc Natl Acad Sci USA. 86:5888-5892
14. Hu et al. (2000) Genomics., 67:83-86
15. Janssens and Goris (2001) Biochem J. 353:417-439
16. Kaelin et al. (1991) Cell, 64:521-532
17. Kaelin et al. (1992) Cell, 70:351-364
18. Kamibayashi et al. (1994) J Biol Chem. 269:20139-20148
19. Kim et al. (2003) Anal Biochem. 316:251-258
20. Lessa et al. (1993) Mol Ecol. 2:119-129
21. Newton et al. (1989) Nucleic Acids Res. 17:2503-2516
22. Orita et al. (1989) Proc Natl Acad Sci USA 86:2766-2770
23. Pan et al. (2001) J. Physiol., 531:347-358
24. Pearson (1990) Methods in Enzymology, 183:63-99
25. Pearson and Lipman (1988) Proc Nat Acad Sci USA, 85:2444-2448
26. Price et al. (2000) Biochemistry. 39:11312-11318
27. Ruano et al. (1990) Proc. Natl. Acad. Sci. USA. 87:6296-6300
28. Sarkar and Sommer, (1991) Biotechniques. 10:436-440
29. Schwake et al. (2000) J. Biol. Chem., 275:13343-13348
30. Smith and Waterman (1981) Advances in Applied Mathematics, 2:482 489
31. Strack et al. (1998) J Comp Neurol. 1998 392:515-527.
32. Towbin et al. (1979) Proc Nat Acad Sci USA, 76:4350-4354
33. Usui et al. (1988) J Biol Chem. 263:3752-3761
34. Wen et al. (2003) World J Gastroenterol. 9:1342-1346
35. Wu et al. (1989) Proc. Natl. Acad. Sci. USA. 86:2757-2760

1-12. (canceled)

13. A method of assessing the efficiency of a modulator of a PP2A phosphatase comprising a PP2A/Bγ subunit for the treatment of a mental disorder, said method comprising administering said modulator to an animal model for said mental disorder; wherein a determination that said modulator ameliorates a representative characteristic of said mental disorder in said animal model indicates that said agonist is a drug for the treatment of said mental disorder.

14. The method of claim 13, wherein said animal model is the STOP−/− mice with synaptic defects and severe behavioral disorders.

15. The method of claim 13, wherein said modulator specifically modulates a PP2A phosphatase comprising the PP2A/Bγ subunit.

16. The method of claim 13, wherein said mental disorder is selected from the group consisting of bipolar disorder, schizophrenia and depression.

17. The method of claim 16, wherein said mental disorder is bipolar disorder.

18-42. (canceled)

43. A method of screening candidate modulator compounds of a Protein Phosphatase 2A (PP2A) phosphatase comprising the steps of:
a) contacting PP2A/Bγ or a PP2A phosphatase comprising a PP2A/Bγ subunit with the candidate modulator compound; and
b) testing the activity of PP2A/Bγ or of the PP2A phosphatase comprising a PP2A/Bγ subunit in the presence of said candidate compound,
wherein a difference in the activity of PP2A/Bγ or of the PP2A phosphatase comprising a PP2A/Bγ subunit in the presence of said compound in comparison to the activity in the absence of said compound indicates that the compound is a modulator of PP2A/Bγ or of the PP2A phosphatase comprising a PP2A/Bγ subunit.

44. The method according to claim 43, wherein a PP2A/Bγ subunit is contacted with the candidate modulator compound.

45. The method according to claim 43, wherein said candidate modulator compound is selected from the group consisting of a natural ligand, a small molecule, an antibody, an antisense RNA, an aptamer and a short interfering RNA.

46. A method of treating a mental disorder comprising the administration of a modulator of a PP2A phosphatase to an individual in an mount effective to treat said mental disorder.

47. The method according to claim 46, further comprising the administration of a known drug for said treatment of said mental disorder.

48. The method according to claim 46, wherein said modulator is a gene therapy vector comprising a polynucleotide encoding a PP2A/Bγ subunit.

49. The method according to claim 46, wherein said mental disorder is selected from the group consisting of bipolar disorder, schizophrenia and depression.

50. A method comprising determining the identity of a nucleotide at a PP2A/Bγ-related biallelic marker or the complement thereof in a biological sample.

51. The method according to claim 50, wherein said biological sample is derived from a single individual.

52. The method according to claim 51, wherein the identity of the nucleotides at said biallelic marker is determined for both copies of said biallelic marker present in said individual's genome.

53. The method according to claim 52, wherein said determining is performed by a microsequencing assay.

54. The method according to claim 52, further comprising amplifying a portion of said sequence comprising the biallelic marker prior to said determining step.

55. The method according to claim 54, wherein said amplifying is performed by PCR.

56. The method according to claim 50, wherein said genotyping step identifies a PP2A/Bγ-related biallelic marker selected from the group consisting of 99-24169/139, 24-257/320, 99-24175/218 and 24-247/216 (as depicted in table 3A) and the complements thereof.

57. The method according to claim 56, further comprising the step of correlating the result of the genotyping step with a risk of suffering from a mental disorder.

58. The method according to claim 57, wherein presence of a genotype “AA” at biallelic marker 99-24169/139 is indicative of a risk of suffering from a mental disorder.

59. The method according to claim 57, wherein the presence a haplotype “AG” at biallelic markers 24169/139 and 24-247/216 is indicative of a risk of suffering from a mental disorder.

60. The method according to claim 57, wherein presence of a haplotype “AA” at biallelic marker 24-257/320 and 99-24175/218 is indicative of a risk of suffering from a mental disorder.

61. The method according to claim 57, wherein said mental disorder is selected from the group consisting of bipolar disorder, schizophrenia and depression.

62. The method according to claim 61, wherein said mental disorder is bipolar disorder.
